(19) United States
(12) Patent Application Publication
(10) Pub. No.: US 2010/0322951 A1
(43) Pub. Date: Dec. 23, 2010

(54) Replication-proficient dsRNA capsids and uses thereof

(75) Inventors: David Hone, Poolesville, MD (US); David Onyabe, Poolesville, MD (US)

Correspondence Address: MDIP LLC, POST OFFICE BOX 2630, MONTGOMERY VILLAGE, MD, 20886-2630, US

(73) Assignee: BACILLIGEN, INC., Rockville, MD (US)

(21) Appl. No.: 12/521,768

(22) PCT Filed: Dec. 31, 2007
(86) PCT No.: PCT/US07/89209

§ 371 Date: Aug. 27, 2010

Related U.S. Application Data
(60) Provisional application No. 60/882,679, filed on Dec. 29, 2006.
(60) Provisional application No. 60/972,097, filed on Sep. 13, 2007.

(51) Int. Cl. A61K 39/00; A61P 35/00; A61P 33/06; A61P 31/18; A61P 31/06; C12N 15/63; C12N 5/00; C12N 1/00

(52) U.S. Cl. 424/184.1; 435/320.1; 435/325; 435/410; 435/243

(57) ABSTRACT

Compositions and methods useful in the production of proteins in vitro and in vivo are provided. In particular, this invention provides bionanoparticles comprised of replication-proficient, double-stranded RNA (dsRNA) capsids which are capable of transfecting, for example, target bacterial, yeast, plant and mammalian cells, and causing expression of genes of interest in said transfected cells. The bionanoparticles comprised of replication-proficient, dsRNA capsids are also capable of expressing genes of interest in vitro. The genes of interest can encode proteins that are useful, for example, as research reagents, therapeutics and vaccines. Also described are methods that enable the construction of bionanoparticles comprised of replication-proficient dsRNA capsids and methods for transfecting cells with said bionanoparticles and expressing said genes of interest in transfected cells and in vitro.

Portions of the research described herein were developed using funds from the U.S. government under Grant No. RO1-AI-055367.

### BACKGROUND OF INVENTION

The instant application contains a Sequence Listing which has been submitted in ASCII format via EFS-Web and is hereby incorporated by reference in its entirety. Said ASCII copy, created on Aug. 25, 2010, is named 20814339.txt and is 36,470 bytes in size.

The elevated threat of bioterrorism and the renewed threat of emerging pathogens, such as H5N1 influenza A virus, have caused a change in government and public health priorities, which now call for the development of an array of defense measures against this insidious form of weaponry and to provide real-time protection against emerging pathogens. Although numerous toxins and microbial pathogens are considered high risk candidates, it is possible to develop therapeutic and preventive pharmaceutical stockpiles as countermeasures to known biological threat agents. The shortfall of that approach is the high cost to tax payers of stockpile maintenance, as the therapeutic and preventive countermeasures have finite shelf half-lives, and genetic modifications can be made to render known bioweapons insensitive to conventional countermeasures. Moreover, the emergence of new serotypes of influenza and other viruses that cause high human mortality has revealed another gap in preparing for biological threats.

Those factors have engendered a need to develop novel strategies that provide a blanket defense against existing and emerging biological threats that can be deployed rapidly as a countermeasure in the event of unnatural or natural outbreaks due to bioweapons or emergent pathogens. One such strategy is to develop a highly flexible platform technology that enables the rapid production of biochemicals, bioactive RNA, carbohydrates, lipids, and proteins, such as, scavenger proteins, receptor antagonists, vaccine antigens or monoclonal antibodies, that serve as therapeutic or prophylactic countermeasures against looming and actual biological threats.

Given the fact that many therapeutic and prophylactic countermeasures, such as proteins, possess complex 3-dimensional structures for therapeutic or prophylactic activity, usually, only the cognate biological expression system is capable of producing such molecules in native conformations and with optimal bioactivity. Such include, for example, bacterial, yeast and plant expression systems. However, there are a number of therapeutic and prophylactic countermeasures that require precise mammalian glycosylation patterns and other post translational modifications to form an appropriate conformation.

However, the generation of stable mammalian cell lines is time consuming and laden with risk, such that short-term, high-yield expression in mammalian cells is considered more amenable to the rapid production of therapeutic and prophylactic countermeasures against bioweapons and emergent pathogens. Given the array of expression systems that are needed to provide umbrella readiness for the production of any therapeutic or prophylactic countermeasure, vector systems that are highly flexible and capable of shuttling and expressing cargo genes in multiple lineages offer significant cost, logistical, regulatory and time-saving advantages. However, the prior art does not provide compositions or methods with said capabilities.

Double-stranded RNA phage (dsRP) resemble members of the reoviridae family (1-6). The distinguishing attributes of dsRP are a genome comprised of three double-stranded RNA (dsRNA) segments (2-4, 7) and a lipid-containing membrane coat (6, 8-12). The genomic segments are contained within the capsid core, which is comprised of proteins P1, P2, P4, and P7, and are produced by genes encoded on the 7051 by dsRNA segment, designated “segment-L” (GenBank Accession No. AF226851). Synthesis of positive-strand RNA (i.e. mRNA) occurs within the capsid, which is carried out by RNA-dependent RNA polymerase encoded by gene-2 on segment-L (4, 13). P1 provides helicase activity and P4 forms a hexamer and acts as a portal for RNA entry and secretion to and from the capsid. P7 plays a pivotal role in capsid stability (14, 15). The capsid is completed by the addition of P8, which occurs after completion of dsRNA synthesis, forming an outer proteinaceous sheath (16, 17). P8 plays a pivotal role in capsid translocation across membranes (16, 17).

DsRP phi-6 was the first member of this family to be described in detail, and normally phi-6 infects and replicates in Pseudomonas syringae (5). Other dsRP, such as phi-8, phi-11, phi-12 and phi-13, also replicate in Pseudomonas syringae as the natural host but are capable of forming transient carrier states in Escherichia coli and Salmonella typhimurium (5, 18-20). Inserting a kanamycin-resistance allele into segment-M of dsRP φ6 enabled maintenance of carrier strains over several passages (21). In one study, the wild-type translucent plaque phenotype of dsRP in carrier strains was maintained for up to five passages. After additional passages, however, newly formed dsRP lost the ability to form plaques (21). Carrier strains typically lost the ability to produce infectious phage all together, yet capsids and dsRNA segments were continuously maintained in the cytosol of the carrier strain. The dsRP from such bacterial strains displayed deletions in one of more of the dsRNA segments (21) and it was possible through this procedure to isolate a mutant dsRP that lacked segment-S from one carrier strain that had lost the capacity to produce infectious phage but continued the capacity to generate kanamycin-resistance nucleocapsids (21, 22).

U.S. Pat. No. 7,018,835 asserts that recombinant dsRP (rdsRP) are useful for the expression of vaccine antigens, bioactive proteins, immunoregulatory proteins, antisense RNAs, and catalytic RNAs in mammalian cells or tissues. U.S. Pat. No. 7,018,835 proposes that batches of rdsRP can be generated by replicating a parent dsRP in the bacterial transformants that carry plasmids expressing a recombinant segment. However, U.S. Pat. No. 7,018,835 does not provide rdsRP which are independent of the wild-type helper phage for propagation and does not provide rdsRP that are separated from the wild type dsRP (U.S. Pat. No. 7,018,835).

Recently, US Publ. No. 20060115493 (2006) suggested that it is possible to engineer recombinant dsRNA nucleocapsids (rdsRNs) that are capable of expressing multiple antigens in mammalian cells, and that such rdsRNs are useful as vaccine vectors. A mammalian RNA sequence encoding an internal ribosome entry site (TRES) 5′ to a gene of interest, to enable expression of vaccine antigens in mammalian cell was used. The expression cassettes were inserted into segment-M and segment-S of dsRNA phage. To propagate the rdsRN, US Publ. No. 20060115493 (2006) teaches a balanced-lethal maintenance system in which a deletion in the genomic selectable trait (e.g. deleted asd gene) of the carrier strain was complemented by an expression cassette encoding the trait (e.g. asd gene) in the rdsRN. The system was launched by introducing the mRNA of recombinant segment S and segment M into a target bacterial strain by electroporation. The target strain expressed segment-L on a plasmid and thereby produced procapsids. The recombinant RNA along with the segment-L RNA were incorporated into the procapsids to form mature rdsRNs that were capable of sustained replication within the bacterial carrier strain.

However, US Publ. No. 20060115493 (2006) is largely restricted to the use of rdsRNs in bacterial vectors and is devoted entirely to expression of rdsRN-encoded cargo genes in mammalian cells following introduction via a bacterial delivery vehicle.

US Publ. No. 20060115493 (2006) suggests that rdsRNs can be purified from carrier strains using methods described in Mindich et al., J Virol 66, 2605-10 (1992), Mindich et al., Virology 212:213-217 (1995) Mindich et al., J Bacteriol 181:4505-4508 (1999), Qiao et al., Virology 275:218-224 (2000), Qiao et al., Virology 227:103-110 (1997), Olkkonen et al., Proc Natl Acad Sci USA 87:9173-9177 (1990) and Onodera et al., J Virol 66, 190-196 (1992). However, as shall be revealed below, those methods do not produce useful batches of rdsRN and are not enabling.

The developments (U.S. Pat. No. 7,018,835; and US Publ. No. 20060115493) bode well for emerging bionanotechnology; yet, there remains a need to develop a recombinant dsRNA capsid system and methods that efficiently produce, enable purified preparations and are capable of shuttling and expressing cargo therapeutic or prophylactic countermeasures in multiple lineages, including monera, plantae and mammalian hosts, in vitro and in vivo.

### SUMMARY OF THE INVENTION

An object of the present invention is to provide replication-proficient dsRNA capsids (RPCs) that produce high yields of capsids in carrier strains.

A further object of the present invention is to provide RPCs that are optimized for expression of messenger RNA (herein referred to as “mRNA”) in mammalian cells.

Another object of the present invention is to provide RPCs that are optimized for expression of proteins in mammalian cells.

Still another object of the present invention is to provide RPCs that are optimized for expression of secreted proteins in mammalian cells.

A further object of the present invention is to provide RPCs that are optimized for expression of mRNA in plant cells.

Another object of the present invention is to provide RPCs that are optimized for expression of proteins in plant cells.

Still another object of the present invention is to provide RPCs that are optimized for expression of secreted proteins in plant cells.

A further object of the present invention is to provide RPCs that are optimized for expression of mRNA in yeast cells.

Another object of the present invention is to provide RPCs that are optimized for expression of proteins in yeast cells.

Still another object of the present invention is to provide RPCs that are optimized for expression of secreted proteins in yeast cells.

A further object of the present invention is to provide RPCs that are optimized for expression of mRNA in bacterial cells.

Another object of the present invention is to provide RPCs that are optimized for expression of proteins in bacterial cells.

Still another object of the present invention is to provide RPCs that are optimized for expression of secreted proteins in bacterial cells.

### BRIEF DESCRIPTION OF THE DRAWINGS

FIG. 1 depicts representative changes to the termini of a message to enhance replication. Wild type and modified termini of phage φ6 L, M and S segments are presented. The wild type L segment +ve strand 5′ terminus sequence is SEQ ID NO:50 and the 3′ terminus sequence is SEQ ID NO:3. The L segment −ye strand 5′ terminus sequence is SEQ ID NO:51 and the 3′ terminus sequence is SEQ ID NO:52. The wild type M segment +ve strand 5′ terminus sequence is SEQ ID NO:54 and the 3′ terminus sequence is SEQ ID NO:3. The M segment −ye strand 5′ terminus sequence is SEQ ID NO:51 and the 3′ terminus sequence is SEQ ID NO:53. The wild type S segment +ve strand 5′ terminus sequence is SEQ ID NO:54 and the 3′ terminus sequence is SEQ ID NO:3. The S segment −ye strand 5′ terminus sequence is SEQ ID NO:51 and the 3′ terminus sequence is SEQ ID NO:53. The replication proficient L segment +ve strand 5′ terminus sequence is SEQ ID NO:54 and the 3′ terminus sequence is SEQ ID NO:4. The L segment −ye strand 5′ terminus sequence is SEQ ID NO:55 and the 3′ terminus sequence is SEQ ID NO:80. The replication proficient M segment +ve strand 5′ terminus sequence is SEQ ID NO:54 and the 3′ terminus sequence is SEQ ID NO:4. The M segment −ye strand 5′ terminus sequence is SEQ ID NO:55 and the 3′ terminus sequence is SEQ ID NO:53. The replication proficient S segment +ve strand 5′ terminus sequence is SEQ ID NO:54 and the 3′ terminus sequence is SEQ ID NO:4. The S segment −ye strand 5′ terminus sequence is SEQ ID NO:55 and the 3′ terminus sequence is SEQ ID NO:53.

FIG. 2 depicts representative changes to the termini of a message to enhance replication. Wild type and modified termini of phage φ8 L, M and S segments are presented. The wild type L segment +ve strand 5′ terminus sequence is SEQ ID NO:56 and the 3′ terminus sequence is SEQ ID NO:57. The L segment −ye strand 5′ terminus sequence is SEQ ID NO:58 and the 3′ terminus sequence is SEQ ID NO:60. The wild type M segment +ve strand 5′ terminus sequence is SEQ ID NO:61 and the 3′ terminus sequence is SEQ ID NO:63. The M segment −ye strand 5′ terminus sequence is SEQ ID NO:59 and the 3′ terminus sequence is SEQ ID NO:65. The wild type S segment +ve strand 5′ terminus sequence is SEQ ID NO:61 and the 3′ terminus sequence is SEQ ID NO:64. The S segment −ye strand 5′ terminus sequence is SEQ ID NO:81 and the 3′ terminus sequence is SEQ ID NO:65. The replication proficient L segment +ve strand 5′ terminus sequence is SEQ ID NO:62 and the 3′ terminus sequence is SEQ ID NO:82. The L segment −ye strand 5′ terminus sequence is SEQ ID NO:61 and the 3′ terminus sequence is SEQ ID NO:66. The replication proficient M segment +ve strand 5′ terminus sequence is SEQ ID NO:61 and the 3′ terminus sequence is SEQ ID NO:82. The M segment −ye strand 5′ terminus sequence is SEQ ID NO:61 and the 3′ terminus sequence is SEQ ID NO:65. The replication proficient S segment +ve strand 5′ terminus sequence is SEQ ID NO:61 and the 3′ terminus sequence is SEQ ID NO:82. The S segment −ye strand 5′ terminus sequence is SEQ ID NO:61 and the 3′ terminus sequence is SEQ ID NO:65.

FIG. 3 depicts representative changes to the termini of a message to enhance replication. Wild type and modified termini of phage φ13 L, M and S segments are presented. The wild type L segment +ve strand 5′ terminus sequence is SEQ ID NO:67 and the 3′ terminus sequence is SEQ ID NO:68. The L segment −ye strand 5′ terminus sequence is SEQ ID NO:69 and the 3′ terminus sequence is SEQ ID NO:70. The wild type M segment +ve strand 5′ terminus sequence is SEQ ID NO:72 and the 3′ terminus sequence is SEQ ID NO:73. The M segment −ye strand 5′ terminus sequence is SEQ ID NO:74 and the 3′ terminus sequence is SEQ ID NO:71. The wild type S segment +ve strand 5′ terminus sequence is SEQ ID NO:72 and the 3′ terminus sequence is SEQ ID NO:75. The S segment −ye strand 5′ terminus sequence is SEQ ID NO:76 and the 3′ terminus sequence is SEQ ID NO:71. The replication proficient L segment +ve strand 5′ terminus sequence is SEQ ID NO:54 and the 3′ terminus sequence is SEQ ID NO:77. The L segment −ye strand 5′ terminus sequence is SEQ ID NO:78 and the 3′ terminus sequence is SEQ ID NO:82. The replication proficient M segment +ve strand 5′ terminus sequence is SEQ ID NO:83 and the 3′ terminus sequence is SEQ ID NO:77. The M segment −ye strand 5′ terminus sequence is SEQ ID NO:78 and the 3′ terminus sequence is SEQ ID NO:84. The replication proficient S segment +ve strand 5′ terminus sequence is SEQ ID NO:83 and the 3′ terminus sequence is SEQ ID NO:77. The S segment −ye strand 5′ terminus sequence is SEQ ID NO:78 and the 3′ terminus sequence is SEQ ID NO:84.

FIG. 4 depicts a schematic construct. pac is a sequence for packaging RNA in the capsid. UTR is an untranslated region. Kozac is a Kozak sequence, generally recognized by a ribosome as a translation start region. PTB is pyrimidine tract binding. pol is a polymerase binding site.

### DETAILED DESCRIPTION OF THE INVENTION

These embodiments have been enabled in the present invention by providing novel compositions comprised of replication-proficient capsids (herein referred to as “RPCs”) that are useful in bacterial delivery vehicles and independently as purified capsids capable of expressing cargo genes or genetic-trait modifying RNA sequences in vitro and in vivo in, for example, bacterial, yeast, plant and mammalian cells. For the purpose of the instant invention, a cargo gene is considered equivalent to a transgene, target gene, expressed gene, foreign gene, expressed sequence, foreign sequence, heterologous gene and so on, terms known and used in the art to describe that nucleic acid engineered to be carried by a vector, which nucleic acid encodes a product of interest, such as a polypeptide or an RNA. As the instant invention relates to double stranded RNA viruses, the gene, transgene, expressed sequence and so on is an RNA.

Critical factors that were not considered in the above-cited developmental studies on rdsRPs (U.S. Pat. No. 7,018,835), which may carry segment-S and/or segment-M, both of which carry genes associated with lysis, and rdsRNs (US Publ. No. 20060115493) that confine those prototype technologies to the realm of bacterial nucleic acid delivery vehicles (e.g. Hone et al., Microb Path. 6:407 (1988)) and will prevent the use of rdsRPs and rdsRNs independently of bacterial vectors for expression in vitro and in vivo include:

(i) the yield of rdsRP/rdsRN, such as, in a mixture, in bacterial production strains;

(ii) the lack of compositions and methods for the purification of capsids;

(iii) factors that enable translation of rdsRP/rdsRN-derived mRNA in host cells;

(iv) compositions that efficiently replicate bacterial hosts and methods to purify and use rdsRPs/rdsRNs independently of bacterial delivery vehicles; and

(v) compositions and methods to enable translation of rdsRP/rdsRN-derived mRNA in bacterial, yeast and plant cells, which is a prerequisite that will allow those systems to be developed as a flexible expression platform for rapid response scenarios described above.

With regard to translation of rdsRP/rdsRN-derived mRNA in host cells, the prior art (i.e. U.S. Pat. No. 7,018,835; and US Publ. No. 20060115493) teach the use of a cap-independent translation enhancer (herein referred to as “CITE”) and internal ribosome entry site (herein referred to as “IRES”) sequences to initiate expression of a gene of interest in mammalian cells. However, as will be shown below, the described compositions in U.S. Pat. No. 7,018,835 and US Publ. No. 20060115493 carry sequences that are insufficient to enable measurable expression of recombinant proteins in mammalian cells. It is contended herein, therefore, that U.S. Pat. No. 7,018,835 and US Publ. No. 20060115493 do not enable the translation of rdsRP/rdsRN-encoded genes in target mammalian host cells.

To remedy these deficiencies, the present invention, on the other hand, provides novel compositions comprised of replication-proficient capsids (herein “RPCs”), which are derived from dsRP useful for expressing genes and trait modifying RNA in, for example, bacterial, yeast, plant and mammalian cells in vitro and in vivo. The present invention, also provides novel compositions, and methods thereof, which are useful for expression of carbohydrates, lipids, proteins, glycoproteins, gene silencing, genetic trait modification and gene therapeutics, either alone or in combination, such as with a cytotoxic agent or combinations thereof.

Replication proficiency is observed in the practice of the instant invention by the observation that the prior art materials did not demonstrate adequate replication in bacteria to provide practical numbers of capsids. Many of the prior art preparations contained a substantial preponderance of procapsids, which, as known in the art, are the protein shells of a virus lacking the nucleic acids within, and thus are expression incompetent. A capsid capable of replication is a procapsid containing the full complement of dsRNA and a p8 coat. For the purposes of the instant invention, replication proficiency relates to a configuration that yields a preparation resulting in at least 5 ng of capsids per 10⁹ bacteria, at least 6 ng, at least 7 ng, at least 8 ng, at least 9 ng, at least 10 ng, at least 11 ng, at least 12 ng, at least 13 ng, at least 14 ng, at least 15 ng, at least 16 ng, at least 17 ng, at least 18 ng, at least 19 ng, at least 20 ng or more capsids per 10⁹ bacteria. An alternative metric to assess replication proficiency is the ratio of capsids to procapsids in a preparation. Thus, replication proficiency relates to a configuration that yields a preparation containing at least 1 capsid to 20 procapsids (1:20), at least 1:19, at least 1:18, at least 1:17, at least 1:16, at least 1:15, at least 1:14, at least 1:13, at least 1:12, at least 1:11, at least 1:10, at least 1:9, at least 1:8, at least 1:7, at least 1:6, at least 1:5, at least 1:4, at least 1:3, at least 1:2, at least 1:1, at least 1:0.9, at least 1:0.8, at least 1:0.7, at least 1:0.6, at least 1:0.5, at least 1:0.4, at least 1:0.3, at least 1:0.2, at least 1:0.1 or more capsids to procapsids in a preparation.

Viral replication is a complex process that may or may not impact the host transcription and translation mechanisms, will require the ability to overcome any host defense mechanisms, will have to ensure the adequate and properly timed production of viral proteins and viral nucleic acids to ensure the orchestrated assembly of the complex virus structure and so on to ensure the development of a capsid. The first two factors relating to the bacterial host impact on virus replication are not understood. In certain circumstances, there are involved timed interactions between the host transcription and translation machineries and the virus transcription and translation machineries to produce a capsid. Those uncertainties impact the need for high titer preparations of recombinant virus, see for example, the current lack of reproducible methods for making recombinant lentivirus vectors and recombinant AAV vectors.

Notwithstanding that environment, the instant invention relates to the RPC technology that enables high titer production of dsRNA capsids and the expression of a transgene at levels at least two times, at least three times, at least four times, at least five time, at least six times, at least seven time, at least eight times, at least nine times, at least ten times or more greater than that observed using rdsRP or rdsRN technology, for example, in an in vitro translation system. Those goals were achieved, in part, by manipulating not one but both termini of the message, and the unexpectedness of having those multiply manipulated nucleic acids operate in a proper fashion in a host cell to produce a capsid.

### Recombinant DNA Materials and Methods

The recombinant DNA procedures used in the construction of the strains, bacterial strains and RPCs, include but are not limited to, polymerase chain reaction (PCR), restriction endonuclease (RE) digestions, DNA ligation, agarose gel electrophoresis, DNA purification, and dideoxynucleotide sequencing, are known in the art (Miller, A Short Course in Bacterial Genetics, Cold Spring Harbor Laboratory Press, Cold Spring Harbor, N.Y.; 1992); (Bothwell et al., eds., Methods for Cloning and Analysis of Eukaryotic Genes, Jones & Bartlett Publishers Inc., Boston, Mass. (1990); and (Ausubel et al., eds., Current Protocols in Molecular Biology, Greene Publishing Associates and Wiley-Intersciences, John Wiley and Sons, New York, N.Y. (2007), bacteriophage-mediated transduction (de Boer et al., Cell, 56:641-649 (1989); (Miller, supra, 1992) and (Ausubel et al., eds., The polymerase chain reaction. John Wiley & Sons, New York, N.Y (1990), or chemical (Bothwell et al.; supra; Ausubel et al., eds., Short Protocols in Molecular Biology, John Wiley and Sons, New York, N.Y. (1992); Feigner et al., Proc. Natl. Acad. Sci. USA 84:7413-7417 (1987); and Farhood, Annal. N.Y. Acad. Sci. 716:23-34 (1994), electroporation (Bothwell et al.; supra; Ausubel et al., 2007 supra; and Sambrook et al., Molecular Cloning: A Laboratory Manual, Cold Spring Harbor Laboratory Press, Cold Spring Harbor, N.Y.; 1992) and physical transformation techniques (Johnston et al., Meth. Cell Biol, 43(Pt A):353 365 (1994); Bothwell et al.; supra). The genes can be incorporated on phage (de Boer et al., Cell, 56:641-649; 1989), plasmids vectors (Curtiss et al., Infect. Immun., 55:3035-3043 (1987) or spliced into the chromosome (Hone et al., Microbial. Path. 5:407-418 (1988) of the target strain.

REs (New England Biolabs, Beverly, Mass.), T4 DNA ligase (New England Biolabs, Beverly, Mass.) and Taq polymerase (Life Technologies, Gaithersburg, Md.) were used according to the manufacturers' protocols. Plasmid DNA was prepared using small-scale (Qiagen Miniprep™ kit, Santa Clarita, Calif.) or large-scale (Qiagen Midiprep™ kit, Santa Clarita, Calif.) plasmid DNA purification kits according to the manufacturer's protocols (Qiagen, Santa Clarita, Calif.). Nuclease-free, molecular biology grade deionized water, Tris-HCl (pH 7.5), EDTA pH 8.0, 1M MgCl₂, 100% (v/v) ethanol, ultra-pure agarose, and agarose gel electrophoresis buffer were purchased from Life Technologies, Gaithersburg, Md. RE digestions, PCRs, DNA ligation reactions and agarose gel electrophoresis were conducted according to well-known procedures (Sambrook et al., supra; and Ausubel et al., 2007, supra). Nucleotide sequencing to verify the DNA sequence of each recombinant plasmid described in the following examples was accomplished by conventional automated DNA sequencing techniques using an Applied Biosystems automated sequencer, model 373A.

PCR primers were purchased from Integrated DNA Technologies (Coralville, Iowa) or the University of Maryland Biopolymer Facility (Baltimore, Md.) and were synthesized using an Applied Biosystems DNA synthesizer (model 373A). PCR primers were used at a concentration of 50-500 μM, preferably 200 μM, and annealing temperatures for the PCR reactions were determined using Clone manager software version 4.1 (Scientific and Educational Software Inc., Durham, N.C.) or OLIGO primer analysis software version 4.0. The software enable the design of PCR primers and identifies RE sites that are compatible with the specific DNA fragments being manipulated. PCRs were conducted in a Stratagene RoboCycler® 96 Gradient Cycler with Hot Top (Cat No. 400885; LaJolla, Calif.). Primer annealing, elongation and denaturation times in the PCRs were set according to standard procedures (Ausubel et al; supra). The products of RE digestions and PCRs were analyzed by agarose gel electrophoresis using standard procedures (Ausubel et al.; supra); and Sambrook et al.; supra). A positive clone was defined as one that displays the appropriate RE pattern and/or PCR pattern. Plasmids identified through this procedure are further evaluated using dideoxy DNA sequencing procedures, as known in the art.

Escherichia coli strains Top10 and DH5α are purchased from Invitrogen (Carlsbad, Calif.) and strain SCS110 is purchased from Stratagene (La Jolla, Calif.) Recombinant plasmids are introduced into E. coli by electroporation using a Gene Pulser (BioRad Laboratories, Hercules, Calif.), for example, set at 200Ω, 25 uF and 1.8 kV, or by chemical transformation, as described previously (Ausubel et al., 2007 supra).

Bacterial strains were grown on tryptic soy agar (Difco, Detroit, Mich.) or in tryptic soy broth (Difco, Detroit, Mich.), unless otherwise stated, at an appropriate temperature (e.g. 25° C. and 37° C.). Media were supplemented with ampicillin (for example, 100 mg/ml), kanamycin (for example, 50 mg/ml), and/or chloramphenicol (for example, 20 mg g/ml) (Sigma, St. Louis, Mo.) as needed. Bacterial strains were stored in an Ultra-Low Temperature VIP® Freezer (Sanyo Electric Biomedical Co., Ltd., Osaka, Japan, Cat No. MDF-U32V) at −80° C. in tryptic soy broth (Difco) containing 30% (v/v) glycerol (Sigma, St Louis, Mo.) at ca. 10⁹ colony-forming units (herein referred to as “cfu”) per ml.

### Reagent List

KpnI (New England Biolabs, Beverly, Mass., Cat. Nos. R0142S), PstI (New England Biolabs, Beverly, Mass., Cat. No. R0140S), Tryptic Soy broth (Difco, Detroit, Mich., Cat. No. 211822), Tryptic Soy agar (Difco, Detroit, Mich., Cat. No. 236920), Miniprep® plasmid DNA purification kit (Qiagen, Valencia, Calif., Cat. No. 27106), glycerol (Sigma, St. Louis, Mo., Cat. No. G5516), HpaI (New England Biolabs, Beverly, Mass., Cat. No. R0105S), calf intestinal alkaline phosphatase (New England Biolabs, Beverly, Mass., Cat. No. M0290S), Vent® DNA polymerase (New England Biolabs, Cat. No. M0254S), QIAquick PCR purification kit (Qiagen, Cat. No. 28106, Valencia, Calif.), diaminopimelic acid (Sigma-Aldrich, St. Louis, Mo., Cat. No. D1377), BglII (New England Biolabs, Beverly, Mass., Cat No. R0144S), IPTG (Invitrogen, Carlsbad, Calif., Cat. No. 15529-019), cell culture lysis reagent (Promega, Madison, Wis., Cat. No. E1531), lysozyme (Sigma, St. Louis, Mo., Cat. No. L6876), potassium phosphate (Sigma, St. Louis, Mo., Cat. No. P5379), magnesium chloride (Sigma, St. Louis, Mo., Cat. No. M1028) DraIII (New England Biolabs, Beverly, Mass., Cat. No. R0510S), PsiI (New England Biolabs, Beverly, Mass., Cat. No. V0279S), proteinase K (Ambion, Austin, Tex., Cat. No. 2542-2548), Durascribe® T7 transcription kit (Epicentre, Madison, Wis.), Durascribe® SP6 transcription kit (Epicentre, Madison, Wis.), MEGAscript® MEGAscript® T7 transcription kit (Ambion, Austin, Tex., Cat. No. 1334), MEGAscript® SP6 transcription kit (Ambion, Austin, Tex., Cat No. 1330), MEGAclear® columns (Ambion, Austin, Tex., Cat No. 1908), BrightStar® biotinylated RNA millennium marker (Ambion, Austin, Tex., Cat. No. 7170), BrightStar® nylon membrane (Ambion, Austin, Tex., Cat. No. 10102), BrightStar® Biodetect kit (Ambion, Austin, Tex., Cat. No. 1930), Tris-HCl buffer (Quality Biological, Gaithersburg, Md., Cat. No. 351-007-100), magnesium chloride (Sigma-Aldrich, St. Louis, Mo., Cat. No. M1787), ammonium acetate (Sigma-Aldrich, St. Louis, Mo., Cat. No. A2706), sodium chloride (Sigma-Aldrich, St. Louis, Mo., Cat. No. S7653), potassium chloride (Sigma-Aldrich, St. Louis, Mo., Cat. No. P3911), dithiothreitol (Sigma-Aldrich, St. Louis, Mo., Cat. No. D9779), EDTA (Sigma-Aldrich, St. Louis, Mo., Cat. No. E8008), polyethylene glycol 4000 (Fluka, Buchs, Switzerland, Cat. No. 95904), SUPERase® RNase inhibitor (Ambion, Austin, Tex., cat. No 2694,), biotin-14-CTP (Invitrogen, Carlsbad, Calif., Cat. No. 19519-016), and RNase one ribonuclease (Promega, Madison, Wis., Cat. No. M4261) were used.

The RPCs of the present invention are comprised of at least one recombinant dsRNA (herein rdsRNA) segment. The cDNA sequences encoding the rdsRNA segments can be generated using convention recombinant DNA techniques involving DNA synthesis (e.g. using a commercial vendor such as DNA 2.0 (Menlo Park, Calif.)), PCR, RE digestion and assembly of the elements using T4 ligase. Alternatively, recombinant segments can be fully generated synthetically using an Applied Biosystems ABI™ 3900 High-Throughput DNA Synthesizer (Foster City, Calif. 94404 U.S.A.) and procedures provided by the manufacturer. To synthesize the cDNA copies of segment-L and the recombinant segments, a series of segments of the full-length sequence are generated by PCR and are ligated together to form the full-length segment using procedures well know in the art (Haas et al., Curr Biol 6:315; 1996; Andre et al., J Virol 72:1497; 1998; Fouts et al., J Virol 74:11427; 2000). Briefly, synthetic oligonucleotides 100-200 nucleotides in length (i.e. preferably with sequences at the 5′-end and the 3′-end that match at the 5′-end and the 3′-end of the oligonucleotides that encode the adjacent sequence) are produced using an automated DNA synthesizer (e.g. Applied Biosystems ABI™ 3900 High-Throughput DNA Synthesizer (Foster City, Calif.)). Using the same approach, the complement oligonucleotides are synthesized and are annealed with the complementary partners to form double stranded oligonucleotides. Pairs of double stranded oligonucleotides (i.e. those that encode adjacent sequences) and joined by ligation to form a larger fragment. The larger fragments are purified by agarose gel electrophoresis and isolated using a gel purification kit (e.g. The QIAEX® II Gel Extraction System, from Qiagen, Santa Cruz, Calif., Cat. No. 12385). That procedure is repeated until the full-length DNA molecule is created. After each round of ligation, the fragments can be amplified by PCR to increase the yield. Procedures for de novo synthetic gene construction are well known in the art and are described elsewhere (Andre et al. supra, (1998); Haas; supra, (1996)); alternatively synthetic genes can be purchased commercially, e.g. from the Midland Certified Reagent Co. (Midland, Tex.) or from DNA 2.0 (Menlo Park, Calif.).

Mutagenesis of dsRNA Segments

Modifications to dsRNA segments can be introduced by employing non-specific mutagenesis either chemically, using agents such as N-methyl-N′-nitro-N-nitrosoguanidine; or using specific recombinant DNA techniques, such as, with PCR-directed mutagenesis or in vitro site-directed mutagenesis (Hutchinson et al., J. Biol. Chem. 253:6551; (1978)), use of the QuikChange® Site-Directed Mutagenesis Kit as directed by the manufacturer (Stratagene, LaJolla, Calif.), the Phusion™ Site-Directed Mutagenesis Kit as directed by the manufacturer (New England Biolabs, Ipswich, Mass.), etc. Additional techniques to introduce mutation-containing nucleic acids into the genomes can be used (see, e.g., Miller 1992, A Short Course in Bacterial Genetics, Cold Spring Harbor Press; Sambrook et al., 1989, Molecular Cloning, A Laboratory Manual, Cold Spring Harbor Laboratory, 2d Ed., Cold Spring Harbor, N.Y.), etc. Recombinant techniques often are preferable since strains constructed by recombinant DNA techniques are far more defined.

### Construction of Replication-Proficient Capsids

It is known in the art that a minor nucleotide change can dramatically alter the copy number of a plasmid per genome (23), resulting in high-copy number plasmids that bear no resemblance in functionality and usefulness to the parent low-copy plasmid from which the mutant was derived. The present invention surprisingly demonstrates that introducing changes to dsRP increases the level of transcription and/or replication resulting in a quantum increase in capsid copy number per bacterial genome, as well as increases the level of capsid-encoded mRNA. The prior art does not provide or appreciate any compositional or methodical guidance on any sort of changes (as used herein) to increase capsid copy number in bacterial hosts. As mentioned above, an embodiment of the present invention has been met, therefore, by providing RPCs which display an increased capsid copy number per host genome in bacterial host strains compared to rdsRP/rdsRN (e.g. U.S. Pat. No. 7,018,835 and US Publ. No. 20060115493) and which express increased levels of capsid-encoded mRNA in host cells.

Overall, there are an array of modifications that enable increase capsid copy number, including, but not limited to increases in RNA synthesis rates, decreases in genome size, increases in replication and/or transcription initiation rates and increases in expression of segment-L and/or P8. Such modifications can be introduced alone or in any combination.

Thus, RPCs of the present invention also can contain at least one recombinant segment that carries at least one modified RNA sequence that results in increased rates of transcription and/or replication rates and thereby increased capsid copy number and/or capsid mRNA levels. The changes required in the RNA segments are not important to the present invention and can be any modification that augments transcription and/or replication rates of at least one genomic segment. Changes can be introduced by standard procedures such as site-directed mutagenesis or insertional mutagenesis as described above. Selection for increased replication rates can be achieved by detecting capsids that impart increased levels of selectable phenotype, such as increased level of antibiotic resistance. For example, capsids with elevated transcription and/or replication rates can increase the tolerance of a host bacterial strain to kanamycin from 100 μg/ml to 500 μg/ml, and as high as 2 mg/ml. Thus, selection for high-copy capsids following mutagenesis can be achieved using concentrations of kanamycin that inhibit the original capsids and allow only hosts bearing the high-copy capsids to grow. Changes in P1, P2 and/or P4 that increase RNA-dependent polymerase and/or helicase activity, which increase RNA synthesis rates above the baseline level of 15-20 nucleotides/min, are preferred. Additional RPC compositions comprise changes in the capsid proteins (for example, P1, P2, P4, P7 and P8) that allow stable replication of reduced genome size, such as those described elsewhere (21), and therefore decrease the overall replication timeframe, are also desirable. Combinations involving mutations that increase the RNA synthesis rate and enable decreased genome size are also useful compositions of the present invention.

In another preferred embodiment, RPCs carry at least one recombinant segment bearing at least one modified RNA terminal end that results in increased rates of transcription and/or replication initiation and thereby increasing capsid copy number and/or capsid mRNA levels. The alterations generally introduce low-melting point stem loops, which are believed to create an artificial priming template that facilitates but are not required for transcription and/or replication initiation (24, 25).

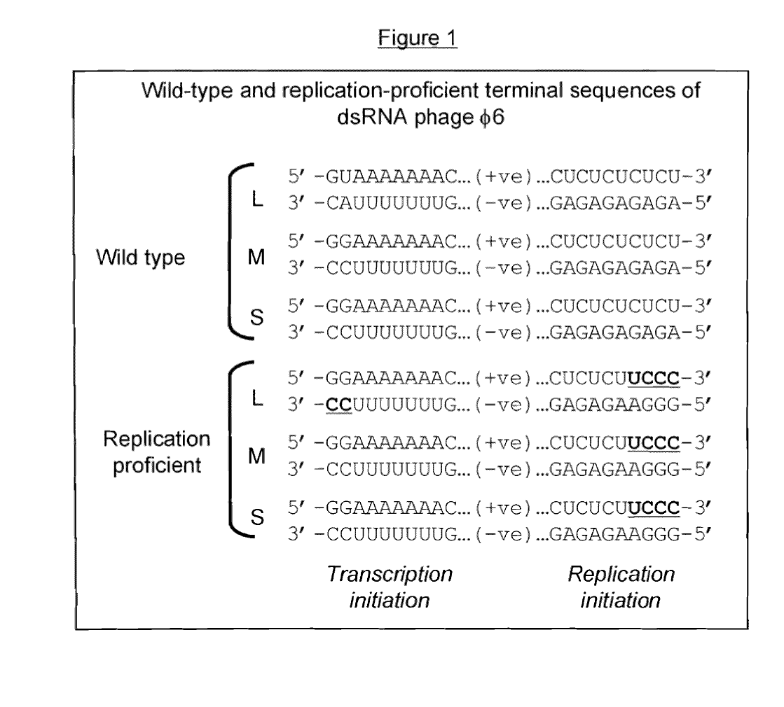

In another embodiment, the altered terminal regions contain added 3′ cytosine residues, which increase transcription and/or replication initiation by P2 (24, 25). By way of an example, which is by no means intended to be limiting, FIG. 1 shows modifying 3′ ends of the negative-strand that increased transcription of phi-6 segment-L up to 5-fold. Hence, an A or G residue, and optionally, a U residue, is replaced by a C using mutagenesis techniques known in the art and described herein. Whether that substitution is effective in increasing capsid production is determined as taught herein, or as known in the art. Expression can be further enhanced if one or more of the endogenous C residues is replaced by a U residue. Generally, the substitutions are at the terminal two, three, four, five, six, seven, eight, nine, ten, eleven, twelve, thirteen, fourteen, fifteen, sixteen, seventeen, eighteen, nineteen or twenty bases. Preferably, the changes are closer to the terminus. The particular modification(s) to a terminus can be tracked by making the modification(s) of interest and determining copy number practicing, for example, quantitative PCR methods known in the art, thus, the instant invention is not limited to a single substitution, but includes plural substitutions as taught herein, such as two substitution, three substitution, four substitutions, five substations, six substitutions and so on.

Transcription of phi-6 segment-L can be increased up to 5-fold by modifying the 3′ end of the negative-RNA (herein referred to as “(−ve)-RNA”) strand from 3′-CAUUUUUUUG-5′ (SEQ ID NO:1) to 3′-CCUUUUUUUG-5′ (SEQ ID NO:2). The replication of phi-6 segment-L can be increased up to 5-fold by modifying the 3′ end of the positive-strand RNA (herein referred to as “(+ve)-RNA”) strand from 5′-CUCUCUCUCU-3′ (SEQ ID NO:3) to 5′-CUCUCUUCCC-3′ (SEQ ID NO:4). Similarly, the replication of phi-6 segment-M can be increased up to 5-fold by modifying the 3′ end of the (+ve)-RNA strand from 5′-CUCUCUCUCU-3′ (SEQ ID NO:3) to 5′-CUCUCUUCCC-3′ (SEQ ID NO:4). Furthermore, the replication of phi-6 segment-S can be increased up to 5-fold by modifying the 3′ end of the (+ve)-RNA strand from 5′-CUCUCUCUCU-3′ (SEQ ID NO:3) to 5′-CUCUCUUCCC-3′ (SEQ ID NO:4). Surprisingly, when used in combination, the effects of said altered termini are synergistic and the copy number of capsids rdsRP/rdsRN per bacterial genome is increased as much as 25-100-fold. The above has not been explored heretofore, since the main thrust of research focuses on the regulation of phage replication and transcription, and such dramatic changes to the replication and/or transcription rates would not be predicted to produce viable phage.

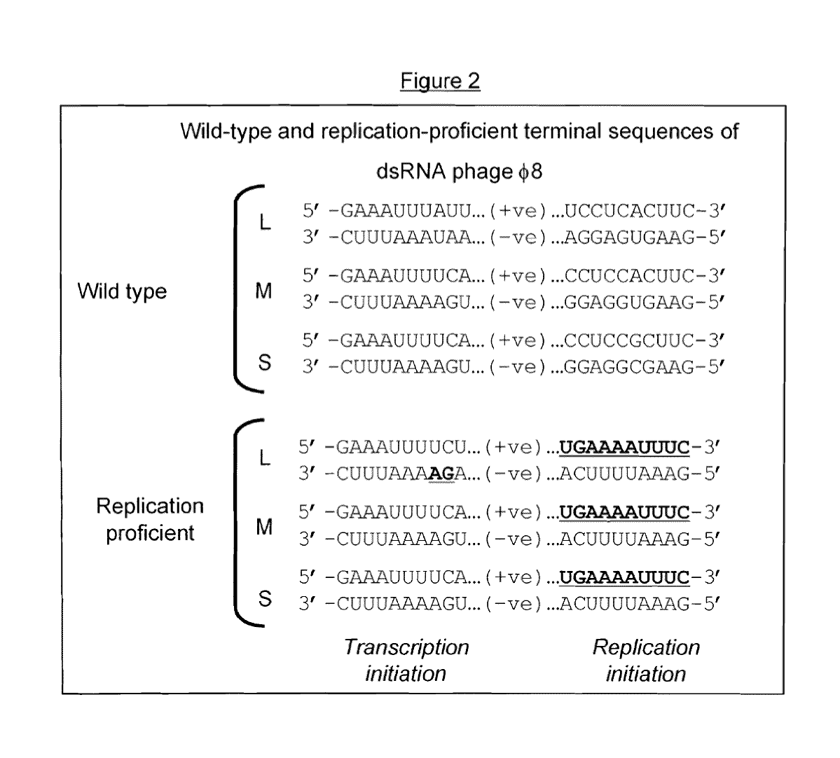

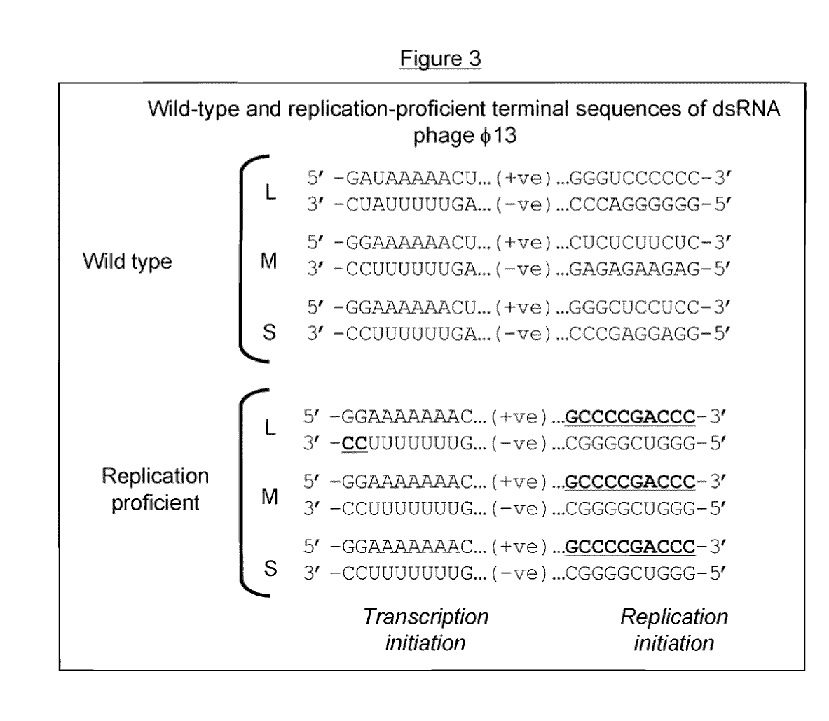

FIGS. 2 and 3 show possible changes to the dsRNA termini of phi-8 and phi-13 which can result in similar rdsRP/rdsRN copy number gains. As above, the changes can be used alone or in combination.

To ensure that the correct replication proficient ends are produced in RPCs, cDNA templates with the precise ends are used to generate mRNA encoding the desired recombinant segment. In addition, a promoter for in vitro transcription is included at the 5′ end of the cDNA template, which enables transcription to initiate at the desired initiation point to generate the appropriate 5′ end in the resulting mRNA molecules. Such DNA templates can be generated by recombinant DNA techniques, preferably using PCR, wherein the forward and reverse PCR primers generate the desired 5′ and 3′ ends in the resulting cDNA template. The cDNA templates can be generated with an SP6 promoter sequence (e.g. TTCTATAGTGTCACCTAAAT, SEQ ID NO:5) and the 5′ end, since SP6 DNA-dependent RNA polymerase, which is present in commercially available in vitro transcription kits, such as the Durascribe® SP6 transcription kit (Epicentre, Madison, Wis.) and MEGAscript® SP6 transcription kit (Ambion, Austin, Tex., Cat No. 1330), generates mRNA with the desired 5′ sequence found in RPCs of the present invention.

As an alternative strategy, cDNA templates encoding a promoter (such as SP6 for example) and a recombinant segment can be introduced directly into a target bacterial host (e.g. Sagawa et al., Gene 168:37; 1996) to enable transient expression of the recombinant segment with replication proficient ends. By including a template encoding segment-L, which will produce the procapsids in the target host strain, the transiently expressed recombinant segment(s) will be packaged and converted to dsRNA and in doing so, RPCs will be created and begin to replicate. cDNA templates in which thiolated nucleotides have been incorporated into the 3′ ends of the dsDNA will be more stable under such conditions and thus more efficient for launching RPCs; however, such thiolated sequences are not required to launch the RPCs.

Although the changes described above that result in RPC replication have been described in detail above, those skilled in the art will be capable of identifying alternative changes that result in increased capsid copy number per host genome and therefore do not deviate significantly from the spirit of this invention. For example, it is possible to use site directed or random mutagenesis procedures and selection strategies that produce RPCs. As discussed above, one selection strategy might entail using increasing levels of antibiotic selection, so as to exceed the level of resistance afforded by an rdsRP/rdsRP-encoded antibiotic-resistance gene unless the expression of said gene is augmented due to genetic changes that increase translation or replication rates and hence, increase the capsid copy number per host genome, thereby generating an RPC.

It is important to note that such increases in capsid copy number in bacterial hosts have not been reported heretofore. Furthermore, the synergistic effect of using more than one modified dsRNA terminal in RPC while maintaining the ability to replicate and without loss of stability is surprising and not predicted by the prior art. The usefulness of replication proficiency has been delineated above.

The replication rate and hence the copy number of capsids in host bacteria can be further enhanced by augmenting the level of segment-L expression in the host bacteria, since expression of segment-L leads to increased RNA-dependent RNA polymerase availability and hence, increased production of capsids. Increased segment-L expression can be accomplished by introducing a recombinant plasmid that encodes a prokaryotic expression cassette which encodes segment-L. Alternatively, sequences encoding segment-L can be integrated into the chromosome using procedures known to the art (Hone et al., Microbial Path. 5: 407; 1989; Hamilton et al., J. Bacteriol. 171: 4617; 1989; Blomfield et al., Mol. Microbiol. 5: 1447; 1991), under the control of a prokaryotic promoter, such as, but not limited to, the lac, trc, araBAD (i.e. P_{BAD}), and lambda P_{L} promoters. The location of chromosomal integration is not important to the present invention. In a preferred embodiment, plasmid-encoded or chromosomally-integrated segment-L sequences lack the 5-prime pac sequence, and thus are capable of procapsid expression but are not packaged into the capsids. That embodiment results in RPCs that are capable of harboring more rdsRNA since the space normally occupied by segment-L is now available for more passenger dsRNA.

Another rate limiting step to capsid production is the addition of P8. Thus, further useful compositions of the present invention comprise in vivo and in vitro systems that increase P8 expression. As above, increased P8 expression can be accomplished by introducing a recombinant plasmid that encodes a prokaryotic expression cassette that encodes, for example, a promoter, a ribosome-binding site, the P8 gene and a transcription terminator. Alternatively, sequences encoding, for example, a promoter, a ribosome-binding site, the P8 gene and a transcription terminator can be integrated into a chromosome using procedures known to the art (Hone et al., Microbial Path 5: 407; 1989; Hamilton et al., J. Bacteriol. 171: 4617; 1989; Blomfield et al., Mol. Microbiol. 5: 1447; 1991). P8 expression in such circumstances can be placed under the control of a prokaryotic promoter, such as, but not limited to, the lac, trc, araBAD (i.e. P_{BAD}), and lambda P_{L} promoters.

Note, since the yield of capsids is critical at the final stage of the host strain culture, the expression of segment-L and or P8 can be placed under the control of a regulated promoter, such as the araBAD (GenBank Accession No. K00953) or Tet promoter (GenBank Accession No. X00006), and can be activated late in the fermentation process to prevent over-production of capsids causing toxic effects on the host bacterial strain.

The particular dsRP from which segment-L is obtained is not critical to the present invention and includes, but is not restricted to, one of Phi-6 Segment-L (Genbank Accession No. M17461), Phi-13 Segment-L (Genbank Accession No. AF261668), or Phi-8 Segment-L (Genbank Accession No. AF226851), and are available from Dr. L. Mindich at Department of Microbiology, Public Health Research Institute, NY, N.Y. Alternatively, a recombinant segment-L can be made synthetically by any qualified commercial vendor, such as, DNA 2.0 Inc. (Menlo Park, Calif.), Blue Heron Biotechnology (Bothell, Wash.), Geneart Inc. (Toronto Ont, Canada), and Genscript Inc. (Piscataway, N.J.).

### Expression Cassettes

The prior art only suggests that rdsRP/rdsRN are useful for expression in mammalian cells but does not provide enabling compositions and methods thereto. In contrast, the present invention provides RPCs that are useful for expression of, for example, carbohydrates, lipids, proteins and/or trait modifying RNA sequences in, for example, bacteria, yeast, plants and mammals. In a preferred approach, said RPCs comprise dsRNA encoding a 5′ replication-initiation sequence containing the modified replication proficient terminal, a ribosomal binding site (for expression in bacteria) or 5′-translation loop formation sequence (for expression in yeast, plants and mammalian cells), one or more translation enhancer sequences, such as a Shine-Delgarno (infra) or a Kozak sequence (infra), at least one gene of interest or trait modifying sequence of interest, a 3′-translation loop formation sequence; (for expression in yeast, plants and mammalian cells), and a modified 3′ transcription-initiation sequence containing a transcription-proficient sequence.

### Bacterial Expression Cassettes

The prior art, U.S. Pat. No. 7,018,835 and US Publ. No. 20060115493, do not provide compositions or methods for expression of recombinant proteins or trait modifying RNA sequences in bacteria. On the other hand, an embodiment of the present invention has been met by providing RPCs that are capable of expressing recombinant proteins and/or trait modifying RNA sequences in bacteria. Bacterial expression cassettes are comprised of a ribosome-binding site (herein referred to as “RBS”) followed by a gene of interest. The particular RBS is not important to the present invention and is selected as the optimal RBS for expression in the target bacterial strain. An extensive description of bacterial RBS is found elsewhere (Shine and Dalgarno, Nature 254:34; 1975; Gualerzi and Pon, Biochem. 29:5881; 1990). Any of a variety of genes can be expressed in bacteria as known in the art, see general references cited herein, and the particular gene to be expressed in a design choice.

As mentioned, RPCs are also capable of expressing trait modifying RNA sequences in bacteria. Examples of trait modifying RNA sequences include, but are not limited to, those that inactivate auxotrophic genes, such as, but not limited to, aro (Hoiseth et al., Nature, 291:238-239 (1981)), qua (McFarland et al., Microbiol. Path., 3:129-141 (1987)), nad (Park et al., J. Bact., 170:3725-3730 (1988), thy (Nnalue et al., Infect. Immun., 55:955-962 (1987)), and asd (Curtiss, Infect. Immun, 55:3035-3043 (1987)) genes; those that inactivate global regulatory functions, such as cya (Curtiss et al., Infect. Immun., 55:3035-3043 (1987)), crp (Curtiss et al. (1987), supra), phoP/phoQ ((Groisman et al., Proc. Natl. Acad. Sci., USA, 86:7077-7081 (1989); and Miller et al., Proc. Natl. Acad. Sci., USA, 86:5054-5058 (1989)), or ompR (Dorman et al., Infect. Immun, 57:2136-2140 (1989)) genes; those that modify the stress response, such as recA (Buchmeier et al., Mol. Micro., 7:933-936 (1993)), htrA (Johnson et al., Mol. Micro., 5:401-407 (1991)), htpR (Neidhardt et al., Biochem. Biophys. Res. Com., 100:894-900 (1981)), hsp (Neidhardt et al., Ann Rev. Genet., 18:295-329 (1984)) and groEL (Buchmeier et al., Sci., 248:730-732 (1990)) genes; those that inactivate specific virulence factors, such as lsyA (Libby et al., Proc. Natl. Acad. Sci., USA, 91:489-493 (1994)), pag or prg (Miller et al (1989), supra), iscA or virG (d'Hauteville et al., Mol. Micro., 6:833-841 (1992)), plcA (Mengaud et al., Mol. Microbiol., 5:367-72 (1991) and Camilli et al, J. Exp. Med, 173:751-754 (1991)), and act (Brundage et al., Proc. Natl. Acad. Sci., USA, 90:11890-11894 (1993)) genes; those that inactivate genes involved in DNA topology, such as topA (Galan et al., Infect. Immun., 58:1879-1885 (1990)) genes; those that inactivate biogenesis of surface polysaccharides, such as rfb, galE (Hone et al., J. Infect. Dis., 156:164-167 (1987)) or via (Popoff et al., J. Gen. Microbiol., 138:297-304 (1992)) genes; those that inactivate suicide systems, such as sacB (Recorbet et al., App. Environ. Micro., 59:1361-1366 (1993) and Quandt et al., Gene, 127:15-21 (1993)), nuc (Ahrenholtz et al., App. Environ. Micro., 60:3746-3751 (1994)), hok, gef, kil, or phlA (Molin et al., Ann Rev. Microbiol., 47:139-166 (1993)) genes; those that introduce suicide systems, such as lysogens encoded by P22 (Rennell et al., Virol., 143:280-289 (1985)), lambda murein transglycosylase (Bienkowska-Szewczyk et al., Mol. Gen. Genet., 184:111-114 (1981)) or S-gene (Reader et al., Virol., 43:623-628 (1971)); those that disrupt or modify the cell cycle, such as, minC, minD or minE (de Boer et al., Cell, 56:641-649 (1989)) genes; and so on.

### Translation Loop Sequences

Translation loop sequences (herein “referred to as “TLS”) are unique to the present invention and are not described in the context of dsRP. TLS are distinguished from an internal ribosome entry site (i.e. IRES) and a cap-independent translation enhancer (i.e. CITE) in that TLS are comprised of two elements, a 5′ end and a 3′ end which result in a message being looped for translation by a ribosome. The particular sequences at the 5′ end and the 3′ end of the mRNA are not critical to the instant invention and to the phenomenon of having the translated 5′ end of a message being brought into proximity of the site on the ribosome where the mRNA is translated. Thus, proteins that bind to nucleic acid can mediate the looping of an mRNA to enable retranslation of the same message. However, other means and molecules can cause the repeated translation of a message, and is not restricted two proteins, nucleic acid sequences at the 5′ and 3′ ends and so, so long as a message is translated multiply and efficiently. For example, the 5′ end of a TLS can encode sequences for, for example, elongation factor-3 binding, a 40s ribosome subunit recognition sequence and sequences that bind to a polypyrimidine tract binding protein complexed with the second element, which typically is at the 3′ end and can encode, for example, a combined polypyrimidine tract binding protein recognition sequence (Edgil & Harris, Virus Res., 119:43; 2006). U.S. Pat. No. 7,018,835 and US Publ. No. 20060115493 do not include these elements and are unlikely to effect translation in mammalian cells due to this omission. A surprising finding of this invention is that TLS are required to produce rdsRP, rdsRN and RPCs that are capable of expression in host cells and merit commercial development as in vitro and in vivo expression vectors. As such, the use of TLS has unanticipated utility both as an improvement to the rdsRP and rdsRN technologies described in U.S. Pat. No. 7,018,835 and US Publ. No. 20060115493, respectively, as well as enabling novel RPC compositions in the present invention. The particular rdsRP and rdsRN is not important to the present invention and include those described in U.S. Pat. No. 7,018,835 and US Publ. No. 20060115493, respectively, except that the TLS described herein replace the dysfunctional CITE and IRES sequences.

### Yeast Expression Cassettes

U.S. Pat. No. 7,018,835 and US Publ. No. 20060115493 do not provide, suggest or allude to compositions or methods for expression of recombinant proteins or trait modifying RNA sequences in yeast. On the other hand, an embodiment of the present invention has been met by providing dsRP, dsRN and RPCs that are capable of expressing a recombinant protein and/or a trait modifying RNA sequence in yeast. For efficient expression of the recombinant protein of interest, yeast expression cassettes are comprised of a TLS in the 5′ and 3′ ends of the rdsRNA segment. The particular TLS useful for expression of recombinant proteins in yeast is not important to the present invention and include, but are not limited to, the TLS located at the 5′ and 3′ ends of the YAP1 and p150 genes of Saccharomyces cerevisiae (Zhou et al., Proc. Natl. Acad. Sci. USA 98: 1531-1536 (2001)), hepatitis C virus (Rosenfeld and Racaniello, J Virol 79: 10126-10137 (2005)), and cricket paralysis virus (Thompson et al., Proc. Natl. Acad. Sci. USA 98: 12972-12977 (2001)).

The 3′-component of TLS in (+ve)-RNA virus genomes, such as, hepatitis C virus (herein referred to as “HCV”), is often conserved and typically consists of a 30-40 nucleotide variable region and a 20-200 nucleotide homopolymeric poly-uridine (pU)/polypyrimidine (pP) tract followed by a 98 nucleotide 3× region, which is highly conserved across all serotypes (Chambers et al., Ann. Rev. Microbiol., 44:649; 1990). Chemical and biochemical studies together with modeling (MFOLD) suggest that the 3× region consists of three stem-loops. In chimpanzees, both the pU/pP and the 3× region are necessary for viral infection and replication, whereas the variable region appears to be dispensable (Yanagi et al., Proc. Natl. Acad. Sci., 96:2291; 1999; Kolykhalov et al., J. Virol., 74:2046; 2000). Translational efficiency is affected by a synergistic interaction between the 5′ and 3′ ends of the mRNA, which can involve a physical link between the 5′ and 3′ ends via protein and/or RNA contacts, resulting in formation of the translation loop. Although there can be regions of sequence complementarity in the 5′ and 3′ ends of the TLS, RNA-RNA interactions are not required.

There is evidence showing that loop formation can also involve protein-RNA interactions (Yu et al., J. Virol., 73:3638; 1999; Kieft et al. Cold Spring Harbor Symposia on Quantitative Biology, Cold Spring Harbor Laboratory Press, Vol. 66, pp 277; 2001). Proteins that bind the 3′-component of the TLS include La (Spangberg et al., J. Gen. Virol., 82:113; 2001), hnRNP-C (Gontarek et al., Nucl. Acid Res., 27:1457; 1999), HuR (Spangberg et al., Virol., 274:378; 2000), glyceraldehyde 3-phosphate dehydrogenase (Petrik et al., J. Gen. Virol., 80:3109; 1999), viral protein NS3 (Banerjee and Dasgupta, J. Virol., 75:1708; 2001), ribosomal protein L22 (Wood et al. 2001), and pyrimidine tract binding protein (herein “PTBP”; Ito and Lai, J. Virol., 71:8698; 1997; Tsuchihara et al., J. Virol., 71:6720; 1997; and Chung and Kaplan, Biochem. Biophys. Res. Commun, 254:351; 1999). For example, PTBP appears to interact with stem-loops SL2 and SL3 in the 3× region and the integrity of the stem-loops plays an important role in that interaction (Ito and Lai, supra; 1997; Chung and Kaplan, supra; 1999).

Given the importance of PTBP in the interaction with the 3′-component of the TLS, an assay can be developed to identify sequences that interact with purified PTBP. Purification of PTBP can be achieved by expressing a recombinant form of PTBP containing a histidine tag in E. coli and purification of the PTBP_{His} by affinity chromatography using a His tag purification kit (Invitrogen Inc., Carlsbad, Calif.). PTBP binding to RNA sequences can be evaluated using a Pierce (Rockford, Ill.) LightShift electrophoresis mobility shift assay according to the instructions of the manufacturer.

### Plant Expression Cassettes

As mentioned, U.S. Pat. No. 7,018,835 and US Publ. No. 20060115493 do not provide, suggest or allude to compositions or methods for expression of recombinant proteins or trait modifying RNA sequences in plants. On the other hand, an objective of the present invention is to provide rdsRP, rdsRN and RPCs that are capable of expressing a recombinant protein and/or a trait modifying RNA sequence in plants.

For efficient expression of a recombinant protein of interest, plant expression cassettes are comprised of a TLS in the 5′ end and the 3′ end of the rdsRNA segment (reviewed in Dreher and Miller, Virol. 344:185; 2006). For example, the 5′-end of the 3′-UTR of barley yellow dwarf luteovirus (BYDV) RNA carries about a 100 base sequence that facilitates highly efficient translation initiation at the 5′-proximal AUG of the mRNA (Guo et al., RNA, 6:1808; 2000; Wang et al., EMBO, 16:4107; 1997). That BYDV-like TLS is conserved in all luteoviruses and in the dianthovirus (Mizumoto et al., J. Virol., 77:12113; 2003) and necrovirus (Meulewaeter et al., Nucl. Acid Res., 32:1721; 2004; Shen and Miller, J. Virol., 78:4655; 2004) genera of the Tombusviridae. The BYDV-like TLS BTE is defined by a 17-nucleotide conserved sequence, GGAUCCUGGGAAACAGG (SEQ ID NO:6), that forms a stem-loop and by at least one additional stem-loop, whose loop base-pairs to the 5′-UTR (Guo et al., Mol. Cell. 7:1103; 2001). A tract in that element also has potential to base-pair near the 3′-end of 18S rRNA (Wang et al., supra, 1997). That may contribute to recruitment and recycling of the ribosomes to and within the TLS. However, the particular TLS useful for expression of recombinant proteins in plants is not important to the present invention and includes, but is not limited to, the TLS located in the crucifer-infecting tobamovirus (Ivanov et al., Virology 232: 32; 1997; Skulachev et al., Virology 263: 139; 1999; Dorokhov et al., J. Gen. Virol., 87: 2693; 2006)), tomato bushy stunt virus (Monkewich et al., J. Virol., 79:4848; 2005), maize Hsp 101 gene (Dinkova et al., Plant J., 41:722; 2005), potato leafroll polerovirus (Jaag et al., Proc Natl Acad Sci USA 100:8939; 2003), and Rhopalosiphum padi virus (Domier et al, Virol., 268: 264; 2000; Woolaway et al., J Virol 75:10244; 2001).

### Mammalian Expression Cassettes

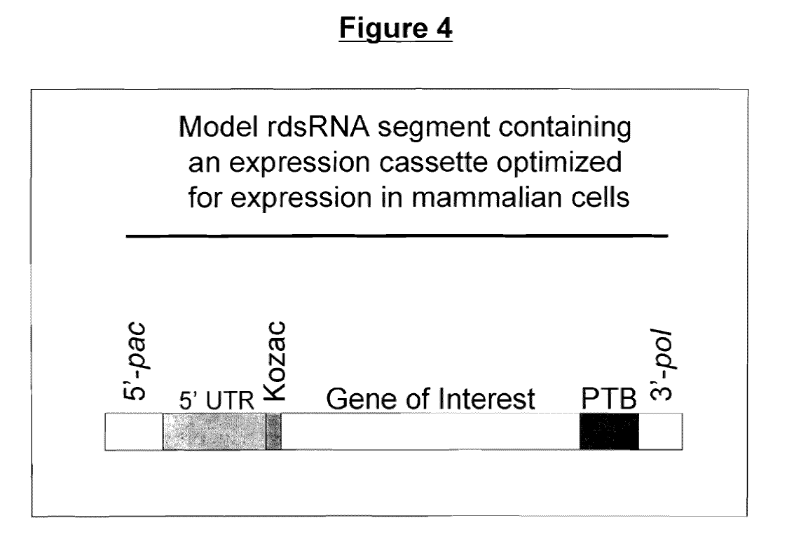

The design of prototype expression cassettes capable of, at best, low-level expression of at least one gene of interest in target mammalian cells is described elsewhere (U.S. Pat. No. 7,018,835 and US Publ. No. 20060115493). In addition, the present invention provides rdsRP, rdsRN and RPCs that express a recombinant protein of interest in mammalian cells through the novel utilization of expression cassettes that contain TLS (Dreher and Miller, Virol. 344:185; 2006) at the 5′ and 3′ ends of the gene of interest in the recombinant dsRNA segment. Generally speaking, recombinant dsRNA segments carrying expression cassettes are configured as shown in FIG. 4. The particular TLS is not important in the practice of the present invention and includes, but is not limited to, the 5′- and 3′-UTR of the hepatitis C virus (herein referred to as “HCV”). It is important to note that the 3′ UTR of the TLS in HCV is comprised of a polypyrimidine sequence that is recognized by the polypyrimidine tract binding protein (herein referred to as “PTBP”) and does not contain a polyadenosine (also know as “poly-A”) sequence. In fact, the use of polyadenosine, as described in (U.S. Pat. No. 7,018,835 and US Publ. No. 20060115493), impedes the binding of PTBP in mammalian cells. Thus, an object of the present invention has been met by providing novel rdsRP and rdsRN expression cassettes that are capable of efficient expression of recombinant proteins in mammalian cells.

Expression of genes of interest can also be achieved using rdsRP, rdsRN and RPCs that contain at least one “Kozak” sequences adjacent to the translation start codon (e.g. Kozak, J. Mol. Biol. 196:947-950; 1987). Preferably, rdsRP, rdsRN and RPCs contain two to twenty Kozak sequences. In one embodiment, rdsRP, rdsRN and RPCs contain 5-15 Kozak sequences. Alternatively, expression cassettes can contain a 5′- and 3′ TLS, and at least one Kozak sequence functionally linked to the 5′-TLS. As shown in the examples below, genes of interest in rdsRP, sdsRN and RPCs are expressed at impressive levels in mammalian cells by employing such configurations.

As a further alternative, translation of recombinant proteins encoded in rdsRP, rdsRN and RPCs can be achieved by incorporating at least one untranslated region (herein referred to as “UTR”) sequence such as the 3′-UTR of the mammalian β-catalytic subunit of H⁺-ATP synthase mRNA (e.g. Izquierdo & Cuezva, Biochem J., 346:849-855; 2000), which direct translation of the upstream mRNA sequences. That strategy is particularly flexible, in that it enables both monocistronic and bicistronic expression, since these novel UTR sequences promote translation of sequences from both upstream and downstream locations. As such, the use of UTR sequences has unanticipated utility both as an improvement to the rdsRP and rdsRN technologies described in U.S. Pat. No. 7,018,835 and US Publ. No. 20060115493, respectively, as well as enabling expression by the novel RPC compositions in the present invention.

The particular UTR is not important to the present invention and includes, but is not restricted to, the 3′-UTR of the (3 subunit of human mitochondrial ATPase (GenBank Accession no. M57634), and the 3′-UTR of subunit IV of cytochrome c oxidase (Accession number X54081). Alternatively, the 5′-UTR of mouse Gtx homeodomain protein has similar activity and can be used in an analogous manner (Hu et al., Proc Natl Acad Sci USA 96:1339 (1999)).

Surprisingly, furthermore, such UTR sequences can be used in combination with 5′-CITE sequences in rdsRP described in U.S. Pat. No. 7,018,835 and in combination with 5′-IRES sequences in rdsRN described in US Patent Application no. 20060115493, to significantly enable expression of genes of interest in target mammalian cells. In a yet another embodiment, the aforementioned UTR sequences are used in combination with TLS to further enhance translation of recombinant proteins in RPCs of the present invention.

In addition, rdsRP, rdsRN and RPCs containing a UTR sequence can also contain at least one “kozak” sequence adjacent to the translation start codon (e.g. Kozak, J. Mol. Biol. 196:947-950; 1987). Preferably, expression cassettes contain TLS, and a Kozak sequence functionally linked to the 5′-TLS and a UTR (e.g. 5′-TLS::Kozak::gene of interest::3′-TLS::UTR, wherein “::” denotes a novel junction). Such a configuration enables high-level expression of genes of interest by rdsRP, rdsRN and RPCs in mammalian cells.

In another embodiment, a TLS contains sequences that inhibit host anti-viral cellular defense mechanisms, such as the components of the dsRNA response pathway (Vyas et al. RNA 9:858-870; (2003); (McKenna et al., J Mol Biol 358:1270-1285 (2006); and McKenna et al., J Mol Biol 372:103-113; (2007)). Fortuitously, the HCV TLS contains sequences that inhibit double stranded RNA-activated protein kinase (PKR), endowing that sequence with a dual function of promoting translation and inhibiting PKR (Vyas et al. RNA 9:858-870; (2003)). Sequences with similar function are found in other human, plant, avian, fungal, reptilian, insect, fish and mollusk dsRNA viruses (Mertens, Virus Research 101:3-13 (2004)). Other sequences that are known to inhibit PKR include, but are not limited to, EBERT from Epstein-Barr virus and VAI from adenovirus (McKenna et al., J Mol Biol 358:1270-1285 (2006); and McKenna et al., J Mol Biol 372:103-113; (2007)). Furthermore, any nucleotide sequence that inhibits self-association and autophosphorylation of PKR can be included in the dsRNA segment to inhibit PKR (McKenna et al., J Mol Biol 372:103-113; (2007)). An assay can be devised wherein RPCs containing a reporter gene flanked by TLS sequences that do not contain a PKR inhibitory motif and a segment that contains a library of putative PKR-inhibitory RNA sequences is introduced into a host cell. Expression of the reporter gene denotes the presences of the PKR-inhibitory sequence, which can be rescued by cell sorting and RT-PCR methods well know in the art. Embellishment of the translation-promoting, PKR-inhibiting strategy can be achieved by producing RPCs that express proteins capable of inhibiting PKR, such as, but not limited to, proteins that target Cardif, such as the HCV protease NS3 described herein, NS3-4a (Meylan et al., Nature 437:1167-1172 (2005)), NSSa, the C-terminal region of NS5A-1b and related proteins (Pflugheber et al., Proc Natl Acad Sci USA 99:4650-4655; (2002); and Noguchi et al., Microbiol. Immunol. 45:829-840; (2001)), etc.

As a further remedy to innate host defense, it is important to ensure that sequences encoded within the rdsRP, RdsRN and RPCs of the present invention do not contain sequences that are targets of RNases and gene silencing RNA and microRNA sequences. Genomic tools available at the National Library of Medicine) and at TIGR, now known as The Craig Venter Institute, provide resources to enable rdsRP, rdsRN and RPC designers to ensure such sequences are not included.

Solutions to innate host defenses, such as PKR and inhibitory RNA, can be borrowed from plant viruses and applied to expression of genes and trait modifying RNA sequences in plants (e.g. Wang & Metzlaff, Curr. Opinion in Plant Biol. 8:216 (2005)).

### Construction of Expression Cassettes

Expression cassettes can be made synthetically by any qualified commercial vendor, such as DNA 2.0 Inc. (Menlo Park, Calif.), Blue Heron Biotechnology (Bothell, Wash.), Geneart Inc. (Toronto Ont, Canada), and Genscript Inc. (Piscataway, N.J.). To synthesize expression cassettes, a series of short sequences 100-200 base pairs in length are generated and ligated together to form the full-length sequence using procedures well know in the art (Ausubel et al., supra, 1990). The synthetic DNA is produced using an Applied Biosystems International ABI™ 3900 High-Throughput DNA Synthesizer (Foster City, Calif.) using procedures provided by the manufacturer.

### Genes of Interest

As described above, the expression cassette is capable of expressing genes of interest in the host cell. Genes of interest can be any gene that encodes any protein or parts thereof in the target host cell. Genes of interest can also encode attenuated viruses and virus-like particles; the latter can be engineered to disseminate throughout the host and deliver either transient or stable phenotypic traits to host cells (e.g. Marillonnet et al., Proc. Natl. Acad. Sci. (USA), 101:6857; 2004). In some instances the gene of interest encodes vaccine antigens, can be any molecule that is expressed by any viral, bacterial, or parasitic pathogen prior to or during entry into, colonization of, or replication in their animal host; the novel rdsRP and rdsRN, as well as the RPCs of the present invention may express vaccine antigens or parts thereof that originate from viral, bacterial and parasitic pathogens and so on. The pathogens can be infectious in humans, domestic animals or wild animal hosts.

“Immunogen” and “antigen” are used interchangeably herein as a molecule that elicits a specific immune response containing an antibody that binds to that molecule. That molecule can contain one or more sites to which a specific antibody binds. As known in the art, such sites are known as epitopes. Thus, an RPC can express an immunogen that can be used to raise an antibody response in a host.

A vaccine is an immunogen used to generate an immunoprotective response. The dosage is derived, extrapolated and/or determined from preclinical and clinical studies, as known in the art. Multiple doses can be administered as known in the art, and as needed to ensure a prolonged prophylactic state. The successful endpoint of the utility of a vaccine for the purpose of this invention is the resulting presence of an induced serum antibody, or antibody made by the host in any tissue or organ, that binds the glycoprotein immunogen of interest, and perhaps preferably a high mannose epitope. In some embodiments, the induced antibody in some way, neutralizes and/or eliminates the pathogen carrying the cognate high mannose glycoprotein of interest. For the purposes of the instant invention, observing immunoprotection of at least thirty days is evidence of efficacy of a vaccine of interest. The time of immunoprotection can be at least 45 days, at least 60 days, at least 3 months, at least 4 months, at least 5 months, at least 6 months, at least 1 year, at least 2 years or longer. Preferably the immunoprotection is observed in outbred populations, and to different forms, strains, variants, alleles and the like of a pathogen.

The viral pathogens from which the viral antigens are derived, include, but are not limited to, Orthomyxoviruses, such as influenza virus (Taxonomy ID: 59771); Retroviruses, such as RSV, HTLV-1 (Taxonomy ID: 39015), and HTLV-II (Taxonomy ID: 11909), Papillomaviridae, such as HPV (Taxonomy ID: 337043), Herpesviruses, such as EBV (Taxonomy ID: 10295); CMV (Taxonomy ID: 10358); or herpes simplex virus (ATCC No.: VR 1487); Lentiviruses, such as HIV-1 (Taxonomy ID: 12721) and HIV-2 (Taxonomy ID: 11709); Rhabdoviruses, such as rabies; Picornoviruses, such as Poliovirus (Taxonomy ID: 12080); Poxviruses, such as vaccinia (Taxonomy ID: 10245); Rotavirus (Taxonomy ID: 10912); and Parvoviruses, such as adeno-associated virus 1 (Taxonomy ID: 85106).

Examples of viral antigen genes can be found in the group including, but not limited to, the human immunodeficiency virus antigens, nef (National Institute of Allergy and Infectious Disease HIV Repository Cat. No. 183; Genbank Accession no. AF238278), gag, env (National Institute of Allergy and Infectious Disease HIV Repository Cat. No. 183 and No. 2433; Genbank Accession No. AF238278), gag, env (No. U39362), tat (National Institute of Allergy and Infectious Disease HIV Repository Cat. No. 2433; Genbank Accession No. U39362), tat (National Institute of Allergy and Infectious Disease HIV Repository Cat. No. 827; Genbank Accession No. M113137), mutant derivatives of tat, such as tatΔ31-45 (Agwale et al., Proc. Natl. Acad. Sci. USA 99:10037; 2002), rev (National Institute of Allergy and Infectious Disease HIV Repository Cat. No. 2088; Genbank Accession No. L14572), and pol (National Institute of Allergy and Infectious Disease HIV Repository Cat. No. 238; Genbank Accession No. AJ237568) and T and B cell epitopes of gp120 (Hanke and McMichael, AIDS Immunol Lett., 66:177; 1999; Hanke, et al., Vaccine, 17:589; 1999; Palker et al., J. Immunol., 142:3612-3619; 1989), chimeric derivatives of HIV-1 env and gp120, such as, but not restricted to, fusion between gp120 and CD4 (Fouts et al., J. Virol. 2000, 74:11427-11436; 2000); truncated or modified derivatives of HIV-1 env, such as, but not restricted to, gp140 (Stamatos et al., J Virol, 72:9656-9667; 1998) or derivatives of HIV-1 env and/or gp140 thereof (Binley et al., J Virol, 76:2606-2616; 2002; Sanders et al., J Virol, 74:5091-5100; 2000; Binley et al. J Virol, 74:627-643; 2000), the hepatitis B surface antigen (Genbank Accession No. AF043578; Wu et al., Proc. Natl. Acad. Sci., USA, 86:4726-4730; 1989); rotavirus antigens, such as VP4 (Genbank Accession No. AJ293721); (Mackow et al., Proc. Natl. Acad. Sci., USA, 87:518-522; 1990)) and VP7 (GenBank Accession No. AY003871); (Green et al., J. Virol., 62:1819-1823; 1988)), bacteriophage phi-8 segment S sequence (GenBank Accession Nos. AF226852 and 226853); hepatitis C sequences, such as GenBank Accession No. AJ242651; influenza virus antigens, such as, hemagglutinin (GenBank Accession No. AJ404627 and (Pertmer and Robinson, Virology, 257:406; 1999) or nucleoprotein (GenBank Accession No. AJ289872 and Lin et al., Proc. Natl. Acad. Sci., 97: 9654-9658; 2000) and herpes simplex virus antigens, such as, thymidine kinase (Genbank Accession No. AB047378 and Whitley et al., In: New Generation Vaccines, pages 825-854).

The bacterial pathogens, from which the bacterial antigens are derived, include, but are not limited to: Mycobacterium spp., Helicobacter pylori, Salmonella spp., Shigella spp., E. coli, Rickettsia spp., Listeria spp., Legionella pneumoniae, Pseudomonas spp., Vibrio spp., Bacillus anthracis and Borellia burgdorferi.

Examples of protective antigens of bacterial pathogens include the somatic antigens of enterotoxigenic E. coli, such as the CFA/I fimbrial antigen (Yamamoto et al., Infect. Immun., 50:925-928; 1985) and the nontoxic B-subunit of the heat-labile toxin (Klipstein et al., Infect. Immun., 40:888-893; 1983); pertactin of Bordetella pertussis (Roberts et al., Vacc., 10:43-48; 1992), adenylate cyclase-hemolysin of B. pertussis (Guiso et al., Micro. Path., 11:423-431; 1991), fragment C of tetanus toxin of Clostridium tetani (Fairweather et al., Infect. Immun., 58:1323-1326; 1990), OspA of Borellia burgdorferi (Sikand et al., Pediatrics, 108:123-128; 2001); (Wallich et al., Infect Immun, 69:2130-2136; 2001)), protective paracrystalline-surface-layer proteins of Rickettsia prowazekii and Rickettsia typhi (Carl et al., Proc Natl Acad Sci USA, 87:8237-8241; 1990), the listeriolysin (also known as “Llo” and “Hly”) and/or the superoxide dismutase (also know as “SOD” and “p60”) of Listeria monocytogenes (Hess et al., Infect. Immun 65:1286-92; 1997); Hess et al., Proc. Natl. Acad. Sci. 93:1458-1463; 1996; Bouwer et al., J. Exp. Med. 175:1467-71; 1992)), the urease of Helicobacter pylori (Gomez-Duarte et al., Vaccine 16, 460-71; 1998; Corthesy-Theulaz, et al., Infection & Immunity 66, 581-6; 1998)), and the Bacillus anthracis protective antigen and lethal factor receptor-binding domain (Price et al., Infect. Immun. 69, 4509-4515; 2001).

The parasitic pathogens, from which the parasitic antigens are derived, include, but are not limited to: Plasmodium spp., such as Plasmodium falciparum (ATCC No.: 30145); Trypanosome spp., such as Trypanosoma cruzi (ATCC No.: 50797); Giardia spp., such as Giardia intestinalis (ATCC No.: 30888D); Boophilus spp., Babesia spp., such as Babesia microti (ATCC No.: 30221); Entamoeba spp., such as Entamoeba histolytica (ATCC No: 30015); Eimeria spp., such as Eimeria maxima (ATCC No.: 40357); Leishmania spp. (Taxonomy ID: 38568); Schistosome spp., Brugia spp., Fascida spp., Dirofilaria spp., Wuchereria spp., and Onchocerea spp.

Examples of protective antigens of parasitic pathogens include the circumsporozoite antigens of Plasmodium spp. (Sadoff et al., Science, 240:336-337; 1988), such as the circumsporozoite antigen of P. bergerii or the circumsporozoite antigen of P. falciparum; the merozoite surface antigen of Plasmodium spp. (Spetzler et al., Int. J. Pept. Prot. Res., 43:351-358; 1994); the galactose specific lectin of Entamoeba histolytica (Mann et al., Proc. Natl. Acad. Sci., USA, 88:3248-3252; 1991), gp63 of Leishmania spp. (Russell et al., J. Immunol., 140:1274-1278; 1988; Xu & Liew, Immunol., 84: 173-176; 1995)), gp46 of Leishmania major (Handman et al., Vaccine, 18:3011-3017; 2000), paramyosin of Brugia malayi (Li et al., Mol. Biochem. Parasitol., 49:315-323; 1991), the triose-phosphate isomerase of Schistosoma mansoni (Shoemaker et al., Proc. Natl. Acad. Sci., USA, 89:1842-1846; 1992); the secreted globin-like protein of Trichostrongylus colubriformis (Frenkel et al., Mol. Biochem. Parasitol., 50:27-36; 1992); the glutathione-S-transferase of Frasciola hepatica (Hillyer et al., Exp. Parasitol., 75:176-186; 1992), Schistosoma bovis and S. japonicum (Bashir et al., Trop. Geog. Med., 46:255-258; 1994); and keyhole limpet hemocyanin (KLH)-cross-reactive molecules of Schistosoma bovis and S. japonicum (Bashir et al., supra, 1994).

As mentioned earlier, the RPC vaccine may encode an endogenous immunogen, which may be any cellular protein, immunoregulatory agent, or therapeutic agent, or parts thereof, that may be expressed in the recipient cell, including, but not limited to, tumor, transplantation, and autoimmune immunogens, or fragments and derivatives of tumor, transplantation, and autoimmune immunogens thereof. Thus, in the present invention, RPC may encode tumor, transplant, or autoimmune immunogens, or parts or derivatives thereof. Alternatively, the RPC may encode synthetic genes taught herein and as known in the art, including those which encode tumor-specific, transplant, or autoimmune antigens or parts thereof.

Examples of tumor-specific antigens include prostate-specific antigen (Gattuso et al., Human Pathol., 26:123-126; 1995), TAG-72 and CEA (Guadagni et al., Int. J. Biol. Markers, 9:53-60; 1994), MAGE-1 and tyrosinase (Coulie et al., J. Immunothera., 14:104-109; 1993). Recently it has been shown in mice that immunization with non-malignant cells expressing a tumor antigen provides a vaccine effect, and also helps the animal mount an immune response to clear malignant tumor cells displaying the same antigen (Koeppen et al., Anal. N.Y. Acad. Sci., 690:244-255; 1993).

Other tumor-specific antigens include, but are not limited to, epithelial cell mucin (e.g. muc-1; GenBank Accession No. U60259, NM_{—}001044390), ovarian carcinoma antigen (e.g. muc-16; GenBank Accession No. NM_{—}024690), placental alkaline phosphatase (GenBank Accession No. BC009647), estrogen receptor (GenBank Accession No. AA744644), oncofetal antigen-immature laminin receptor (GenBank Accession No. AF140348), melanoma-associated antigen p155 (Loop et al., Int. J. Cancer, 27:775; 1981), epidermal growth factor receptor (e.g. erythroblastic leukemia viral oncogene (v-erb-b), GenBank Accession No. NM 005228), prostate-specific antigen (GenBank Accession No. M26663), tumor-associated antigen-72 (e.g. TAG-72; Thor et al., Int. J. Cancer, 43:810; 1989), which is expressed on the surface of various human tumor cells, such as the colon adenocarcinoma cell line LS174T (ATCC No. CL188), a variant of cell line LS180 (ATCC No. CL187)), carcinoembryonic antigen (GenBank Accession No. AA280641), melanoma-associated antigen-E1 (e.g. MAGE-1a, GenBank Accession No. AB040527), MAGE-1b (GenBank Accession No. AB040528); MAGE-1c, (GenBank Accession No. AB040529), melanoma-associated antigen-E3 (e.g. MAGE-3, GenBank Accession No. T29746), tyrosinase (e.g. p97; GenBank Accession No. M63235, M60296, NM_{—}000372), etc.

Recently it has been shown that immunization of humans with tumor antigens provides protection against cancer progression, and elicits an immune response to clear malignant tumor cells displaying the same antigen (e.g. Liu et al., Proc. Natl. Acad. Sci., 101:14567; 2004; Nemunaitis, Expert Rev. Vaccine, 4:259; 2005; and North and Butts, Expert Rev. Vaccine, 4:249; 2005). Methods and vaccination strategies that employ tumor antigens to treat cancer in humans and veterinary animals are described (Gattuso et al., Human Pathol., 26:123-126; 1995; Ojima et al., Int. J. Cancer, 120:585; 2007; Hoon et al., J. Immunol., 154:730; 1995; Mishra and Sinha, J. Biomol. Strct. Dyn., 24:109; 2006; Koeppen et al., Anal. N.Y. Acad. Sci., 690:244; 1993; Loop et al., Int. J. Cancer, 27:775; 1981; Lottich et al., British Can. Res. Treat., 6:49; 1985; Coulie et al., J. Immunothera., 14:104; 1993; Guadagni et al., Int. J. Biol. Markers, 9:53; 1994; Atanackovic et al., J. Immunol., 172:3289; 2004; Zhang et al., Proc. Natl. Acad. Sci. (USA), 100:15101; 2003; Nemunaitis, Expert Rev. Vaccine, 4:259; 2005; Liu et al., Proc. Natl. Acad. Sci., 101:14567; 2004; and North and Butts, Expert Rev. Vaccine, 4:249; 2005).

Tumor antigens can be delivered alone, combined with RPCs that expresses an adjuvant, or combined with RPCs that expresses an immunostimulatory molecule, such as, interferon-γ (GenBank Accession No. X62470), granulocyte-monocyte colony stimulating factor (GenBank Accession No. X03021) or interleukin-2 (GenBank Accession No. U25676), for example.

Marker genes include the aminoglycoside phosphotransferase gene (DQ851853) which confers kanamycin resistance; the firefly luciferase gene (GenBank Accession No. DQ904455); and the chloramphenicol acetyltransferase gene (GenBank Accession No. AM295157), for example.

Examples of transplant antigens include the CD3 molecule on T cells (Alegre et al., Digest. Dis. Sci., 40:58-64; 1995). Treatment with an antibody to CD3 receptor has been shown to rapidly clear circulating T cells and reverse cell-mediated transplant rejection (Alegre et al., supra, 1995).

Examples of autoimmune antigens include IASβ chain (Topham et al., Proc. Natl. Acad. Sci., USA, 91:8005-8009; 1994). Vaccination of mice with an 18 amino acid peptide from IASβ chain has been demonstrated to provide protection and treatment to mice with experimental autoimmune encephalomyelitis (Topham et al., supra, 1994).

In addition, rdsRNA segments can be constructed that encode an adjuvant, and can be used to increase host immune responses to immunogens. The particular adjuvant encoded by the rdsRNA is not critical to the present invention and may, for example, be the A subunit of cholera toxin (i.e. CtxA; GenBank Accession No. X00171, AF175708, D30053, D30052), or parts and/or mutant derivatives thereof (e.g. the A1 domain of the A subunit of Ctx (i.e. CtxA 1; GenBank Accession No. K02679), from any classical Vibrio cholerae (e.g. V. cholerae strain 395, ATCC No. 39541) or El Tor V. cholerae (e.g. V. cholerae strain 2125, ATCC No. 39050) strain. Alternatively, any bacterial toxin that is a member of the family of bacterial adenosine diphosphate-ribosylating exotoxins (Krueger and Barbier, Clin. Microbiol. Rev., 8:34; 1995), may be used in place of CtxA; for example, the A subunit of heat-labile toxin (referred to herein as EltA) of enterotoxigenic Escherichia coli (GenBank Accession No. M35581), pertussis toxin S1 subunit (e.g. ptxS1, GenBank Accession No. AJ007364, AJ007363, AJ006159, AJ006157, etc.); as a further alternative, the adjuvant may be one of the adenylate cyclase-hemolysins of Bordetella pertussis (ATCC No. 8467), Bordetella bronchiseptica (ATCC No. 7773) or Bordetella parapertussis (ATCC No. 15237), e.g. the cyaA genes of B. pertussis (GenBank Accession No. X14199), B. parapertussis (GenBank Accession No. AJ249835) or B. bronchiseptica (GenBank Accession No. Z37112) and so on.

In a further alternative, the instant invention provides dsRNA segments that encode a pro-apoptosis protein (herein referred to as “PAP”), and direct tumor antigens to cross-prime antigen presentation pathways to induce the development of effector CD4⁺ and CD8⁺ T-cell responses. The link between apoptosis and the presentation of antigens by dendritic cells (DC)s, termed cross-priming (Heath & Carbone, Nat Rev Immunol 1:126; (2001); Heath & Carbone. Annu Rev Immunol 19:47 (2001); Sheridan et al., Science 277:818 (1997); Gallucci et al., Nat Med 5:1249 (1999); Ridge et al., Nature 393:474 (1998); Ignatius et al., J Virol 74:11329 (2000); Albert et al., J Exp Med 188:1359 (1998); Albert et al., Nature 392:86 (1998); Winau et al., Cell Microbiol 6:599 (2004); Schaible et al., Nat Med 9:1039 (2003)), has been implicated as a key initiating factor in the afferent events that lead to the development of effector CD4⁺ and CD8⁺ T-cell responses and results in the control and/or clearance of tumor cells (Rovere et al., J Leukoc Biol 66:345 (1999); Berard et al., J Exp Med 192:1535 (2000); Hoffmann et al., Cancer Res 60:3542 (2000); Zhou et al., Immunother 25:289 (2002); Scheffer et al., Int J Cancer 103:205 (2003)). Therefore, RPC-induced apoptosis provides a mechanism for the delivery of antigens to DCs, thereby leading to the induction of T cells.

Accordingly, the present invention provides dsRNA segments capable of expressing a PAP, such as, but not limited to, the mature activated form of caspase-8⁺ (GenBank Accession No. NP033942; i.e. amino acids 99-480). A preferred embodiment provides dsRNA segments capable of expressing a PAP from a microbial source, such as, but not limited to, the proteolytic domain of NS3 (spans amino acids 1-190; SEQ ID NO:7; herein designated “NS3_{Pr}”) encoded by base pairs 6469-7039 of West Nile virus isolate Mex03 (GenBank Accession No. AY660002), the hepatitis C virus core protein (GenBank Accession No. AAX11912), the cytomegalovirus-encoded chemokine receptor (GenBank Accession No. AAQ24855), the human herpes virus chemokine receptor US28 (GenBank Accession No. AAN37944), the lyssavirus matrix protein (GenBank Accession No. AY540348), the IpaB protein of Shigella flexneri (GenBank Accession No. AAM89543), and the SipB protein of Salmonella enterica (GenBank Accession No. 2123407B).

The sequences encoding the PAP can be generated synthetically by a commercial source (e.g. Picoscript, Houston Tex.; DNA 2.0, Menlo Park, Calif.) and can be optimized for expression in the target cell by using the preferred codon bias of the genus. Intercellular transport of the PAP from cell to cell of the host cell can be accomplished by creating a fusion between an intercellular trafficking protein (herein referred to as “ITP”); e.g., the human herpes virus tegument protein, VP22 (GenBank Accession No. BAE87004), the human immunodeficiency virus tat protein (GenBank Accession No. AAF35362), or parts thereof (e.g. VP22 amino acids 81-195 or tat amino acids 40-65), and the PAP. Expression of the PAP can be enhanced by placing a spacer between the ITP and the PAP, such as a flexible spacer (e.g. Serine-Glycine-Glycine-Glycine-Glycine-Serine; SEQ ID NO:8), an inflexible linker (e.g. Serine-Proline-Proline-Proline-Proline-Proline-Proline-Serine; SEQ ID NO:9) or flexible linker with a furin degradation motif (e.g. Serine-Glycine-Glycine-Glycine-Glycine-Arginine-Threonine-Lysine-Arginine-Glycine-Glycine-Glycine-Glycine-Serine; SEQ ID NO:10).

Expression of ITP::Linker::PAP (wherein “::” denotes the novel genetic junction) is accomplished by functionally linking dsRNA encoding said genetic fusions to the 5′-TLS, such the 5′-UTR of HCV (e.g. GenBank Accession No. BD161057) and/or a Kozak sequence translation enhancer, and 3′-TLS, such as the polypyrimidine tract sequence (e.g. Koh et al., J. Biol. Chem. 278:20565; 2003). Synthetic DNA encoding recombinant genes, 5′-TLS::LP_{Ag85A}::ITP::Linker::PAP::3′-TLS can be purchased from commercial sources (e.g. Picoscript, Houston, Tex.; DNA 2.0, Menlo Park, Calif.) and are introduced into RPCs as described herein.

In addition, or alternatively, it is possible to construct rdsRNA segments encoding a cytokine, which are useful as adjuvants or for the production of the recombinant proteins as therapeutics. The particular cytokine encoded by the rdsRNA is not critical to the present invention and includes, but is not limited to, interleukin-4 (herein referred to as “IL-4”); Genbank Accession No. AF352783 (murine IL-4) or NM_{—}000589 (human IL-4), IL-5 (Genbank Accession No. NM_{—}010558 (murine IL-5) or NM_{—}000879 (human IL-5)), IL-6 (Genbank Accession No. M20572 (murine IL-6) or M29150 (human IL-6)), IL-10 (Genbank Accession No. NM 010548 (murine IL-10) or AF418271 (human IL-10)), Il-12-p40 (Genbank Accession No. NM_{—}008352 (murine IL-12 p40) or AY008847 (human IL-12 p40)), IL-12-p70 (Genbank Accession No. NM_{—}008351/NM_{—}008352 (murine IL-12 p35/40) or AF093065/AY008847 (human IL-12 p35/40)), TGFβ (Genbank Accession No. NM_{—}011577 (murine TGFβ1) or M60316 (human TGFβ1), and TNFα Genbank Accession No. X02611 (murine TNFα) or M26331 (human TNFα).

Recombinant DNA and RNA procedures for the introduction of functional expression cassettes to generate rdsRNA capable of expressing an immunoregulatory agent in target host cells or tissues are described herein.

As exemplary vaccine constructs to be encoded in eukaryotic expression cassettes, virus-like particles (herein referred to as “VLP”) can be constructed to induce or to produce protective immune responses against viral pathogens. Influenza VLP's have been shown to self assemble following plasmid expression of gene sequences encoding the hemaglutinin (HA), neuramimidase (NA), and the matrix proteins (M1 and M2) (Latham et al., J. Virol, 75:6154-6165; 2001). VLP's so constructed are further capable of membrane fusion and budding to further potentiate antibody-producing immune responses and protective immunity in animal models (Pushko et al., Vaccine 23:5751; 2005). HIV VLP's can be similarly assembled from minimal sequences encoding amino acids 146-231 of the capsid protein, a six amino acid myristylation sequence, the sequence encoding the P2 peptide, a GCN4 leucine zipper domain, and the gp160 envelope precursor (Accola et al., J. Virol, 74:5395-5402; 2000). The major protein, LI, of HPV has been shown to self-assemble into VLP's a variety of cell lines and produces humoral and cellular immunity, making the gene encoding the protein a candidate immunogen or vaccine (Shi et al., J. Virol., 75(21): 10139-10148; 2001).

Recombinant DNA and RNA procedures for the introduction of functional expression cassettes to generate rdsRNA capable of expressing a VLP in target host cells or tissues are described herein.

In another embodiment, RPCs are produced to express recombinant proteins useful as therapeutics and laboratory reagents. Examples of recombinant proteins include but are not limited to: calcitonin, CTLA-Ig fusion protein, glucagon, hyaluronidase, insulin, insulin-like Growth-Factor-1, interferon α₂ₐ, interferon α_{2b}, parathyroid hormone, somatropin, somatropin antagonist, p53, platelet-derived growth factor, urate oxidase, factor VIII, factor VIIa, arylsulfatase B, bone morphogenic protein-2, bone morphogenic protein-7, DNase, erythropoietin, factor IX, follicle stimulating hormone, β-Beta-Galactosidase, glucocerebrosidase, glucosidase, human Glucocerebrosidase, Glucosidase, Human cariogenic hormone, iduronidase, iduronate, iduronate-2-sulfatase, luteinizing hormone, tumor necrosis factor receptor-IgG Fc fusion protein, tissue plasminogen activator (tPA), thyroid stimulating hormone, etc.

Recombinant DNA and RNA procedures for the introduction of functional expression cassettes to generate rdsRNA capable of expressing recombinant proteins in target host cells or tissues are described herein.

In a further embodiment, RPCs can be produced that express monoclonal antibodies mAbs, such as but not limited to anti-CD11a mAb, anti-CD20 mAb, anti-CD52 mAb, anti-HER2 receptor mAb, anti-immunoglobulin E mAb, anti-TNF mAb, anti-interleukin-2 receptor mAb, anti-platelet mAb, anti-RSV mAb, anti-EGF-receptor mAb etc.

A suitable transgene, target gene and so on for cloning in an RPC of interest is a binding partner of an antigen or epitope, such as, an antibody or antigen-binding fragment thereof.

The term “antibody” is used in the broadest sense, and specifically covers monoclonal antibodies (including full length monoclonal antibodies), polyclonal antibodies, multispecific antibodies (e.g., bispecific antibodies), antibody fragments or synthetic polypeptides carrying one or more CDR or CDR-derived sequences so long as the polypeptides exhibit the desired biological activity. Antibodies (Abs) and immunoglobulins (Igs) are glycoproteins having the same structural characteristics. Generally, antibodies are considered Igs with a defined or recognized specificity. Thus, while antibodies exhibit binding specificity to a specific target, immunoglobulins include both antibodies and other antibody-like molecules which lack target specificity. The antibodies of the invention can be of any class (e.g., IgG, IgE, IgM, IgD, IgA and so on), or subclass (e.g., IgG₁, IgG₂, IgG_{2a, IgG}₃, IgG₄, IgA₁, IgA₂ and so on) (“type” and “class”, and “subtype” and “subclass”, are used interchangeably herein). Native or wildtype, that is, obtained from a non-artificially manipulated member of a population, antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at the other end. By “non-artificially manipulated” is meant not treated to contain or express a foreign antigen binding molecule. Wildtype can refer to the most prevalent allele or species found in a population or to the antibody obtained from a non-manipulated animal, as compared to an allele or polymorphism, or a variant or derivative obtained by a form of manipulation, such as mutagenesis, use of recombinant methods and so on to change an amino acid of the antigen-binding molecule.

The term “antibody fragment” refers to a portion of an intact or a full-length chain or an antibody, generally the target binding or variable region. Examples of antibody fragments include, but are not limited to, F_{ab}, F_{ab′}, F_{(ab′)2} and Fᵥ fragments. A “functional fragment” or “analog of an antibody” is one which can prevent or substantially reduce the ability of the receptor to bind to a ligand or to initiate signaling. As used herein, functional fragment generally is synonymous with, “antibody fragment” and with respect to antibodies, can refer to fragments, such as Fᵥ, F_{ab}, F_{(ab′)2} and so on which can prevent or substantially reduce the ability of the receptor to bind to a ligand or to initiate signaling. An “Fᵥ” fragment consists of a dimer of one heavy and one light chain variable domain in a non-covalent association (V_{H}-V_{L} dimer). In that configuration, the three CDRs of each variable domain interact to define a target binding site on the surface of the V_{H}-V_{L} dimer, as in an intact antibody. Collectively, the six CDRs confer target binding specificity on the intact antibody. However, even a single variable domain (or half of an Fᵥ comprising only three CDRs specific for a target) can have the ability to recognize and to bind target.

“Single-chain Fᵥ,” “sFᵥ” or “scAb” antibody fragments comprise the V_{H} and V_{L} domains of an antibody, wherein these domains are present in a single polypeptide chain. Generally, the Fᵥ polypeptide further comprises a polypeptide linker, often a flexible molecule, between the V_{H} and V_{L} domains, which enables the sFv to form the desired structure for target binding.

The term “diabodies” refers to antibody fragments with two antigen-binding sites, which fragments can comprise a heavy chain variable domain (V_{H}) connected to a light chain variable domain (V_{L}) in the same polypeptide chain. By using a linker that is too short to allow pairing between the two variable domains on the same chain, the diabody domains are forced to pair with the binding domains of another chain to create two antigen-binding sites.

The F_{ab} fragment contains the variable and constant domains of the light chain and the variable and first constant domain (C_{H1}) of the heavy chain. F_{ab} fragments differ from F_{ab} fragments by the addition of a few residues at the carboxyl terminus of the C_{H1} domain to include one or more cysteines from the antibody hinge region. F_{ab′}, fragments can be produced by cleavage of the disulfide bond at the hinge cysteines of the F_{(ab′)2} pepsin digestion product. Additional enzymatic and chemical treatments of antibodies can yield other functional fragments of interest.

The term “monoclonal antibody” as used herein refers to an antibody obtained from a population of substantially homogeneous antibodies, i.e., the individual antibodies comprising the population are identical except for possible naturally occurring mutations that may be present in minor amounts.

Monoclonal antibodies herein specifically include “chimeric” antibodies in which a portion of the heavy and/or light chain is identical with or homologous to corresponding sequences in antibodies derived from a particular species or belonging to a particular antibody class or subclass (type or subtype), with the remainder of the chain(s) identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc Natl Acad Sci USA 81:6851 (1984)). Thus, CDRs from one class of antibody can be grafted into the FR of an antibody of different class or subclass.

Monoclonal antibodies are highly specific, being directed against a single target site, epitope or determinant. Furthermore, in contrast to conventional (polyclonal) antibody preparations which typically include different antibodies directed against different determinants (epitopes) of an antigen, each monoclonal antibody is directed against a single determinant on the target. In addition to their specificity, monoclonal antibodies are advantageous being synthesized by a host cell, uncontaminated by other immunoglobulins, and provides for cloning the relevant gene and mRNA encoding the antibody of chains thereof. The modifier “monoclonal” indicates the character of the antibody as being obtained from a substantially homogeneous population of antibodies, and is not to be construed as requiring production of the antibody by any particular method. For example, the monoclonal antibodies for use with the present invention may be isolated from phage antibody libraries using well known techniques or can be purified from a polyclonal prep. The parent monoclonal antibodies to be used in accordance with the present invention may be made by the hybridoma method described by Kohler et al., Nature 256:495 (1975), or may be made by recombinant methods well known in the art.

“Humanized” forms of non-human (e.g., murine) antibodies are chimeric immunoglobulins, immunoglobulin chains or fragments thereof (such as Fᵥ, F_{ab}/F_{ab′}, F_{(ab′)2} or other target-binding subsequences of antibodies) which contain sequences derived from non-human immunoglobulin, as compared to a human antibody. In general, the humanized antibody will comprise substantially all of one, and typically two, variable domains, in which all or substantially all of the CDR regions correspond to those of a non-human immunoglobulin and all or substantially all of the FR regions are those of a human immunoglobulin template sequence. The humanized antibody may also comprise at least a portion of an immunoglobulin constant region (F_{c}), typically that of the human immunoglobulin template chosen. In general, the goal is to have an antibody molecule that is minimally immunogenic in a human. Thus, it is possible that one or more amino acids in one or more CDRs also can be changed to one that is less immunogenic to a human host, without substantially minimizing the specific binding function of the one or more CDRs. Alternatively, the FR can be non-human but those amino acids most immunogenic are replaced with ones less immunogenic. Nevertheless, CDR grafting, as discussed above, is not the only way to obtain a humanized antibody. For example, modifying just the CDR regions may be insufficient as it is not uncommon for framework residues to have a role in determining the three-dimensional structure of the CDR loops and the overall affinity of the antibody for its ligand. Hence, any means can be practiced so that the non-human parent antibody molecule is modified to be one that is less immunogenic to a human, and global sequence identity with a human antibody is not always a necessity. So, humanization also can be achieved, for example, by the mere substitution of just a few residues, particularly those which are exposed on the antibody molecule and not buried within the molecule, and hence, not readily accessible to the host immune system. Such a method is taught herein with respect to substituting “mobile” or “flexible” residues on the antibody molecule, the goal being to reduce or dampen the immunogenicity of the resultant molecule without comprising the specificity of the antibody for its epitope or determinant. See, for example, Studnicka et al., Prot Eng 7(6)805-814, 1994; Mol 1 mm 44:1986-1988, 2007; Sims et al., J Immunol 151:2296 (1993); Chothia et al., J Mol Biol 196:901 (1987); Carter et al., Proc Natl Acad Sci USA 89:4285 (1992); Presta et al., J Immunol 151:2623 (1993), WO 2006/042333 and U.S. Pat. No. 5,869,619.

Strategies and methods for resurfacing antibodies, and other methods for reducing immunogenicity of antibodies within a different host, are disclosed, for example, in U.S. Pat. No. 5,639,641. Briefly, in a preferred method, (1) position alignments of a pool of antibody heavy and light chain variable regions are generated to yield heavy and light chain variable region framework surface exposed positions, wherein the alignment positions for all variable regions are at least about 98% identical; (2) a set of heavy and light chain variable region framework surface exposed amino acid residues is defined for a non-human, such as a rodent antibody (or fragment thereof); (3) a set of heavy and light chain variable region framework surface exposed amino acid residues that is most closely identical to the set of rodent surface exposed amino acid residues is identified; and (4) the set of heavy and light chain variable region framework surface exposed amino acid residues defined in step (2) is substituted with the set of heavy and light chain variable region framework surface exposed amino acid residues identified in step (3), except for those amino acid residues that are within 5 Å of any atom of any residue of a CDR of the rodent antibody, to yield a humanized, such as a rodent antibody retaining binding specificity.

Antibodies can be humanized by a variety of other techniques including CDR grafting (EPO 0 239 400; WO 91/09967; and U.S. Pat. Nos. 5,530,101 and 5,585,089), veneering or resurfacing (EPO 0 592 106; EPO 0 519 596; Padlan, 1991, Molec Imm 28(4/5):489-498; Studnicka et al., 1994, Prot Eng 7(6):805-814; and Roguska et al., 1994, PNAS 91:969-973) and chain shuffling (U.S. Pat. No. 5,565,332). Human antibodies can be made by a variety of methods known in the art including, but not limited to, phage display methods, see U.S. Pat. Nos. 4,444,887, 4,716,111, 5,545,806 and 5,814,318; and WO 98/46645, WO 98/50433, WO 98/24893, WO 98/16654, WO 96/34096, WO 96/33735 and WO 91/10741, using transgenic animals, such as rodents, using chimeric cells and so on.

“Antibody homolog” or “homolog” refers to any molecule which specifically binds the antigen of interest. Thus, an antibody homolog includes native or recombinant antibody, whether modified or not, portions of antibodies that retain the biological properties of interest, such as an F_{ab} or Fᵥ molecule, a single chain antibody, a polypeptide carrying one or more CDR regions and so on. The amino acid sequence of the homolog need not be identical to that of the naturally occurring antibody but can be altered or modified to carry substitute amino acids, inserted amino acids, deleted amino acids, amino acids other than the twenty normally found in proteins and so on to obtain a polypeptide with enhanced or other beneficial properties.

Antibodies with homologous sequences are those antibodies with amino acid sequences that have sequence homology with the amino acid sequence of a parent antibody of the present invention. Preferably, homology is with the amino acid sequence of the variable regions of an antibody of the present invention. “Sequence homology” as applied to an amino acid sequence herein is defined as a sequence with at least about 90%, 91%, 92%, 93%, 94% or more sequence homology, and more preferably at least about 95%, 96%, 97%, 98% or 99% sequence homology to another amino acid sequence, as determined, for example, by the FASTA search method in accordance with Pearson & Lipman, Proc Natl Acad Sci USA 85, 2444-2448 (1988).

A chimeric antibody is one with different portions of an antibody derived from different sources, such as different antibodies, different classes of antibody, different animal species, for example, an antibody having a variable region derived from a murine monoclonal antibody paired with a human immunoglobulin constant region and so on. Thus, a humanized antibody is a species of chimeric antibody. Methods for producing chimeric antibodies are known in the art, see, e.g., Morrison, 1985, Science 229:1202; Oi et al., 1986, BioTechniques 4:214; Gillies et al., 1989, J Immunol Methods 125:191-202; and U.S. Pat. Nos. 5,807,715, 4,816,567, and 4,816,397.

Artificial antibodies include scFv fragments, chimeric antibodies, diabodies, triabodies, tetrabodies and mru (see reviews by Winter & Milstein, 1991, Nature 349:293-299; and Hudson, 1999, Curr Opin Imm 11:548-557), each with antigen-binding or epitope-binding ability. In the single chain Fᵥ fragment (scFᵥ), the V_{H} and V_{L} domains of an antibody are linked by a flexible peptide. Typically, the linker is a peptide of about 15 amino acids. If the linker is much smaller, for example, 5 amino acids, diabodies are formed, which are bivalent scFv dimers. If the linker is reduced to less than three amino acid residues, trimeric and tetrameric structures are formed that are called triabodies and tetrabodies, respectively. The smallest binding unit of an antibody is a CDR, typically the CDR2 of the heavy chain which has sufficient specific recognition and binding capacity. Such a fragment is called a molecular recognition unit or mru. Several such mrus can be linked together with short linker peptides, therefore forming an artificial binding protein with higher avidity than a single mru.

Also included within the scope of the invention are functional equivalents of an antibody of interest. The term “functional equivalents” includes antibodies with homologous sequences, antibody homologs, chimeric antibodies, artificial antibodies and modified antibodies, for example, wherein each functional equivalent is defined by the ability to bind to the cognate antigen of the parent antibody. The skilled artisan will understand that there is an overlap in the group of molecules termed “antibody fragments” and the group termed “functional equivalents.” Methods of producing functional equivalents which retain cognate antigen binding ability are known to the person skilled in the art and are disclosed, for example, in WO 93/21319, EPO Ser. No. 239,400, WO 89/09622, EPO Ser. No. 338,745 and EPO Ser. No. 332,424.

The functional equivalents of the present application also include modified antibodies, e.g., antibodies modified by the covalent attachment of any type of molecule to the antibody. For example, modified antibodies include antibodies that have been modified, e.g., by glycosylation, acetylation, pegylation, deamidation, phosphorylation, amidation, derivatization by known protecting/blocking groups, proteolytic cleavage, linkage to a cellular ligand, linkage to a toxin or cytotoxic moiety or other protein etc. The covalent attachment need not yield an antibody that is immune from generating an anti-idiotypic response. The modifications may be achieved by known techniques, including, but not limited to, specific chemical cleavage, acetylation, formylation, metabolic synthesis etc. Additionally, the modified antibodies may contain one or more non-classical amino acids.

Many techniques are available to one of ordinary skill in the art which permit the optimization of binding affinity. Typically, the techniques involve substitution of various amino acid residues at the site of interest, followed by a screening analysis of binding affinity of the mutant polypeptide for the cognate antigen or epitope.

Once the antibody is identified and isolated, it is often useful to generate a variant antibody or mutant, or mutein, wherein one or more amino acid residues are altered, for example, in one or more of the hypervariable regions of the antibody. Alternatively, or in addition, one or more alterations (e.g., substitutions) of framework residues may be introduced in the antibody where these result in an improvement in the binding affinity of the antibody mutant for the cognate antigen. Examples of framework region residues that can be modified include those which non-covalently bind antigen directly (Amit et al., Science 233:747-753 (1986)); interact with/affect the conformation of a CDR (Chothia et al., J Mol Biol 196:901-917 (1987)); and/or participate in the V_{L}-V_{H} interface (EP 239 400). In certain embodiments, modification of one or more of such framework region residues results in an enhancement of the binding affinity of the antibody for the cognate antigen. For example, from about one to about five framework residues may be altered in this embodiment of the invention. Sometimes, this may be sufficient to yield an antibody mutant suitable for use in preclinical trials, even where none of the hypervariable region residues have been altered. Normally, however, the antibody mutant can comprise one or more hypervariable region alteration(s). The constant regions also can be altered to obtain desirable or more desirable effector properties.

The hypervariable region residues which are altered may be changed randomly, especially where the starting binding affinity of the parent antibody is such that randomly-produced antibody mutants can be readily screened for altered binding in an assay as taught herein.

Ordinarily, the antibody mutant with improved biological properties will have an amino acid sequence having at least 75% amino acid sequence identity or similarity with the amino acid sequence of either the heavy or light chain variable domain of the parent antibody, at least 80%, at least 85%, at least 90% and often at least 95% identity. Identity or similarity with respect to parent antibody sequence is defined herein as the percentage of amino acid residues in the candidate sequence that are identical (i.e., same residue) or similar (i.e., amino acid residue from the same group based on common side-chain properties, supra) with the parent antibody residues, after aligning the sequences and introducing gaps, if necessary, to achieve the maximum percent sequence identity.

Alternatively, antibody mutants can be generated by systematic mutation of the FR and CDR regions of the heavy and light chains, or the F_{c} region of the antibody of interest. Another procedure for generating antibody mutants involves the use of affinity maturation using phage display (Hawkins et al., J Mol Biol 254:889-896 (1992) and Lowman et al., Biochemistry 30(45):10832-10838 (1991)). Bacteriophage coat-protein fusions (Smith, Science 228:1315 (1985); Scott & Smith, Science 249:386 (1990); Cwirla et al. Proc Natl Acad Sci USA 8:309 (1990); Devlin et al. Science 249:404 (1990); Wells & Lowman, Curr Opin Struct Biol 2:597 (1992); and U.S. Pat. No. 5,223,409) are known to be useful for linking the phenotype of displayed proteins or peptides to the genotype of bacteriophage particles which encode them. The F_{ab} domains of antibodies have also been displayed on phage (McCafferty et al., Nature 348: 552 (1990); Barbas et al. Proc Natl Acad Sci USA 88:7978 (1991); and Garrard et al. Biotechnol 9:1373 (1991)).

Monovalent phage display consists of displaying a set of protein variants as fusions of a bacteriophage coat protein on phage particles (Bass et al., Proteins 8:309 (1990). Affinity maturation, or improvement of equilibrium binding affinities of various proteins, has previously been achieved through successive application of mutagenesis, monovalent phage display and functional analysis (Lowman & Wells, J Mol Biol 234:564 578 (1993); and U.S. Pat. No. 5,534,617), for example, by focusing on the CDR regions of antibodies (Barbas et al., Proc Natl Acad Sci USA 91:3809 (1994); and Yang et al., J Mol Biol 254:392 (1995)).

Libraries of many (for example, 10⁶ or more) protein variants, differing at defined positions in the sequence, can be constructed on bacteriophage particles, each of which contains DNA encoding the particular protein variant. After cycles of affinity purification, using an immobilized antigen, individual bacteriophage clones are isolated, and the amino acid sequence of the displayed protein is deduced from the DNA.

Following production of the antibody mutant, the biological activity of that molecule relative to the parent antibody can be determined as taught herein. As noted above, that may involve determining the binding affinity and/or other biological activities or physical properties of the antibody. In a preferred embodiment of the invention, a panel of antibody mutants is prepared and is screened for binding affinity for the antigen. One or more of the antibody mutants selected from the screen are optionally subjected to one or more further biological activity assays to confirm that the antibody mutant(s) have new or improved properties.

The antibody mutant(s) so selected may be subjected to further modifications, often depending on the intended use of the antibody. Such modifications may involve further alteration of the amino acid sequence, fusion to heterologous polypeptide(s) and/or covalent modifications. For example, a cysteine residue not involved in maintaining the proper conformation of the antibody mutant may be substituted, generally with serine, to improve the oxidative stability of the molecule and to prevent aberrant cross-linking. Conversely, a cysteine may be added to the antibody to improve stability (particularly where the antibody is an antibody fragment such as an Fᵥ fragment).

Another type of antibody mutant has an altered glycosylation pattern. That may be achieved by deleting one or more carbohydrate moieties found in the antibody and/or by adding one or more glycosylation sites that are not present in the antibody. Glycosylation of antibodies is typically either N-linked to Asn or O-linked to Ser or Thr. The tripeptide sequences, asparagine-X-serine and asparagine-X-threonine, where X is any amino acid except proline, are common recognition sequences for enzymatic attachment of a carbohydrate moiety to the asparagine side chain. N-acetylgalactosamine, galactose, fucose or xylose, for example, are bonded to a hydroxyamino acid, most commonly serine or threonine, although 5-hydroxyproline or 5-hydroxylysine also may be used. Addition or substitution of one or more serine or threonine residues to the sequence of the original antibody can enhance the likelihood of O-linked glycosylation.

It may be desirable to modify the antibody of the invention with respect to effector function, so as to enhance the effectiveness of the antibody. For example, cysteine residue(s) may be introduced in the F_{c} region, thereby allowing interchain disulfide bond formation in that region. The homodimeric antibody thus generated may have improved internalization capability and/or increased complement-mediated cell killing and antibody-dependent cellular cytotoxicity (ADCC), see Caron et al., J Exp Med 176:1191-1195 (1992) and Shopes, Immunol 148:2918-2922 (1993). Alternatively, an antibody can be engineered which has dual F_{c} regions and may thereby have enhanced complement lysis and ADCC capabilities, see Stevenson et al., Anti-Cancer Drug Design 3: 219 230 (1989).

Functional equivalents may be produced by interchanging different CDRs of different antibody chains within a framework or a composite FR derived from plural antibodies. Thus, for example, different classes of antibody are possible for a given set of CDRs by substitution of different heavy chains, for example, IgG₁₋₄, IgM, IgA₁₋₂ or IgD, to yield differing antibody types and isotypes. Similarly, artificial antibodies within the scope of the invention may be produced by embedding a given set of CDRs within an entirely synthetic framework.

The antibody fragments and functional equivalents of the present invention encompass those molecules with a detectable degree of specific binding to the cognate antigen. A detectable degree of binding includes all values in the range of at least 10-100%, preferably at least 50%, 60% or 70%, more preferably at least 75%, 80%, 85%, 90%, 95% or 99% of the binding ability of an antibody of interest. Also included are equivalents with an affinity greater than 100% that of an antibody of interest.

The CDRs generally are of importance for epitope recognition and antibody binding. However, changes may be made to residues that comprise the CDRs without interfering with the ability of the antibody to recognize and to bind the cognate epitope. For example, changes that do not impact epitope recognition, yet increase the binding affinity of the antibody for the epitope, may be made. Several studies have surveyed the effects of introducing one or more amino acid changes at various positions in the sequence of an antibody, based on the knowledge of the primary antibody sequence, on the properties thereof, such as binding and level of expression (Yang et al., 1995, J Mol Biol 254:392-403; Rader et al., 1998, Proc Natl Acad Sci USA 95:8910-8915; and Vaughan et al., 1998, Nature Biotechnology 16, 535-539).

Thus, equivalents of an antibody of interest can be generated by changing the sequences of the heavy and light chain genes in the CDR1, CDR2 or CDR3, or framework regions, using methods such as oligonucleotide-mediated site-directed mutagenesis, cassette mutagenesis, error prone PCR, DNA shuffling or mutator-strains of E. coli (Vaughan et al., 1998, Nat Biotech 16:535-539; and Adey et al., 1996, Chap. 16, pp. 277-291, in Phage Display of Peptides and Proteins, eds. Kay et al., Academic Press). The methods of changing the nucleic acid sequence of the primary antibody can result in antibodies with improved affinity (Gram et al., 1992, Proc Natl Acad Sci USA 89:3576-3580; Boder et al., 2000, Proc Natl Acad Sci USA 97:10701-10705; Davies & Riechmann, 1996, Immunotech 2:169-179; Thompson et al., 1996, J Mol Biol 256:77-88; Short et al., 2002, J Biol Chem 277:16365-16370; and Furukawa et al., 2001, J Biol Chem 276:27622-27628).

Repeated cycles of “polypeptide selection” can be used to select for higher and higher affinity binding by, for example, the selection of multiple amino acid changes which are selected by multiple selection of cycles. Following a first round of selection, involving a first region of selection of amino acids in the ligand or antibody polypeptide, additional rounds of selection in other regions or amino acids of the ligand are conducted. The cycles of selection are repeated until the desired affinity properties are achieved.

Recombinant DNA and RNA procedures for the introduction of functional expression cassettes to generate rdsRNA capable of expressing mAbs in target host cells or tissues are described herein.

Many techniques are available to one of ordinary skill in the art which permit manipulation of polypeptides. Typically, the techniques involve substitution of various amino acid residues at a site of interest, followed by a screening analysis of a function, for example, tropism or antigen binding.

One procedure for obtaining protein mutants, variants, derivatives, muteins and the like is “alanine scanning mutagenesis” (Cunningham & Wells, Science 244:1081-1085 (1989); and Cunningham & Wells, Proc Nat. Acad Sci USA 84:6434-6437 (1991)). One or more residues are replaced by alanine or polyalanine residue(s). Those residues demonstrating functional sensitivity to the substitutions then are refined by introducing further or other mutations at or for the sites of substitution. Thus, while the site for introducing an amino acid sequence variation is predetermined, the nature of the mutation per se need not be predetermined. Similar substitutions can be attempted with other amino acids, depending on the desired property of the scanned residues.

A more systematic method for identifying amino acid residues to modify comprises identifying residues involved in a function and those residues with little or no involvement with that function. An alanine scan of the involved residues is performed, with each ala mutant tested for altering the function of interest. In another embodiment, those residues with little or no involvement with the function of interest are selected to be modified. Modification can involve deletion of a residue or insertion of one or more residues adjacent to a residue of interest. However, normally the modification involves substitution of the residue by another amino acid. A conservative substitution can be a first substitution. If such a substitution results in a change from baseline of the function of interest, then another conservative substitution can be made to determine if more substantial changes are obtained.

Even more substantial modification can be accomplished by selecting an amino acid that differs more substantially in properties from that normally resident at a site. Thus, such a substitution can be made while maintaining: (a) the structure of the polypeptide backbone in the area of the substitution, for example, as a sheet or helical conformation; (b) the charge or hydrophobicity of the molecule at the target site, or (c) the bulk of the side chain.

For example, the naturally occurring amino acids can be divided into groups based on common side chain properties:

(1) hydrophobic: methionine (M or met), alanine (A or ala), valine (V or val), leucine (L or leu) and isoleucine (I or ile);

(2) neutral, hydrophilic: cysteine (C or cys), serine (S or ser), threonine (T or thr), asparagine (N or asn) and glutamine (Q or gln);

(3) acidic: aspartic acid (D or asp) and glutamic acid (E or glu);

(4) basic: histidine (H or his), lysine (K or lys) and arginine (R or arg);

(5) residues that influence chain orientation: glycine (G or gly) and proline (P or pro), and

(6) aromatic: tryptophan (W or trp), tyrosine (Y or tyr) and phenylalanine (F or phe).

Non-conservative substitutions can entail exchanging an amino acid with an amino acid from another group. Conservative substitutions can entail exchange of one amino acid for another within a group.

Preferred amino acid substitutions can include those which: (1) reduce susceptibility to proteolysis, (2) reduce susceptibility to oxidation, (3) alter immune system stimulating activity and/or (4) confer or modify other physico-chemical or functional properties of such analogs, such as enhancing serum half-life, toxin deactivation, antigen binding and so on. Analogs can include various muteins of a sequence other than the naturally occurring peptide sequence. For example, single or multiple amino acid substitutions (preferably conservative amino acid substitutions) may be made in the naturally-occurring sequence (for example, in the portion of the polypeptide outside the functional domain(s)). A conservative amino acid substitution generally should not substantially change the structural characteristics of the parent sequence (e.g., a replacement amino acid should not tend to break a helix that occurs in the parent sequence, or disrupt other types of secondary structure that characterizes the parent sequence) unless of a change in the bulk or conformation of the R group or side chain (Proteins, Structures and Molecular Principles (Creighton, ed., W. H. Freeman and Company, New York (1984); Introduction to Protein Structure, Branden & Tooze, eds., Garland Publishing, New York, N.Y. (1991)); and Thornton et al. Nature 354:105 (1991)).

Ordinarily, the protein, variant, mutant and so on with altered properties will have an amino acid sequence having at least 75% amino acid sequence identity or similarity with the amino acid sequence of the parent molecule, at least 80%, at least 85%, at least 90% and often at least 95% identity. Identity or similarity with respect to parent amino acid sequence is defined herein as the percentage of amino acid residues in the candidate sequence that are identical (i.e., same residue) or similar (i.e., amino acid residue from the same group based on common side-chain properties, supra) with the parent molecule residues, after aligning the sequences and introducing gaps, if necessary, to achieve the maximum percent sequence identity.

When suitable changes to the expressed polypeptide are identified, then suitable changes can be made to the nucleic acid encoding same so that a vector and expression system described herein can be used to produce quantities of that mutein, variant, derivative, mutant and so on. Hence, for example, once an antigen or antigen-binding molecule of interest is identified, if not already cloned, the nucleic acid encoding same is isolated practicing methods known in the art, and that nucleic acid encoding that antigen or antigen-binding molecule is engineered to be carried by and expressed by an RPC of interest as taught herein.

In another embodiment, rdsRP, rdsRN and RPCs are produced to silence or regulate host genes using trait modifying nucleic acids, which can be RNA's, such as, small inhibitory RNA, antisense RNA, microRNA, ribozymes and so on. Examples of small inhibitory RNA sequences include, but are not limited to: mammalian target of rapamycin siRNA; conserved regions in HIV-1 gag, pol, int and vpu genes (Chang et al., Gene Ther. 12:1133-1144 (2005)); epidermal growth factor receptor in glioma cells (Vollmann et al.; Int J. Oncol. 28:1531-1542 (2006)); surface antigen region of Hepatitis B virus (Giladi et al., Mol. Ther. 8:769-776 (2003)); and NS3 and NSSB regions of Hepatitis C virus (Takigawa et al., Microbiol Immunol. 48:591-598 (2004)).

In the case of expressing such RNA molecules of interest, the replication proficient capsids of interest are well suited as the RNA of interest is not expressed but instead is excised or removed from the vector sequence. Thus, high titer capsid preparations are needed to ensure adequate copies of RNA are obtained. The target RNA can be removed by directed excision of the RNA, for example, using particular endonucleases, and so on, practicing materials and methods known in the art.

Examples of antisense RNA sequences include, but are not limited to: human papilloma virus E6 and E7 genes; human Jun N-terminal kinase 1 (Betigeri et al., Mol. Pharm. 3:424-430 (2006)); human C-Met, a receptor tyrosine kinase, (Chu et al., Surg Neurol. 65:533-538 (2006)); and HIV-1 gag (Ramezani et al., Front Biosci. 11:2940-2948 (2006)).

Examples of ribozyme sequences include, but are not limited to: human CPEB3 gene (Salehi-Ashtiani et al., Science 313:1788-1792 (2006)); hepatitis delta virus (Been, Curr Top Microbiol Immunol. 307:47-65 (2006)); glmS genes of many Gram-positive bacteria; the universal ribozyme, ribonuclease P (Torres-Larios et al., Curr Opin Struct Biol. 16:327-335 (2006)); Neurospora Varkud satellite (VS) ribozyme (Hoffmann et al., Proc Natl Acad Sci USA 100:7003-7008 (2003)); multimeric hammerhead ribozymes against various HIV-1 genes (Ramezani et al., supra); and ribozymes targeting Hepatitis C virus 5′-prime and 3′-prime untranslated regions (Jarczak et al., FEBS J. 272:5910-5922 (2005)).

Said RPCs that express small inhibitory RNA, antisense RNA or ribozymes express and silence or regulate host genes are useful as immunosuppressants, immunoregulatory agents, and anticancer, viral and gene therapeutics.

Elements that Enhance Expression of the Gene of Interest

Secretion of proteins in mammalian cells can be enhanced by including a signal recognition particle (SRP), which constitutes a critical component of the biosynthetic pathway of most secretory proteins in mammalian cells that is required for elongation arrest (e.g. Strub et al., Mol. Cell. Biol. 11:3949-3959; 1991) and efficient targeting to the endoplasmic reticulum.

The order and magnitude in which segments are expressed by the dsRNA phage family is regulated to ensure stoichiometric expression of structural and non-structural proteins. Since that rate of viable phage production is proportional to procapsid levels and discontinued by the lysis proteins, the expression of proteins on segment-L is more efficient than those on segment-M and segment-S, which encode the late genes involved in phage envelope synthesis and host cell lysis. Accordingly, enhanced expression of the expression cassette is accomplished by constructing RPCs that harbor a recombinant segment-L, which in turn contains an expression cassette.

For example, rdsRNA segment-L for expression in mammalian cells can be comprised of the following elements:

1) the segment-L pac sequence containing the modified replication proficient terminal (i.e. 5′-GGAAAAAAAG-3′) (SEQ ID NO:11);

2) a 5′-TLS, such the 5′-UTR of HCV (e.g. GenBank Accession No. BD161057);

3) a Kozak sequence translation enhancer;

4) RE sites (e.g. MscI) located 3′ to the Kozak sequence so that MscI (a blunt-end RE) provides a translational start codon (i.e. AUG) which is functionally linked to the Kozak sequence;

5) a gene of interest, such as a gene encoding a recombinant protein, vaccine antigen or a therapeutic agent;

6) a 3′-TLS, such as the polypyrimidine tract sequence (e.g. Koh et al., J. Biol. Chem. 278:20565; 2003);

7) the segment-L 3′ RNA-dependent RNA polymerase recognition sequence (e.g. Hoogstraten, Virol., 272:218; 2000); and

8) a modified 3′ end containing a replication proficient sequence (e.g. 5′ CUCUCUUCCC-3′) (SEQ ID NO:4).

### Bacterial Host Strains

The bacterial strain in which the RPCs are produced in the present invention is not critical thereto and include, but are not limited to: Campylobacter spp, Neisseria spp., Haemophilus spp, Aeromonas spp, Francisella spp, Yersinia spp, Klebsiella spp, Bordetella spp, Legionella spp, Corynebacterium spp, Citrobacter spp, Chlamydia spp, Brucella spp, Pseudomonas spp, Helicobacter spp, or Vibrio spp.

The particular Campylobacter strain employed is not critical to the present invention. Examples of Campylobacter strains that can be employed in the present invention include but are not limited to: C. jejuni (ATCC Nos. 43436, 43437, 43438), C. hyointestinalis (ATCC No. 35217), C. fetus (ATCC No. 19438) C. fecalis (ATCC No. 33709), C. doylei (ATCC No. 49349) and C. coli (ATCC Nos. 33559, 43133).

The particular Yersinia strain employed is not critical to the present invention. Examples of Yersinia strains which can be employed in the present invention include: Y. enterocolitica (ATCC No. 9610), Y. pestis (ATCC No. 19428), Y. enterocolitica Ye03-R2 (al-Hendy et al., Infect. Immun., 60:870; 1992) or Y. enterocolitica aroA (O'Gaora et al., Micro. Path., 9:105; 1990).

The particular Klebsiella strain employed is not critical to the present invention. An example of Klebsiella strains that can be employed in the present invention includes K. pneumoniae (ATCC No. 13884).

The particular Bordetella strain employed is not critical to the present invention. Examples of Bordetella strains which can be employed in the present invention include B. pertussis and B. bronchiseptica (ATCC No. 19395).

The particular Neisseria strain employed is not critical to the present invention. Examples of Neisseria strains that can be employed in the present invention include N. meningitidis (ATCC No. 13077) and N. gonorrhoeae, (ATCC No. 19424) as well as the N. gonorrhoeae MS11 aro mutant (Chamberlain et al., Micro. Path., 15:51-63; 1993).

The particular Aeromonas strain employed is not critical to the present invention. Examples of Aeromonas strains that can be employed in the present invention include A. salminocida (ATCC No. 33658), A. schuberii (ATCC No. 43700), A. hydrophila and, A. eucrenophila (ATCC No. 23309).

The particular Francisella strain employed is not critical to the present invention. An example of Francisella strains that can be employed in the present invention includes F. tularensis (ATCC No. 15482).

The particular Corynebacterium strain employed is not critical to the present invention. An example of Corynebacterium strains that can be employed in the present invention includes C. pseudotuberculosis (ATCC No. 19410).

The particular Citrobacter strain employed is not critical to the present invention. An example of Citrobacter strains that can be employed in the present invention includes C. freundii (ATCC No. 8090).

The particular Chlamydia strain employed is not critical to the present invention. An example of Chlamydia strains that can be employed in the present invention includes C. pneumoniae (ATCC No. VR1310).

The particular Haemophilus strain employed is not critical to the present invention. Examples of Haemophilus strains that can be employed in the present invention include H. influenzae (Lee et al., J. Biol. Chem. 270:27151; 1995) and, H. somnus (ATCC No. 43625).

The particular Brucella strain employed is not critical to the present invention. An example of Brucella strains that can be employed in the present invention includes B. abortus (ATCC No. 23448).

The particular Legionella strain employed is not critical to the present invention. Examples of Legionella strains that can be employed in the present invention include L. pneumophila (ATCC No. 33156), or a L. pneumophila mip mutant (Ott, FEMS Micro. Rev., 14:161; 1994).

The particular Pseudomonas strain employed is not critical to the present invention. An example of Pseudomonas strains that can be employed in the present invention includes P. aeruginosa (ATCC No. 23267).

The particular Helicobacter strain employed is not critical to the present invention. Examples of Helicobacter strains that can be employed in the present invention include H. pylori (ATCC No. 43504) or H. mustelae (ATCC No. 43772).

The particular Vibrio strain employed is not critical to the present invention. Examples of Vibrio strains that can be employed in the present invention include Vibrio cholerae (ATCC No. 14035), Vibrio cincinnatiensis (ATCC No. 35912), a V. cholerae RSI virulence mutant (Taylor et al., J. Infect. Dis., 170:1518-1523; 1994) and the V. cholerae ctxA, ace, zot, cep mutant (Waldor et al., Infect Dis., 170:278-283; 1994).

In a preferred embodiment, the bacterial strain in which RPCs are produced in the present invention includes members of the Enterobacteriaceae, including, but not limited to, Escherichia spp, Shigella spp, and Salmonella spp. Gram-positive and acid-fast packaging and vector strains could similarly be constructed from Listeria monocytogenes or Mycobacterium spp., respectively.

The particular Escherichia strain employed is not critical to the present invention. Examples of Escherichia strains which can be employed in the present invention include Escherichia coli strains DH5α, HB101, HS-4, 4608-58, 1184-68, 53638-C-17, 13-80, and 6-81 (see, e.g. Sambrook et al., supra; Sansonetti et al., Ann Microbiol. (Inst. Pasteur), 132A:351; 1982), enterotoxigenic E. coli (see, e.g. Evans et al., Infect. Immun, 12:656; 1975), enteropathogenic E. coli (see, e.g. Donnenberg et al., J. Infect. Dis., 169:831; 1994), enteroinvasive E. coli (see, e.g. Small et al., Infect Immun., 55:1674; 1987) and enterohemorrhagic E. coli (see, e.g. McKee and O'Brien, Infect. Immun, 63:2070; 1995).

The particular Salmonella strain employed is not critical to the present invention. Examples of Salmonella strains that can be employed in the present invention include S. typhi (see, e.g. ATCC No. 7251), S. typhimurium (see, e.g. ATCC No. 13311), S. galinarum (ATCC No. 9184), S. enteriditis (see, e.g. ATCC No. 4931), Salmonella typhimurium (see, e.g. ATCC No. 6994), S. typhi aroC, aroD double mutant (see, e.g. Hone et al., Vacc., 9:810-816; 1991) and S. typhimurium aroA mutant (see, e.g. Mastroeni et al., Micro. Pathol., 13:477-491; 1992).

The particular Shigella strain employed is not critical to the present invention. Examples of Shigella strains that can be employed in the present invention include Shigella flexneri (see, e.g. ATCC No. 29903), S. flexneri CVD1203 (see, e.g. Noriega et al., Infect. Immun. 62:5168; 1994), S. flexneri 15D (see, e.g. Sizemore et al., Science 270:299; 1995), S. sonnei (see, e.g. ATCC No. 29930), and S. dysenteriae (see, e.g. ATCC No. 13313).

The particular Mycobacterium strain employed is not critical to the present invention. Examples of Mycobacterium strains that can be employed in the present invention include M. tuberculosis CDC1551 strain (see, e.g. Griffith et al., Am. J. Respir. Crit. Care Med. August; 152(2):808; 1995), M. tuberculosis Beijing strain (van Soolingen et al., J Clin Micro 33(12):3234-3238, 1995) H37Rv strain (ATCC No. 25618), M. tuberculosis pantothenate auxotroph strain (Sambandamurthy, Nat. Med. 2002 8(10):1171; 2002), M. tuberculosis rpoV mutant strain (Collins et al., Proc Natl Acad Sci USA. 92(17):8036; 1995), M. tuberculosis leucine auxotroph strain (Hondalus et al., Infect. Immun. 68(5):2888; 2000), BCG Danish strain (ATCC No. 35733), BCG Japanese strain (ATCC No. 35737), BCG Chicago strain (ATCC No. 27289), BCG Copenhagen strain (ATCC No. 27290), BCG Pasteur strain (ATCC No. 35734), BCG Glaxo strain (ATCC No. 35741), BCG Connaught strain (ATCC No. 35745) and), BCG Montreal (ATCC No. 35746).

The particular Listeria monocytogenes strain employed is not critical to the present invention. Examples of Listeria monocytogenes strains which can be employed in the present invention include L. monocytogenes strain 104035 (e.g. Stevens et al., J Virol 78:8210-8218; 2004) and mutant L. monocytogenes strains, such as (i) actA plcB double mutant (Peters et al., FEMS Immunology and Medical Microbiology 35: 243-253; 2003); (Angelakopoulous et al., Infect. and Immun. 70: 3592-3601; 2002); and (ii) dal dat double mutant lacking alanine racemase and D-amino acid aminotransferase (Thompson et al., Infect. and Immun. 66: 3552; 1998).

### Selection Strategies

Selection alleles and strategies for selecting bacteria that harbor RPCs are well known in the art and are described elsewhere (U.S. Pat. No. 7,018,835; US Publ. No. 20060115493). The particular selection allele that is incorporated into the rdsRNA segment is not important to the present invention and can be any allele that creates a selectable phenotype in the host bacterial strain, such as, but not limited to: agh encoding kanamycin-resistance (GenBank Accession No. ABI21734), bla encoding ampicillin-resistance (GenBank Accession No. AAB08872), etc.

To streamline approval from regulatory agencies, such as the US Food and Drug Administration or European Medicines Agency for human products and the US Department of Agriculture for veterinary products, biological pharmaceutics must meet purity, safety and potency standards defined by the pertinent regulatory agency. To produce an RPC that meets those standards, the recombinant organisms should be maintained in culture media that is, for example, certified free of transmissible spongiform encephalopathies (herein referred to as “TSE”). Synthetic DNA techniques are well known in the art and such DNA's can be obtained practicing known methods or purchased from commercial sources, such as DNA 2.0 Inc. (Menlo Park, Calif.), Blue Heron Biotechnology (Bothell, Wash.), Geneart Inc. (Toronto, Ont, Canada), and Genscript Inc. (Piscataway, N.J.). To synthesize expression cassettes, a series of short sequences 100-200 base pairs in length can be generated and ligated together to form the full-length sequence using procedures well know in the art. The synthetic DNA can be produced using an Applied Biosystems International ABI™ 3900 High-Throughput DNA Synthesizer (Foster City, Calif.) and procedures provided by the manufacturer. Plasmids harboring a sequence of interest are introduced into a host cell, for example, by electroporation and selection of lines carrying such plasmids is achieved, for example, by antibiotic selection, such as hyg, encoding hygromycin resistance (GenBank accession No. AF025746; AF025747) and aph from Tn903, which confers kanamycin resistance (herein referred to as “Kan^{R}”; GenBank accession No. U75323).

Preferably, plasmids harboring a sequence of interest carry a non-antibiotic selection marker since it is not always ideal to use antibiotic resistance markers for selection and maintenance of plasmids that are designed for use in humans and veterinary pharmaceutics. In a preferred embodiment, therefore, the present invention provides a novel selection strategy in which, for example, a catabolic enzyme is utilized as a selection marker by enabling the growth of host cells in medium containing a substrate of said catabolic enzyme as a carbon source. An example of such a catabolic enzyme includes, but is not restricted to, lacYZ encoding lactose uptake and β-galactosidase (Genbank accession Nos. J01636, J01637, K01483, or K01793). Other selection markers that provide a metabolic advantage in defined media include, but are not restricted to, galTK (GenBank Accession No. X02306) for galactose utilization, sacPA (GenBank Accession No. J03006) for sucrose utilization, trePAR (GenBank Accession No. Z54245) for trehalose utilization, xylAB (GenBank Accession Nos. CAB13644 and AAB41094) for xylose utilization, etc. Alternatively, the selection can involve the use of antisense mRNA to inhibit a toxic allele, such as the sacB allele (GenBank Accession No. NP_{—}391325), which renders cells sensitive to sucrose.

Alternatively, a suicide plasmid harboring the transgene of interest can be introduced into a host cell, for example, by electroporation and selection of lines carrying such plasmids can be achieved by, for example, antibiotic selection, such as, incorporating hyg which encodes the hygromycin resistance trait (GenBank accession No. AF025746; or AF025747) and Kan^{R} (GenBank accession no. U75323). The suicide plasmid can carry a sequence that is identical to a genomic homolog. The sequence allows recombination between the suicide plasmid and the genome resulting in integration of the suicide plasmid into the host cell genome. Methods for allelic exchange are described herein and as known in the art.

Selective medium containing the metabolite as a carbon source can be a modified Sauton's medium (herein defined as “MSM”) containing 0.5 g KH₂PO₄ (Sigma Cat. No. P9666), 0.5 g MgSO₄.7H2O (Sigma Cat. No. M5921-500G), 0.1 ml of 1% (w/v) ZnSO₄ (Sigma Cat. No. 35392-1L) solution, 5 ml of a 5% (v/v) Triton WR1339 (Sigma Cat. No. T8761) solution, 2.0 g citric acid (Sigma Cat. No. 251275), 0.05 g ferric ammonium citrate (Sigma Cat. No. F5879), 4.0 g asparagine (Sigma Cat. No. A4159), and 0.6 ml oleic acid (Research Diagnostics Cat. No. 01257), and the substrate of interest at an appropriate concentration in place of glycerol (e.g. 5 g lactose (Sigma Cat. No. 17814) per liter of medium). Add water to 950 ml, and dissolve reagents. Check the pH and adjust to pH 7.4 with 1M NaOH. Sterilize by autoclaving at 121° C. for 15 min.

Thus, in some embodiments, the selection marker provides a metabolic advantage to the host bacterial strain, such as lacYZ (GenBank Accession No. NC_{—}000913) encoding lactose uptake and fermentation, which confers the ability to utilize lactose as a carbon source to bacterial strains that are naturally deficient in lactose fermentation, such as Salmonella enteriditis, Mycobacterium spp. and Shigella spp. Other selection markers that provide a metabolic advantage in defined media include, but are not restricted to, galTK (GenBank Accession No. X02306) for galactose utilization, sacPA (GenBank Accession No. J03006) for sucrose utilization, trePAR (GenBank Accession No. Z54245) for trehalose utilization, and xylAB (GenBank Accession No. CAB 13644 and AAB41094) for xylose utilization, etc. Alternatively, the selection can involve the use of antisense mRNA to inhibit a toxic allele, such as the sacB allele (GenBank Accession No. NP_{—}391325), which renders E. coli, Shigella and Salmonella strains sensitive to sucrose. In another preferred embodiment, rdsRP/rdsRN confer resistance to colicins by expressing a colicin immunity gene, such as, but not limited to, cbi (GenBank Accession No. M36645), which confers resistance to colicin B; cdi (GenBank Accession No. X14941), which confers resistance to colicin D; cni (GenBank Accession No. X06933), which confers resistance to colicin N; imm (GenBank Accession No. J01566), which confers resistance to colicin E1; etc.

### Production of RPCs

Methods for the launching and production of the improved rdsRP and rdsRN as well as the RPCs are described, in part, in US Publ. No. 20060115493, herein incorporated by reference in entirety. In a preferred embodiment, RPCs are produced in bacterial strains by introducing RNA encoding all of the information necessary to produce the rdsRNA segments following uptake into the procapsid. The recombinant RNA segments may be encoded on plasmids, which may be co-introduced into a packaging strain. The genes encoding segment-L and hence the procapsids, may be present in a packaging strain on a plasmid or can be integrated into the chromosome of the packaging strain. As a further option, the (+ve)-RNA encoding segment-L may be introduced into the packaging strain in concert with (+ve)-RNA encoding recombinant segments-S and -M. Once the procapsid incorporates the recombinant ssRNA's (herein referred to as ssRNA) of segment-S and segment-M, which must be of sufficient size and display the appropriate packaging sequences, to produce a signal for the uptake of segment-L mRNA. The latter is then incorporated and all packaged ssRNA is converted to dsRNA, resulting in the generation of RPCs. At this point, the RPC is capable of generating recombinant segments-S and -M mRNA and segment-L mRNA; the latter expresses the proteins that constitute the procapsid, which uptake incorporates the recombinant segment and segment-L mRNA, then converted to dsRNA, thereby generating additional RPCs.

In vitro synthesized recombinant segment mRNA is introduced into packaging strains by electroporation. An electroporation medium is generated, composed of i) an electrocompetent bacterial strain, at a density of about 10⁸-10¹¹ cfu/ml for packaging, launching and producing RPCs, comprising a) genomic DNA comprising at least one non-reverting selectable phenotypic mutation; b) nucleic acid sequences encoding genes necessary for procapsid production; and c) one or more procapsids comprising proteins with RNA packaging and RNA polymerase activity; and. ii) 1 ng-1 mg, preferably 1 mcg-100 mcg, more preferably 5 mcg-40 mcg RNA encoding at least a gene product that complements said at least one selectable phenotypic mutation and an RNA of interest functionally linked to a eukaryotic translation initiation sequence.

The RPC's of interest are obtained from the host bacterial cells as known in the art. There are several considerations that enhance obtaining large numbers of functional capsids. First, it is preferable that lipopolysaccharide (LPS) and other such toxins that may be carried by a bacterial host be rendered inactive. Next, gentle procedures that do not place excessive mechanical pressure on the bacteria and on the virus are preferred. For example, gentle methods of cell lysis can be used, such as use of chelating agents, such as EDTA, enzymes, such as lysozyme, alterations in environment, such as temperature changes or osmotic changes can be used. It is preferred that detergents not be used in the isolation procedure.

Sometimes, bacterial lysates and the early purified samples may contain procapsids. Because procapsids are not useful for expression, it can be beneficial to separate procapsids from capsids. Procapsids have utility nevertheless, as immunogen or decoy. One approach to separating procapsids from capsids is by affinity separation means. Thus, an antibody to p8 can be used to selective positively for capsids. The p8 immunogen can be the recombinant molecule or capsids. To ensure specificity, the antibody should not react with procapsids. Hence, a p8 antibody is obtained practicing methods known in the art. Preferably, the antibody is a monoclonal antibody. The mAb to p8 then can be immobilized on a solid phase and the bacterial lysate is passed over the solid phase capturing the capsids, which are removed from the solid phase. In an alternative embodiment, an antibody to p1, p2, p7 or combination thereof is obtained practicing methods known in the art. Thus, recombinant molecules or procapsid can be used as antigen. The resulting antibody should not react with capsids. As taught herein, one way to use such an antibody is an affinity separation means where the procapsid antibody is affixed to a solid phase for a negative selection for capsids, where procapsids are bound to the solid phase, and capsids remain in solution. The solid phase can be beads, which can be packed into a column, the luminal surface of a tube or tubing, a plate suitable for panning and so on as known in the art.

### Expressing Genes of Interest in Target Host Cells

Expression in Monera

Once recombinant capsids are generated in a bacterial strain it then is possible to express a recombinant protein in the carrier strain. Since the capsids possess RNA-dependent RNA polymerase activity, the RPCs will produce (+-ve)-strand messenger RNA (mRNA) in the host bacterial strain. If the mRNA contains an RBS appropriate for expression in the host strain, the mRNA will be translated to produce a recombinant protein of interest.

Alternatively, the capsids can be produced in Escherichia coli and introduced into a target bacterial strain to produce a transient carrier state in which the gene of interest is expressed. That step can be accomplished by forming bacterial protoplasts, which are transfected by RPCs. On the other hand, the host strain in which the capsids are initially produced can be engineered to express the envelope structural and lytic functions of the dsRP. Such functions are encoded in segment-S and segment-M (e.g. from phi-6, phi-8 or phi-13), which can be incorporated into a plasmid or integrated into the chromosome using procedures described herein. To improve efficient production of infectious phage, the segment-S/M sequences can be placed under the control of a regulated promoter, such as, but not limited to, regulated promoter, such as the lacYZ promoter (GenBank Accession No. NC_{—}000913), araBAD (GenBank Accession No. K00953) or Tet-on promoters (GenBank Accession No. X00006). Another effective method of controlling the expression of segment-S and segment-M products is through the use of a resolvase system, such as, Cre/LoxP (GenBank Accession No. 1XO0_A and 1XO0_B), which can be used to remove a RNA polymerase terminator placed between the promoter and the segment of interest.

Once the RPCs are encapsulated in the phage lipid layer and the host production strain has been lysed, the encapsulated RPCs (herein referred to as “ERPCs”) are admixed with the target strain of interest. Since the lipid layer has tropism for Pseudomonas syringae LPS or pili, it is preferable that the target strain express those factors. Methods for making such recombinant strains are well know in the art (26-28).

The advantage of that approach is a well characterized seed of the host bacteria in which the gene of interest is expressed is produced and can be stored at −80° C. in a desired amount, such as, sufficient to initiate expression of the gene of interest in 1 to 10,000 liters (10⁹-10¹⁴ cfu), preferably 10 to 1000 liters (i.e. 10¹⁰-10¹³ cfu), thereby bypassing the need to develop master, working and production seeds of an RPC carrier strain. The results are substantial cost and time savings, and enabling and facilitating emergency preparedness and commercial exploitation.

Expression in Plantae

Once recombinant capsids are generated in a bacterial strain, introduction of the recombinant capsids into plant cells is possible, thereby enabling expressing a recombinant protein in the plant cells. Since the capsids possess RNA-dependent RNA polymerase activity, which operates independently of bacteria genomic genes, the rRPC/rdsRP/rdsRN will produce (+-ve)-strand messenger RNA (mRNA) in the host plant cell. If the mRNA contains a TLS appropriate for expression in the host strain, the mRNA will be translated to produce a recombinant protein of interest.

Accordingly, to express a recombinant protein of interest in a plant of interest, the recombinant capsids are launched in a bacterial host strain that is capable of invading the target plant cells. For example, to express a recombinant protein, Glycine max (L.) Kerr (soybean) leaves are used with Pseudomonas syringae pv. Glycinea as a delivery vehicle for the introduction of RPCs into the G. max cells. Since P. syringae is a natural host of phi-6, RPCs are launchable in that bacterial host. To limit damage by the invading bacterial vector, a murI mutation can be introduced in the vector bacterial strain, which results in arrested cell wall synthesis in the absence of an exogenous source of D-glutamate. To transfer the expression of the gene of interest beyond the initial site of delivery, the RPCs can be modified to enable the expression of fully functional viral RNA replicons (e.g. Marillonnet et al., Proc. Natl. Acad. Sci., 101:6852; 2004) that can assemble to form infectious non-replicating viral particles, which, in turn, disseminate systemically and enable expression of the passenger protein throughout the infected plant.

A similar strategy can be employed to enable expression of a trait modifying mRNA, siRNA, or anti-sense RNA. In that instance, the trait can be stabilized by using a retron sequence and reverse transcriptase (e.g. Shimamoto et al., J. Bacteriol., 180:2999; 1998) to convert an RNA sequence into DNA. Integration into the genome can be accomplished using a viral integrase (GenBank Accession No. U72726) and immortalization of the host cell can be accomplished using the vir genes of Agrobacterium tumorfacium (GenBank Accession No. J03320). That latter procedure can be performed using whole plants or single cell suspensions. Demonstration that the genes have been integrated into the host is established by performing PCR with genomic DNA.

Expression In Vitro

The RPCs are also compatible with in vitro translation systems. Although those systems have potential to revolutionize protein manufacturing, current systems require production of mRNA prior to in vitro translation. However, the use of RPCs will eliminate the need to produce mRNA. Initially, it is important to conduct a series of studies in small scale (1-50 ml) to achieve the following:

1) optimize RPC density;

2) optimize mRNA stability;

3) optimize translation initiation rate and;

4) expand longevity of the production run.

In vitro translation systems are prepared from bacterial, yeast, plant mammalian cell extracts are described ((Zubay, Annu Rev Genet. 7: 267-287 (1973); Pelham & Jackson, J Biochem 67: 247-256 (1976); Anderson et al., Meth Enzymol 101: 635-644 (1983)).

Expression in Mammalia

Similarly, RPCs are capable of expressing recombinant proteins and genetic trait-altering factors, such as siRNA and anti-sense RNA, in mammalian cells in vitro and in vivo. Given the auto-transfecting properties of the dsRNA phage capsids (Poranen et al., J Cell Biol, 147:671; 1999), RPCs can be introduced into mammalian cells following direct application (i.e. admixing RPCs with mammalian cells in vitro or following inoculation into the target site in the mammalian host (e.g. bovine, murine, human, etc) wherein expression is desired.

As above, the host strain in which the capsids are initially produced can be engineered to express the envelope structural and lytic functions of the dsRP. Such functions are encoded in segment-S and segment-M (e.g. from phi-6, phi-8 or phi-13), which can be incorporated into a plasmid of integrated into a chromosome using procedures described herein. To improve efficient production of infectious phage, the segment-S/M sequences can be placed under the control of a regulated promoter, such as, but not limited to, lacYZ (GenBank Accession No. NC_{—}000913), araBAD (GenBank Accession No. K00953) or tet promoters (GenBank Accession No. X00006). Another effective method of controlling the expression of segment-S and segment-M products is through the use of a resolvase system such as Cre/LoxP (GenBank Accession No. 1XO0_A and 1XO0_B), which can be used to remove an RNA polymerase terminator placed between the promoter and the segment of interest.

Once the RPCs are encapsulated in the phage lipid layer and the host production strain has been lysed, the encapsulated RPC (herein referred to as “eRPC”) are purified, for example, by size exclusion chromatography and introduced into the target mammalian cells or tissues. The wild-type lipid layer has tropism for Pseudomonas syringae LPS or pili; however, the tropism can be altered by modifying the receptor-binding domain (herein referred to as “RBD”) of gene 3 (Mindich, Microbiol. Mol. Biol. Rev., 63:149; 1999), so as to enable cell-specific or tissue-specific targeting. The particular replacement of the RBD is not important to the present invention and can include the RBD of adenylate cyclase of Bordetella pertussis (GenBank Accession No. CAA01202), which targets CD11b on dendritic cells; the B subunit of cholera toxin (GenBank Accession No. CAA53976), which targets cells expressing GM1 ganglioside; the mannose binding lectin of Narcissus pseudonarcissus (herein referred to as “NPL”), which binds to cells expressing a terminal mannose on surface glycoproteins, etc.

A similar strategy can be employed to enable expression of a trait modifying mRNA, siRNA, or anti-sense RNA in target mammalian cells. When pertinent, the trait can be stabilized by using a retron sequence and reverse transcriptase (e.g. Shimamoto et al., J. Bacteriol., 180:2999; 1998) to convert an RNA sequence into DNA. Integration into the genome can be accomplished using a viral integrase (GenBank Accession No. U72726). That procedure can be performed in vivo or in vitro using single cell suspensions. Demonstration that the genes have been integrated into the host can be established, for example, by performing PCR with genomic DNA.

A polypeptide expressed from the cargo gene or transgene can be obtained and purified practicing materials and methods known in the art. Thus, for example, affinity chromatography, liquid chromatography, centrifugation, precipitation and other separation means can be used to purify a polypeptide of interest. An RNA of interest can be obtained by removing the virus dsRNA from the capsid practicing methods known in the art and excising the RNA of interest from the vector practicing materials and methods known in the art.

### Uses

The specific method used to formulate the novel RPC and products therefrom formulations described herein is not critical to the present invention and can be selected from a physiological buffer (Felgner et al., U.S. Pat. No. 5,589,466); aluminum phosphate or aluminum hydroxyphosphate (e.g. Ulmer et al., Vaccine, 18:18 (2000)), monophosphoryl-lipid A (also referred to as MPL or MPLA; Schneerson et al., J. Immunol., 147:2136-2140 (1991); e.g. Sasaki et al., Inf. Immunol., 65:3520-3528 (1997); Lodmell et al., Vaccine, 18:1059-1066 (2000)), QS-21 saponin (e.g. Sasaki et al., J. Virol., 72:4931 (1998); dexamethasone (e.g. Malone et al., J. Biol. Chem. 269:29903 (1994)); CpG DNA sequences (Davis et al., J. Immunol., 15:870 (1998)); interferon-α (Mohanty et al., J. Chemother. 14(2):194-197, (2002)), or lipopolysaccharide (LPS) antagonist (Hone et al., J. Human Virol., 1: 251-256 (1998)).

The formulation herein also may contain more than one active compound as necessary for the particular indication being treated, preferably those with complementary activities that do not adversely impact each other. For example, it may be desirable to further provide an immunogen with an adjuvant. Such molecules suitably are present in combination in amounts that are effective for the purpose intended.

Thus, a first capsid can be admixed with a second capsid carrying different transgenes, or a recombinant product of an RPC can be used with a second component, such as a foreign antigen, a small molecule or a therapeutic moiety conjugated to or mixed with same, administered as a conjugate, separately in combination, mixed prior to use and so on, as a therapeutic (e.g. Levine et al., Eds., New Generation Vaccines, 2^{nd} edition. Marcel Dekker, Inc., New York, N.Y. (1997)). The amount of RPC or expressed product is not critical to the present invention but is typically an amount sufficient to obtain the desired response in the target host.

The term “small molecule” as well as the “therapeutic molecule” and analogous terms include, but are not limited to, peptides, peptidomimetics, amino acids, amino acid analogues, polynucleotides, polynucleotide analogues, carbohydrates, lipids, nucleotides, nucleotide analogues, organic or inorganic compounds (i.e., including heterorganic and/organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, combinations thereof and other pharmaceutically acceptable forms of such compounds which elicit or stimulate a pharmacologic response or are pharmacologically active.

Thus, in the case of a communicable disease, an RPC or expressed product thereof of the invention may be administered alone or in combination with other types of treatment, including conventional agents, such as, antibiotics or antivirals.

In addition, the RPC or product thereof of the instant invention may be conjugated to various effector molecules such as heterologous polypeptides, drugs, radionucleotides or toxins, see, e.g., WO 92/08495; WO 91/14438; WO 89/12624; U.S. Pat. No. 5,314,995; and EPO 396,387. An RPC or product thereof may be conjugated to a therapeutic moiety, such as, a cytotoxin (e.g., a cytostatic or cytocidal agent), a therapeutic agent, an antibiotic, an antiviral or a radioactive metal ion (e.g., a emitters such as, for example, ²¹³Bi). A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicine, doxorubicin, daunorubicin, dihydroxy anthracindione, mitoxantrone, mithramycin, actinomycin D, 1-dehydrotestosterone, glucocorticoids, procaine, tetracaine, lidocaine, propranolol and puromycin and analogs or homologues thereof. Therapeutic agents include, but are not limited to, antimetabolites (e.g., methotrexate, 6-mercaptopurine, 6-thioguanine, cytarabine, 5-fluorouracil and decarbazine), alkylating agents (e.g., mechlorethamine, chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin, daunomycin and doxorubicin), antibiotics (e.g., dactinomycin, actinomycin, bleomycin, mithramycin and anthramycin (AMC)), and anti-mitotic agents (e.g., vincristine and vinblastine).

The present invention also is directed to therapies which involve administering an RPC or product thereof of the invention to an animal, a mammal, or a human, for treating, for example, TB, HIV, an infectious disease, such as, malaria, a cancer, such as, bladder cancer, ocular squamous cell carcinoma, vulval papilloma and so on, or other disorder when used for producing or as an adjuvant. The animal or subject may be a mammal in need of a particular treatment, such as a mammal having been diagnosed with a particular disorder, e.g., TB, before or after anthrax exposure or bladder cancer. For example, by administering a therapeutically acceptable dose of an RPC of the instant invention, or a cocktail of a plurality of the different RPC's or equivalents thereof, in combination with another therapeutic product or foreign antigen, disease symptoms may be ameliorated or prevented in the treated mammal, particularly humans.

Therapeutic compounds of the invention alleviate at least one symptom associated with a disease, disorder, or condition amenable for treatment with an RPC or product thereof of interest. The products of the invention may be provided in pharmaceutically acceptable compositions as known in the art or as described herein. The term “physiologically acceptable,” “pharmacologically acceptable” and so on mean approved by a regulatory agency of the Federal or a state government or listed in the U.S. Pharmacopeia or other generally recognized pharmacopeia for use in animals and more particularly in humans. A product of interest also can be admixed with a compound or composition found by a governing regulatory body as one which is generally regarded as safe (GRAF). Hence, the instant invention also relates to generally regarded as safe carriers, excipients and diluents, which, for example, can be a foodstuff, whether solid or liquid, edible by an animal or human.

As used herein, the phrase “low to undetectable levels of aggregation” refers to liquid samples containing no more than 5%, no more than 4%, no more than 3%, no more than 2%, no more than 1% and often no more than 0.5% aggregation, by weight protein, as measured by, for example, high performance size exclusion chromatography (HPSEC). Hence, in the case of a vaccine or an antibody, a liquid sample of same can be with low to undetectable levels of aggregated protein.

As used herein, the term “low to undetectable levels of fragmentation” refers to samples containing equal to or more than 80%, 85%, 90%, 95%, 98% or 99%, of the total protein, for example, in a single peak, as determined by HPSEC, or in two (2) peaks (heavy chain and light chain) by, for example, reduced capillary gel electrophoresis (rCGE) and containing no other single peaks having more than 5%, more than 4%, more than 3%, more than 2%, more than 1% or more than 0.5% of the total protein, each. The rCGE as used herein refers to capillary gel electrophoresis under reducing conditions sufficient to reduce disulfide bonds in an antibody or antibody-type or derived molecule. Hence, in the case of a vaccine or an antibody, a liquid sample of same can be with low to undetectable levels of protein fragments.

The antibody or variant, optionally can be formulated with one or more agents currently used to prevent or treat the disorder in question. The effective amount of such other agents depends on the amount of antibody present in the formulation, the type of disorder or treatment and other factors discussed above. Such are generally used in the same dosages and with administration routes as used hereinbefore or about from 1 to 99% of the heretofore employed dosages.

As used herein, the term “effective amount” refers to the amount of a therapy (e.g., a prophylactic or therapeutic agent), which is sufficient to reduce the severity and/or duration of a disease, ameliorate one or more symptoms thereof treatable by a product of interest, prevent the advancement of a disease or cause regression of a disease treatable with a product of interest, or which is sufficient to result in the prevention of the development, recurrence, onset, or progression of a disease or one or more symptoms thereof treatable with a product of interest, or enhance or improve the prophylactic and/or therapeutic effect(s) of another therapy useful for treating a disease. For example, a treatment of interest can reduce circulating toxin or pathogen, based on baseline or a normal level, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In another embodiment, an effective amount of a therapeutic or a prophylactic agent reduces the symptoms of a disease, such as influenza, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. Also used herein as an equivalent is the term, “therapeutically effective amount.” Also, a treatment of interest can increase survivability of the host, based on baseline or a normal level, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%.

The amount of therapeutic product which will be effective in the use or treatment of a particular disorder or condition will depend on the nature of the disorder or condition, and can be determined by standard clinical techniques. Where possible, a dose/response curve and the pharmaceutical compositions of the invention can be first derived in vitro. If a suitable animal model system is available, again a dose/response curve can be obtained and used to extrapolate a suitable human dose practicing methods known in the art. However, based on common knowledge of the art, a pharmaceutical composition effective in promoting a diminution of an inflammatory effect, for example, may provide a local therapeutic agent concentration of between about 5 and 20 ng/ml, and, preferably, between about 10 and 20 ng/ml. In an additional specific embodiment of the invention, a pharmaceutical composition effective in ameliorating the growth and survival of cells responsible for B cell-dependent autoimmune manifestations or graft rejection may provide a local therapeutic agent concentration of between about 10 ng/ml and about 100 ng/ml.

For example, in an embodiment, an aqueous solution of therapeutic polypeptide, such as an antibody or vaccine can be administered by subcutaneous injection. Each dose may range from about 0.5 mg to about 50 mg per kilogram of body weight, or more preferably, from about 3 mg to about 30 mg per kilogram body weight. The dosage can be ascertained empirically for the particular disease, patient population, mode of administration and so on, practicing pharmaceutic methods known in the art.

The dosing schedule for subcutaneous administration may vary from once a week to daily depending on a number of clinical factors, including the type of disease, severity of disease and the sensitivity of the subject to the therapeutic agent.

But the products of interest can be administered to a mammal in any acceptable manner. Methods of introduction include, but are not limited to, parenteral, subcutaneous, intraperitoneal, intrapulmonary, intranasal, epidural, inhalation and oral routes, and if desired for immunosuppressive treatment, intralesional administration. Parenteral infusions include intramuscular, intradermal, intravenous, intraarterial or intraperitoneal administration. The products or compositions may be administered by any convenient route, for example, by infusion or bolus injection, by absorption through epithelial or mucocutaneous linings (e.g., oral mucosa, rectal and intestinal mucosa etc.) and may be administered together with other biologically active agents. Administration can be systemic or local. In addition, it may be desirable to introduce the therapeutic products or compositions of the invention into the central nervous system by any suitable route, including intraventricular and intrathecal injection; intraventricular injection may be facilitated by an intraventricular catheter, for example, attached to a reservoir, such as an Ommaya reservoir. In addition, the product can be suitably administered by pulse infusion, particularly with declining doses of the products of interest. Preferably, the dosing is given by injection, preferably intravenous or subcutaneous injections, depending, in part, on whether the administration is brief or chronic.

Various other delivery systems are known and can be used to administer a product of the present invention, including, e.g., encapsulation in liposomes, microparticles or microcapsules (see Langer, Science 249:1527 (1990); Liposomes in the Therapy of Infectious Disease and Cancer, Lopez-Berestein et al., eds., (1989)).

The active ingredients may be entrapped in a microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences, 16th edition, A. Osal, Ed. (1980). The expressed product may be an antigen-binding molecule, the various forms being known in the art, which can be carried with a microcapsule as the payload of that package, or displayed at the surface thereof to yield a targeting means.

Pulmonary administration can also be employed, e.g., by use of an inhaler or nebulizer, and formulation with an aerosolizing agent. The composition of interest may also be administered into the lungs of a patient in the form of a dry powder composition, see e.g., U.S. Pat. No. 6,514,496.

It may be desirable to administer the therapeutic products or compositions of the invention locally to the area in need of treatment; that may be achieved by, for example, and not by way of limitation, local infusion, topical application, by injection, by means of a catheter, by means of a suppository or by means of an implant, said implant being of a porous, non-porous or gelatinous material, including hydrogels or membranes, such as sialastic membranes or fibers. Preferably, when administering a product of the invention, care is taken to use materials to which the RPC or product thereof does not absorb or adsorb.

In yet another embodiment, the products of interest can be delivered in a controlled release system. In one embodiment, a pump may be used (see Langer, Science 249:1527 (1990); Sefton, CRC Crit. Ref Biomed Eng 14:201 (1987); Buchwald et al., Surgery 88:507 (1980); and Saudek et al., N Engl J Med 321:574 (1989)). In another embodiment, polymeric materials can be used (see Medical Applications of Controlled Release, Langer et al., eds., CRC Press (1974); Controlled Drug Bioavailability, Drug Product Design and Performance, Smolen et al., eds., Wiley (1984); Ranger et al., J Macromol Sci Rev Macromol Chem 23:61 (1983); see also Levy et al., Science 228:190 (1985); During et al., Ann Neurol 25:351 (1989); and Howard et al., J Neurosurg 71:105 (1989)). In yet another embodiment, a controlled release system can be placed in proximity of the therapeutic target.

Therapeutic formulations of the product may be prepared for storage as lyophilized formulations or as aqueous solutions by mixing the product having the desired degree of purity with optional pharmaceutically acceptable carriers, diluents, excipients or stabilizers typically employed in the art, i.e., buffering agents, stabilizing agents, preservatives, isotonifiers, non-ionic detergents, antioxidants and other miscellaneous additives, see Remington's Pharmaceutical Sciences, 16th ed., Osol, ed. (1980). Such additives are generally nontoxic to the recipients at the dosages and concentrations employed, hence, the excipients, diluents, carriers and so on are pharmaceutically acceptable.

An “isolated” or “purified” RPC or product therefrom is substantially free of contaminating cells, lysate, proteins and so on from the cells and/or the medium from which the product of interest is obtained, or substantially free of chemical precursors or other chemicals in the medium used which contains components that are chemically synthesized. The language “substantially free of subcellular material” or “substantially free of non-RPC material” includes preparations of an RPC or a product thereof in which the product of interest is separated from other subcellular components of the cells, such as dead cells, and portions of cells, such as cell membranes, ghosts and the like, from which same is isolated or recombinantly produced. Thus, an RPC or product thereof that is substantially free of subcellular material or non-RPC material includes preparations of the cell or of the product thereof having less than about 30%, 20%, 25%, 20%, 10%, 5%, 2.5% or 1%, (by dry weight) of subcellular or non-RPC contaminants. The RPC or product thereof is separated or purified practicing methods known in the art.

As used herein, the terms “stability” and “stable” in the context of a liquid formulation comprising an RPC or product thereof, such as an antibody, refers to the resistance of the product in the formulation to thermal and chemical aggregation, degradation or fragmentation under given manufacture, preparation, transportation and storage conditions, such as, for one month, for two months, for three months, for four months, for five months, for six months or more. The “stable” formulations of the invention retain biological activity equal to or more than 80%, 85%, 90%, 95%, 98%, 99% or 99.5% under given manufacture, preparation, transportation and storage conditions. The stability of said RPC or product thereof preparation can be assessed by degrees of aggregation, degradation or fragmentation by methods known to those skilled in the art, including, but not limited to, physical observation, such as, with a microscope, particle size and count determination and so on, compared to a reference.

The instant invention encompasses formulations, such as, liquid formulations having stability at temperatures found in a commercial refrigerator and freezer found in the office of a physician or laboratory, such as from about −20° C. to about 5° C., said stability assessed, for example, by microscopic analysis, for storage purposes, such as for about 60 days, for about 120 days, for about 180 days, for about a year, for about 2 years or more. The liquid formulations of the present invention also exhibit stability, as assessed, for example, by particle analysis, at room temperatures, for at least a few hours, such as one hour, two hours or about three hours prior to use.

The term, “carrier,” refers to a diluent, adjuvant, excipient or vehicle with which the therapeutic is administered. Such physiological carriers can be sterile liquids, such as water and oils, including those of petroleum, animal, vegetable or synthetic origin, such as peanut oil, soybean oil, mineral oil, sesame oil and the like. Water is a suitable carrier when the pharmaceutical composition is administered intravenously. Saline solutions and aqueous dextrose and glycerol solutions also can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

The compositions can take the form of solutions, suspensions, emulsion, tablets, pills, capsules, powders, sustained-release formulations, depots and the like. The composition can be formulated as a suppository, with traditional binders and carriers such as triglycerides. Oral formulations can include standard carriers, such as, pharmaceutical grades of mannitol, lactose, starch, magnesium stearate, sodium saccharine, cellulose, magnesium carbonate etc. Examples of suitable carriers are described in “Remington's Pharmaceutical Sciences,” Martin. Such compositions will contain an effective amount of the RPC or product thereof, or variant thereof, preferably in purified form, together with a suitable amount of carrier so as to provide the form for proper administration to the patient. As known in the art, the formulation will be constructed to suit the mode of administration.

Buffering agents help to maintain the pH in the range which approximates physiological conditions or maintains a product of interest in a biologically active configuration or conformation. Buffers are preferably present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid-monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used.

Preservatives may be added to retard microbial growth, and may be added in amounts ranging from 0.2%-1% (w/v). Suitable preservatives for use with the present invention include phenol, benzyl alcohol, m-cresol, octadecyldimethylbenzyl ammonium chloride, benzylconium halides (e.g., chloride, bromide and iodide), hexamethonium chloride, alkyl parabens, such as, methyl or propyl paraben, catechol, resorcinol, cyclohexanol and 3-pentanol.

Isotonicifiers are present to ensure physiological isotonicity of liquid compositions of the instant invention and include polyhydric sugar alcohols, preferably trihydric or higher sugar alcohols, such as glycerin, erythritol, arabitol, xylitol, sorbitol and mannitol. Polyhydric alcohols can be present in an amount of between about 0.1% to about 25%, by weight, preferably 1% to 5% taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols; amino acids, such as arginine, lysine, glycine, glutamine, asparagine, histidine, alanine, ornithine, L-leucine, 2-phenylalanine, glutamic acid, threonine etc.; organic sugars or sugar alcohols, such as lactose, trehalose, stachyose, arabitol, erythritol, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thiosulfate; low molecular weight polypeptides (i.e., <10 residues); proteins, such as human serum albumin, bovine serum albumin, gelatin or immunoglobulins; hydrophilic polymers, such as polyvinylpyrrolidone, saccharides, monosaccharides, such as xylose, mannose, fructose or glucose; disaccharides, such as lactose, maltose and sucrose; trisaccharides, such as raffinose; polysaccharides, such as, dextran and so on. Stabilizers can be present in the range from 0.1 to 10,000 w/w per part of bacterium or product thereof.

Additional miscellaneous excipients include bulking agents, (e.g., starch), chelating agents (e.g., EDTA), antioxidants (e.g., ascorbic acid, methionine or vitamin E) and cosolvents.

As used herein, the term “surfactant” refers to organic substances having amphipathic structures, namely, are composed of groups of opposing solubility tendencies, typically an oil-soluble hydrocarbon chain and a water-soluble ionic group. Surfactants can be classified, depending on the charge of the surface-active moiety, into anionic, cationic and nonionic surfactants. Surfactants often are used as wetting, emulsifying, solubilizing and dispersing agents for various pharmaceutical compositions and preparations of biological materials.

Non-ionic surfactants or detergents (also known as “wetting agents”) may be added to help solubilize the therapeutic agent, as well as to protect the therapeutic protein against agitation-induced aggregation, which also permits the formulation to be exposed to shear surface stresses without causing denaturation of the protein. Suitable non-ionic surfactants include polysorbates (20, 80 etc.), polyoxamers (184, 188 etc.), Pluronic® polyols and polyoxyethylene sorbitan monoethers (TWEEN-20®, TWEEN-80® etc.). Non-ionic surfactants may be present in a range of about 0.05 mg/ml to about 1.0 mg/ml, preferably about 0.07 mg/ml to about 0.2 mg/ml.

As used herein, the term, “inorganic salt,” refers to any compound, containing no carbon, that results from replacement of part or all of the acid hydrogen or an acid by a metal or a group acting like a metal, and often is used as a tonicity adjusting compound in pharmaceutical compositions and preparations of biological materials. The most common inorganic salts are NaCl, KCl, NaH₂PO₄ etc.

The present invention provides liquid formulations of an RPC or product thereof, having a pH ranging from about 5.0 to about 7.0, or about 5.5 to about 6.5, or about 5.8 to about 6.2, or about 6.0, or about 6.0 to about 7.5, or about 6.5 to about 7.0.

Examples of diluents include a phosphate buffered saline, buffer for buffering against gastric acid in the bladder, such as citrate buffer (pH 7.4) containing sucrose, bicarbonate buffer (pH 7.4) alone, or bicarbonate buffer (pH 7.4) containing ascorbic acid, lactose, or aspartame. Examples of carriers include proteins, e.g., as found in skim milk, sugars, e.g., sucrose, or polyvinylpyrrolidone. Typically these carriers would be used at a concentration of about 0.1-90% (w/v) but preferably at a range of 1-10% (w/v).

The formulations to be used for in vivo administration must be sterile. That can be accomplished, for example, by filtration through sterile filtration membranes. For example, the subcellular formulations of the present invention may be sterilized by filtration.

Sustained-release preparations may be prepared for use with the products of interest. Suitable examples of sustained-release preparations include semi-permeable matrices of solid hydrophobic polymers containing the bacterium, or functional portion or variant thereof, and/or foreign antigen, which matrices are in the form of shaped articles, e.g., films or matrices. Suitable examples of such sustained-release matrices include polyesters, hydrogels (for example, poly(2-hydroxyethylmethacrylate), poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers (such as injectable microspheres composed of lactic acid-glycolic acid copolymer) and poly-D-(+3-hydroxybutyric acid. While polymers such as ethylene-vinyl acetate and lactic acid-glycolic acid enable release of molecules for over 100 days, certain hydrogels release cells, proteins and products for and during shorter time periods. Rational strategies can be devised for stabilization depending on the mechanism involved.

The RPC or product thereof composition will be formulated, dosed and administered in a manner consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the RPC or product thereof to be administered will be governed by such considerations, and can be the minimum amount necessary to prevent, ameliorate or treat a disease, condition or disorder.

The amount of the RPC or product thereof of the present invention to be administered will vary depending on the species of the subject, as well as the disease or condition that is being treated. Generally, the dosage employed will be about 10³ to 10¹¹ capsids, preferably about 10⁵ to 10⁹ capsids.

Where necessary, the composition may also include a solubilizing agent and a local anesthetic such as lidocaine or other “caine” anesthetic to ease pain at the site of the injection.

Generally, the ingredients are supplied either separately or mixed together in unit dosage form, for example, as a dry lyophilized powder or water-free concentrate in a sealed container, such as an ampule or sachet indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided, for example, in a kit, so that the ingredients may be mixed prior to administration.

An article of manufacture containing materials useful for the treatment of the disorders described above is provided. The article of manufacture comprises a container and a label. Suitable containers include, for example, bottles, vials, syringes and test tubes. The containers may be formed from a variety of materials such as glass or plastic. The container holds a composition which is effective for preventing or treating a condition or disease and may have a sterile access port (for example, the container may be an intravenous solution bag or a vial having a stopper pierceable by a hypodermic injection needle). The label on or associated with the container indicates that the composition is used for treating the condition of choice. The article of manufacture may further comprise a second container comprising a pharmaceutically acceptable buffer, such as phosphate-buffered saline, Ringer's solution and dextrose solution. It may further include other materials desirable from a commercial and user standpoint, including buffers, diluents, filters, needles, syringes and package inserts with instructions for use.

### EXAMPLES

The following examples are provided for illustrative purposes only, and are in no way intended to limit the scope of the present invention, which is described fully hereinabove.

### Example 1

### Construction of Recombinant Segments for Replication Proficient rdsRN

Recombinant segments for use in generating replication proficient rdsRN were designed such that each segment carries a prokaryotic and eukaryotic cassette. The recombinant segments described in this example are capable of expressing reporter genes, such as luciferase and lacZ. Recombinant segment-S (herein referred to as “BI10”; SEQ ID 1) and recombinant segment M (herein referred to as “BI8”; SEQ ID 2) were generated by synthetic DNA procedures and inserted into plasmid pJ53 (DNA 2.0). BI10 encodes, starting at the 5′-end: SP6 promoter→phi-8 pac S→phi-8(gene 8)→lacZa→HCV 5′TLS→red fluorescent protein (HcRed1)→HCV 3′TLS→phi-8 segment S 3′UTR. BI8 encodes, starting at the 5′-end: SP6 promoter→phi-8 pacM→phi-8(gene 12)→bla (Amp^{R})→agh (Kan^{R})→HCV 5′TLS→firefly luciferase→HCV 3′TLS→phi-8 segment M 3′UTR.

### Example 2

### Functionality of Hepatitis C Virus-Derived TLS

US Patent Application no. 20060115493 describes the use of hepatitis C virus IRES immediately upstream of the gene of interest for control of eukaryotic translation. For example, recombinant segment M in US Patent Application no. 20060115493 consisted of the following: 5′-T7 promoter→phi-8 pacM→asd→HCV IRES→firefly luciferase→poly A sequence→phi-8 segment M 3′UTR.

In the present application, recombinant segments are configured such that eukaryotic translation is controlled by 5′ and 3′ TLS derived from HCV (see BI8 and BI10 in Example 1). Therefore, expression of firefly luciferase in recombinant segment M from US Patent Application No. 20060115493 was compared to BI16, which is BI8 with a T7 promoter. The comparison was performed using TNT™ coupled rabbit reticulocyte lysate system (Promega), a system that permits transcription and translation in the same tube. Each reaction contained plasmid, TNT lysate, amino acids, T7 polymerase, and Superase™ RNase inhibitor (Ambion).

The reactions were incubated at 30° C. for 3 hr, following which luciferase assay was performed using Steady Glo™ Luciferase assay kit (Promega). Average relative light units (RLUs) obtained were as follows:

Luciferase control plasmid=885

BI16=250

US Publ. No. 20060115493 recombinant segment M=29, (Luciferase under the control of a 5′ HCV IRES sequence)

The data indicate that the use of HCV TLS enables significantly better expression than HCV IRES alone.

### Example 3

### In Vitro Synthesis of Recombinant Single Strand RNA

In US Patent Publ. No. 20060115493, single-strand RNA (herein referred to as “ssRNA”) was prepared by using DuraScribe T7 polymerase kit (Epicentre, Madison, Wis.). In such transcription reactions, plasmids that carry the recombinant segments were first linearized by restriction enzyme (RE) digestion, using a restriction site downstream of the recombinant segments. Linearized plasmids were purified and then used as template for in vitro transcription. However, the 3′-ends of such transcripts carry extensions of several irrelevant bases, so that transcript ends were not identical to wild-type segment terminal sequences. Moreover, the use of T7 polymerase in transcription generates RNA with at least one additional base on the 5′ end. Therefore the 5′ end of such transcripts were not identical to wild type phi-8 sequence. Although that procedure is useful for producing ssRNA for the subsequent launch of conventional rdsRP/rdsRN, the method was modified for preparing RPC's.

To correct the problem, ssRNA employed in the present study is synthesized in vitro using PCR-generated fragments encoding BI10 and BI8 as templates. PCR fragments are used rather than restriction enzyme linearized plasmids so as to generate 3′-ends that are identical to wild-type phi-8 or that carry modifications of the terminal sequences described above. The primers used are designed such that the forward primer carries an SP6 promoter sequence and places the start of transcription at the 5′-guanidine of BI8 and BI10; thus the same forward primer is used to amplify BI8 and BI10 and has the following sequence: 5′-GATTACAGATCTATTTAGGTGACACTATAG-3′ (SEQ ID NO:12). The reverse primers for BI8 and BI10 are designed such that the resulting PCR fragment is amplified with or without a 9 base extension on the 3′ end that is thought to improve replication. The primers without the 9 base extension result in recombinant segments with wild type ends. Thus, 2 reverse primers are designed for each recombinant segment as follows, with 9 base extensions shown in bold italics: BI8 reverse with extension

(SEQ ID nO: 13)
BI8 reverse with extension
5′- [symbol] gaagtggaggccgaagcctcactcc-3′;
(SEQ ID NO: 14)
BI8 reverse wt
5′-gaagtggaggccgaagcctcactcc-3′;
(SEQ ID NO: 15)
BI10 reverse with extension
5′- [symbol] gaagcggaggaccgaagtcctcactcc-3′;
(SEQ ID NO: 16)
BI10 reverse wt
5′-gaagcggaggaccgaagtcctcactcc-3′.

To create blunt ends, PCR is performed using Accuprime pfx polymerase (Invitrogen, Carlsbad, Calif.), and plasmids harboring BI8 and BI10 served as template. Successful amplification of BI10 and BI8 is verified by agarose gel electrophoresis followed by purification of the PCR reaction using QIAquick PCR purification kit (Qiagen, Valencia, Calif., cat no. 28104). The concentration of the PCR-generated fragments encoding BI10 and BI8 is determined by spectrophotometry.

Durascribe SP6 (Epicentre) rather than Durascribe T7 polymerase is used for in vitro transcription because it generates RNA with 5′ terminal sequence that is identical to wild type phi-8. ssRNA is synthesized using DuraScribe SP6 polymerase kit according to the manufacturer's instructions using PCR-generated DNA encoding BI10 and BI8. The transcription reactions are treated with DNase Ito remove the template DNA and the reaction is purified using MEGAclear^{R} kit according to manufacturer's instructions (Ambion, Austin, Tex., Cat no. 1909). The synthetic ssRNA transcripts encoding BI10 and BI8 are verified for size in a 1% denaturing gel and the RNA concentrations are determined by spectrophotometry.

### Example 4

### Production of RPCs in TOP10 E. coli

Electrocompetent TOP10 cells (Invitrogen, Carlsbad, Calif.) were electroporated with the following mixture: 40 units Superase RNase inhibitor (Ambion)+1.0 μg pLM2653 (a plasmid that encodes phi-8 segment L)+20.0 μg BI10 ssRNA+20.0 μg BI8 ssRNA. Electroporation was conducted using a Gene-Pulser set at 200Ω, 25 mcF and 1.8 kV (BioRad, Hercules, Calif.) and the cells were allowed to recover by shaking in SOC medium (Invitrogen, Carlsbad, Calif., cat #15544-034) for 2 hr at 28° C. Subsequently, the cells were spun at 8000 rpm for 5 min, the SOC medium decanted, and the pellet resuspended in 5 ml Tryptic soy broth supplemented with 50 μg/ml kanamycin, 100 μg/ml carbenicillin, and 2 mM MnCl₂. The culture was incubated with shaking for 24 hr at 28° C., following which the cells were spun down as above, and finally spread on TSA supplemented with 50 μg/ml kanamycin, 100 μg/ml carbenicillin, 2 mM MnCl₂, 40 μg/ml β-galactosidase, and 100 μM isopropyl-β-D-thiogalactopyranoside (IPTG). Plates were incubated at 28° C. until single clones appeared, usually within 24 hr.

A blue colorimetric reaction results from expression of lacZα encoded in BI10, whereas kanamycin resistance is conferred by BI8. To date, kanamycin resistant clones have been isolated in RNA transformants with or without 9 base extensions on the 3′ ends. However, LacZ⁺ blue clones have been isolated only in transformants in that carry RPCs with the 9-base extension on the 3′ ends of BI8 and BI10. That observation indicates that the addition of 9-base extensions to the 3′ end of recombinant segments improves mRNA transcription in bacteria.

Single clones were screened for the presence of full length BI8 and BI10 by RT-PCR using primers specific for the amplification of either (−ve)- or (+ve)-strand RNA to recover cDNA's encoding BI10 and BI8. Recall that (−ve)-strand synthesis occurs only after uptake of all three genomic segments by the procapsid (Mindich, Microbiol. Mol. Biol. Rev. 63: 149-160 (1999)). Products of RT-PCR were size-verified by 1% agarose gel electrophoresis and the sequences were confirmed by nucleotide sequencing.

Another unexpected finding was that strains harboring RPCs with the appropriate PCR signal, nucleotide sequence, restriction endonuclease digestion pattern of pLM2653, could be frozen in 0.5 ml aliquots of TSB containing 30% glycerol at −80° C. Frozen stocks are easily revived in liquid or solid media supplemented as described above and growing at 28° C. for 18-24 hr.

Taken together, the data show that RPCs produce higher levels of mRNA in bacterial host strains and are more stable in storage, and hence display properties that will enable the production of stable recombinant bacterial strains that express passenger proteins.

### Example 5

### Purification of Replication-Proficient dsRC

US Patent Publ. No. 20060115493 provides sucrose gradient centrifugation as a method of isolating and purifying rdsRN from bacterial strains. However, sucrose gradient centrifugation is very inefficient, resulting in yields of rdsRN that virtually undetectable by immunoblot. It was necessary, therefore, to devise a method for purification of RPCs, which may be utilized for expression of recombinant proteins directly in eukaryotic cells in vitro and as vaccines in animal studies.

To purify capsids, single clones carrying RPCs were grown at 28° C. in TSB supplemented with appropriate antibiotics. Cultures were started at OD₆₀₀˜0.1 and cells were harvested during exponential growth (i.e. OD₆₀₀ 0.8). Cells were harvested by centrifugation at 8000 rpm for 5 min. The cells were rinsed with TES buffer (10 mM Tris-HCL (pH 7.5), 1 mM EDTA and 100 mM NaCl) and spun as above. The supernatant was discarded and the cells were resuspended cells in 500 μl of the TES buffer which included 40 U of Superase RNase inhibitor (Ambion). To lyse cells, 50 μl of 250 U/μl Ready-lyse lysozyme (Epicentre) was added and the cells were incubated at room temperature for 10-15 min with occasional swirling. Subsequently, the lysate was digested with 10 U of DNaseI at 37° C. for 30 min. The volume of the digest was brought up to 3 ml with a buffer containing 10 mM potassium phosphate, pH 7.5, and 1 mM magnesium chloride.

Next, the lysate was centrifuged at 12,000 rpm for 1 hour at 4° C. The spin was followed by centrifugation using a Steriflip 50 ml disposable vacuum system with a 22 μm pore size filter (Millipore Cat. No. SCGP00525). The supernatant was applied to a Centriprep centrifugal filter device that has a 50,000 molecular weight cutoff (Millipore, Billerica, Mass., cat No. 4323). Centriprep devices offer the advantage of a gentle filtration process, thereby, preventing sample denaturation. The device was centrifuged at 1500×g for 10 min and the RPCs are retained in the retentate. The filtrate was decanted and the retentate was further purified and concentrated by spinning twice at 1500×g for 5 min. The final volume after the purification process was ˜0.65 ml.

The presence of procapsids in the retentate was verified using standard electrophoresis procedures (Ausubel et al, supra, (1990)) that employed 15.5% (w/v) polyacrylamide gels containing 20% SDS (w/v), followed by an immunoblot. Immunodetection was achieved with rabbit polyclonal antibodies against phi-8 proteins P1 and P4. The results of the immunoblot show distinct immunoreactive P1 and P4 bands in the lanes in which the purified RPC samples were loaded.

### Transmission Electron Microscopy

Successful purification of RPCs was confirmed by transmission electron microscopy (herein referred to as “TEM”) using standard procedures on a fee-for-service basis at Johns Hopkins University Imaging Center, Baltimore Md. Purified RPCs were prepared for TEM by negative staining with 2% aqueous uranyl acetate. TEM confirmed the presence of large numbers of RPCs in purified samples, which displayed an appropriate size and structure that is similar to that of mature phi-8 nucleocapsids.

The ability of RPCs carrying BI10 to synthesize RNA is tested in vitro in a buffer that contains, 50 mM Tris-HCl (pH 8.2), 3 mM MgCl₂, 100 mM ammonium acetate, 20 mM NaCl, 5 mM KCl, 5 mM Dithiothreitol (DTT), 1 mM ATP, 1 mM CTP, 1 mM GTP, 1 mM UTP, 5% Polyethylene glycol 4000, 2 units Superase RNase Inhibitor, and 2 μl purified RPC-BI10. The results of this study confirm that RPCs are more adept at expressing mRNA than conventional rdsRP/rdsRN.

### Example 6

### Transfection of Mammalian Cells

Purified nucleocapsids infect spheroplasts of Pseudomonas syringae by binding to the plasma membrane followed by internalization via an endocytic-like process that is voltage-dependent (Poranen et al., J Cell Biol, 147:671; 1999). By definition, spheroplasts lack virtually all of the cell wall and, therefore, resemble mammalian cells. Therefore, purified RPCs transit across charged mammalian cell membranes, and express passenger genes. In addition to the direct method of crossing membranes, other non-passive methods of transfection, such as, protein transfection, particle bombardment, and electroporation are explored.

### Direct Application of RPCs to Mammalian Cells

To test the above hypothesis, purified RPCs that express BI8 and BI10 (i.e. fire fly luciferase) are used to transfect HeLa or HepG2 cells. Twenty-four hours before transfection, 2×10⁵ cells (Invitrogen, Carlsbad, Calif.) are seeded in 6-well plates and grown in DMEM+10% Fetal Bovine Serum (FBS) (Invitrogen)+1% Antibiotic/Antimycotic solution (Invitrogen) to 75% confluence at 37° C., 5% CO₂. The cells are washed twice with PBS and transfection with purified RPCs is conducted in serum free DMEM, using about 100 capsids per cell. As a positive control, cells are transfected with 4 μg of BI8 and BI10 RNA transcripts using TransIT mRNA transfection kit (Minis, Madison, Wis.). Transfection is allowed to proceed for 12-18 hr, following which the cells are lysed and luminescence is measured by using Steady-glo luciferase assay kit according to the manufacturer's instructions (Promega, Madison, Wis.).

### Non-Passive Methods of Transfection

Protein transfection may be achieved by the use of a protein transfection kit such as TransPass P (New England Biolabs). TransPass P reagent forms a non-covalent association with the protein that protects the protein from degradation upon endocytosis. Alternatively, the Proteojuice reagent (Novagen Cat. No. 71281-3) can be used. The Proteojuice reagent acts in the same manner. For example, Proteojuice was used to deliver and express recombinant simian/human immunodeficiency virus (SHIV) envelop to BHK-21 cells. The cells were lysed 24 hours after transfection and expression was confirmed by Western blot. A band of approximately 160 kb was observed from cells that were treated with the SHIV/RPCs but not from cells that were transfected with procapsids or luciferase-expressing RPCs.

Particle bombardment is commonly used to deliver DNA into cells by employing a gene gun, such as Helios™ (Bio-Rad). DNA is ethanol-precipitated onto fine gold particles (˜1 μm diameter), and is delivered using helium gas at approximately 400 psi. The method is adapted by coating gold particles with RPCs using polyethylene glycol-8000. RPC-coated gold particles are applied to gold coat tubing, dried with nitrogen, and cut down to cartridge size. The cartridges may then be used for delivery of RPCs-coated gold particles by a gene gun.

Transfection of mammalian cells with RPCs is also achieved by electroporation. The application of an electric pulse to mammalian cells perturbs the phospholipid bilayer of cell membranes resulting in the formation of temporary pores through which polar particles (that is RPCs) are driven.

### Example 7

### Exemplary Sequences for Recombinant Segment-S (BI10)

The following are exemplary sequences that may be used in the practice of the invention for construction of recombinant segment-S.

SP6 promoter

| | |
|---|---|
| ATTTAGGTGACACTATA | (SEQ ID NO: 17) |

connect to phi-8 S pac

Bacteriophage phi-8 segment-S pac (1-187 by of GenBank accession No. AF226853):

(SEQ ID NO: 18)
gaaattttcaaatcttttgactatttcgctggcatagctcttcggagtga
agccttccctgaaaggcgcgaaggtccccaccagctcggggtgattcgtg
acatttcctgggatctcggagtcagctttgtctctaggagactgagcgtt
cggtctcaggtttaaactgagattgaggataaagaca

connect to gene8

Bacteriophage phi-8 gene 8 (nucleotides 188-1288 of GenBank accession No. AF226853)

(SEQ ID NO: 19)
atgggtagaatctttcaactgttgatgcgcttaggcgttaaacagggtgc
agcaagtgttggtaaagccgggatcgatgctggtagcaagcgattgctcc
agcagatcatgtccaaagacggtgctattcagctgtctaaggcactcggt
ttcaccgctgtggagcagatgtcgagtgaagtgctcgaagcgtatctcta
tgagatcgttgagcatcttctgctcgtcgacgaggccacgttggccgatg
cgcttatggcgtgtatcaccgatgcaggtgatatcgccattgagcgtctg
cttccttccgtagaggatgtcgacaaaggcgaggcgcttgccgccacgct
gactgtcgtcttggctctcttctcgatgaacaaagaacaagctgaagagc
ttaaacgttcgatggcatcgaaaggcttgagtccggaccgggttaccctc
ggaggacagaccctgttgaccgtcaagtccactggtactggcctgacaga
gtatgacgctcaaggcaagaatggcgtccctcgcgggatgtctgctaaca
agcgtactgcattgttcttcgtgctgtacacagtgatcagtacttcctgg
tccgtatacgatcactatggtgaggttaaagctggtctcgcacgaggcga
gctacctcccagtgctgatcgtgttgaattgcgggcccccggttcctccg
taagtgcgatcgagcgtgagacacaacgcgcactgcaagaagaacagccg
cgtgcattgccttcgggcagccgcaccgcggaacgggttgctgggccgac
gcagggtgatgtccccgtgctcacacctccgccaggtcgattcaccttca
ccggtgagggcgaccatcgtcccgatttcgcacaactcgctcgccagaac
gacactgatggcgttgtgcggatcattgaactggatcgcattccagatgc
aaggaaaatattagtcgatggtgaccatgactacttgctggacgccgctc
aacagcgcgtcgctgccgatatcggggtatcgcccgagtcagtaggtcga
ttcgctgctctggtagccagtatcatcaacgcgaaggagaagcgttcgtg
a

connect to RBS

Shine-Dalgarno sequence

| | |
|---|---|
| aggaggacagct | (SEQ ID NO: 20) |

connect to lacZα

lacZα (from pCR1000, Invitrogen)

(SEQ ID NO: 21)
ATGACCATGATTACGCCAAGCTCAATACGACTCACTATAGGGCCCGGTAC
CGAGCTCACTAGTTTAATTAAAAGCTTATCGGCCGAGGTGAGAAGGGTTT
CGATATCGAGAGAAACGGTTCTCACCGCGGCCGCGAATTCACTGGCCGTC
GTTTTACAACGTCGTGACTGGGAAAACCCTGGCGTTACCCAACTTAATCG
CCTTGCAGCACATCCCCCTTTCGCCAGCTGGCGTAATAGCGAAGAGGCCC
GCACCGATCGCCCTTCCCAACAGTTGCGCAGCCTGAATGGCGAATGGCGG
CCGC

connect to HCV 5′ TLS

Hepatitis C virus-5′ TLS (bases 36-372 of GenBank accession No. AJ242651)

(SEQ ID NO: 22)
atcactcccctgtgaggaactactgtcttcacgcagaaagcgtctagcca
tggcgttagtatgagtgtcgtgcagcctccaggaccccccctcccgggag
agccatagtggtctgcggaaccggtgagtacaccggaattgccaggacga
ccgggtcctttcttggatcaacccgctcaatgcctggagatttgggcgtg
cccccgcgagactgctagccgagtagtgttgggtcgcgaaaggccttgtg
gtactgcctgatagggtgcttgcgagtgccccgggaggtctcgtagaccg
tgcaccatgagcacgaatcctaaacctcaaagaaaaa

connect to RE

AscI and HpaI RE site:

| | |
|---|---|
| ggcgcgccgttaac | (SEQ ID NO: 23) |

connect to Kozak

Kozak sequence

| | |
|---|---|
| CGCCACCATG | (SEQ ID NO: 24) |

connect to humanized HcRed1

HcRed1 human codon optimized (from pHcRed1, Clontech, cat #632410)

(SEQ ID NO: 25)
GTGAGCGGCCTGCTGAAGGAGAGTATGCGCATCAAGATGTACATGGAGGG
CACCGTGAACGGCCACTACTTCAAGTGCGAGGGCGAGGGCGACGGCAACC
CCTTCGCCGGCACCCAGAGCATGAGAATCCACGTGACCGAGGGCGCCCCC
CTGCCCTTCGCCTTCGACATCCTGGCCCCCTGCTGCGAGTACGGCAGCAG
GACCTTCGTGCACCACACCGCCGAGATCCCCGACTTCTTCAAGCAGAGCT
TCCCCGAGGGCTTCACCTGGGAGAGAACCACCACCTACGAGGACGGCGGC
ATCCTGACCGCCCACCAGGACACCAGCCTGGAGGGCAACTGCCTGATCTA
CAAGGTGAAGGTGCACGGCACCAACTTCCCCGCCGACGGCCCCGTGATGA
AGAACAAGAGCGGCGGCTGGGAGCCCAGCACCGAGGTGGTGTACCCCGAG
AACGGCGTGCTGTGCGGCCGGAACGTGATGGCCCTGAAGGTGGGCGACCG
GCACCTGATCTGCCACCACTACACCAGCTACCGGAGCAAGAAGGCCGTGC
GCGCCCTGACCATGCCCGGCTTCCACTTCACCGACATCCGGCTCCAGATG
CTGCGGAAGAAGAAGGACGAGTACTTCGAGCTGTACGAGGCCAGCGTGGC
CCGGTACAGCGACCTGCCCGAGAAGGCCAACTGA

connect to restriction sites

NotI and Pact Restriction sites

| | |
|---|---|
| gcggccgcttaattaa | ((SEQ ID NO: 26) |

connect to HCV 3′ TLS

Hepatitis C virus-3′ TLS (bases 8539-8637 of GenBank accession No. AJ242651)

(SEQ ID NO: 27)
ggtggctccatcttagccctagtcacggctagctgtgaaaggtccgtgag
ccgcttgactgcagagagtgctgatactggcctctctgcagatcaagt

connect to segment S 3-prime end

phi-8 segment-S 3-prime polymerase binding site (bases 3081-3192 of GenBank accession No. AF226853) with 3′ extension shown in upper case

(SEQ ID NO: 28)
gcttagcggcaatcgaaccctccgataaggaggtttagcaaatccgcggc
tcttatgagctgtccgaaaggacaacccgaaagggggagtgaggacttcg
gtcctccgcttcGAAAATTTC

### Example 8

### Exemplary Sequences for Recombinant Segment-M (BI8)

The following are exemplary sequences that may be used in the practice of the invention for construction of recombinant segment-M.

SP6 Promoter

| | |
|---|---|
| ATTTAGGTGACACTATA | (SEQ ID NO: 17) |

Segment M pac sequence and ribosomal binding site of gene 10 (bases 1-262 of GenBank accession No. AF226852)

(SEQ ID NO: 29)
gaaattttcaaagtctttcggcaataagggtggaaatttcaaagagggtc
gagccgacgaacctctgtagaaccgggaagtgcctgtctttacttgcgag
agcaattgaactagggcagcaccgggggtcgataagcgcagaagtgaggc
gcggggattgaagcaaatcacctaagcgtaaacgacggacctcgagggtg
gcggagtctacataggatcccctagctactagacagaaaccattcctaac
aaggagatgcac

connect to FseI site

FseI site

| | |
|---|---|
| ggccggcc | (SEQ ID NO: 30) |

connect to Shine-Dalgarno

Shine Dalgarno sequence

| | |
|---|---|
| aggaggacagct | (SEQ ID NO: 20) |

connect to gene 12

Phi-8 gene 12 (GenBank accession No. AF226853)

(SEQ ID NO: 31)
atgcttaagatgctgctcgctacgcaaggtcttacgaccgcatccatcat
ggaaattctcactggttttgtggtgagacgggttcgactcgaggctcagc
ctttggtcgctagtatgatgccctccgttcgtgaggaggttgctcgagcg
ctcaacgacaaggcgtacaggcaagtactcgcaggggctggccaagttac
actccgtgtattcaacggggttggtcagctggagacaccacttgtcgaga
ttatcaaggatatcgcccgactgtcatcccctaccttcaagagcatgctc
gagaaggcggagagaggcgagaatcttggtagtatgaccgacgaactcgc
agagtccatagtggcagagttggttgcgctgatctcagccgatgcaaccg
acgtgacgacagcactctctgtcccaggcgctgacgtggagcgctaccgt
ttgatcgttgattggctcaggggtcacatcaagtcgattgagcaaaaaga
tctgttcccggacatcatcgatttcctggagtag

connect to FseI site

FseI site

| | |
|---|---|
| ggccggcc | (SEQ ID NO: 30) |

connect to SbfI site

SbfI

| | |
|---|---|
| cctgcagg | (SEQ ID NO: 32) |

connect to Shine-Dalgarno

Shine Dalgarno sequence

| | |
|---|---|
| aggaggacagct | (SEQ ID NO: 20) |

bla gene (Ampicillin resistance)

(SEQ ID NO: 33)
ATGAGTATTCAACATTTCCGTGTCGCCCTTATTCCCTTTTTTGCGGCATT
TTGCCTTCCTGTTTTTGCTCACCCAGAAACGCTGGTGAAAGTAAAAGATG
CTGAAGATCAGTTGGGTGCACGAGTGGGTTACATCGAACTGGATCTCAAC
AGCGGTAAGATCCTTGAGAGTTTTCGCCCCGAAGAACGTTTTCCAATGAT
GAGCACTTTTAAAGTTCTGCTATGTGGCGCGGTATTATCCCGTATTGACG
CCGGGCAAGAGCAACTCGGTCGCCGCATACACTATTCTCAGAATGACTTG
GTTGAGTACTCACCAGTCACAGAAAAGCATCTTACGGATGGCATGACAGT
AAGAGAATTATGCAGTGCTGCCATAACCATGAGTGATAACACTGCGGCCA
ACTTACTTCTGACAACGATCGGAGGACCGAAGGAGCTAACCGCTTTTTTG
CACAACATGGGGGATCATGTAACTCGCCTTGATCGTTGGGAACCGGAGCT
GAATGAAGCCATACCAAACGACGAGCGTGACACCACGATGCCTGTAGCAA
TGGCAACAACGTTGCGCAAACTATTAACTGGCGAACTACTTACTCTAGCT
TCCCGGCAACAATTAATAGACTGGATGGAGGCGGATAAAGTTGCAGGACC
ACTTCTGCGCTCGGCCCTTCCGGCTGGCTGGTTTATTGCTGATAAATCTG
GAGCCGGTGAGCGTGGGTCTCGCGGTATCATTGCAGCACTGGGGCCAGAT
GGTAAGCCCTCCCGTATCGTAGTTATCTACACGACGGGGAGTCAGGCAAC
TATGGATGAACGAAATAGACAGATCGCTGAGATAGGTGCCTCACTGATTA
AGCATTGGTAA

connect to SbfI site

SbfI

| | |
|---|---|
| cctgcagg | (SEQ ID NO: 32) |

connect to Shine-Dalgarno

Shine Dalgarno sequence

| | |
|---|---|
| aggaggacagct | (SEQ ID NO: 20) |

connect to Kan

Aminoglycoside phosphotransferase Kanamycin resistance protein (DQ851853)

(SEQ ID NO: 34)
atgagccatattcaacgggaaacgtcttgctctaggccgcgattaaattc
caacatggatgctgatttatatgggtataaatgggctcgcgataatgtcg
ggcaatcaggtgcgacaatctatcgattgtatgggaagcccgatgcgcca
gagttgtttctgaaacatggcaaaggtagcgttgccaatgatgttacaga
tgagatggtcagactaaactggctgacggaatttatgcctcttccgacca
tcaagcattttatccgtactcctgatgatgcatggttactcaccactgcg
atccccgggaaaacagcattccaggtattagaagaatatcctgattcagg
tgaaaatattgttgatgcgctggcagtgttcctgcgccggttgcattcga
ttcctgtttgtaattgtccttttaacagcgatcgcgtatttcgtctcgct
caggcgcaatcacgaatgaataacggtttggttgatgcgagtgattttga
tgacgagcgtaatggctggcctgttgaacaagtctggaaagaaatgcata
aacttttgccattctcaccggattcagtcgtcactcatggtgatttctca
cttgataaccttatttttgacgaggggaaattaataggttgtattgatgt
tggacgagtcggaatcgcagaccgataccaggatcttgccatcctatgga
actgcctcggtgagttttctccttcattacagaaacggctttttcaaaaa
tatggtattgataatcctgatatgaataaattgcagtttcatttgatgct
cgatgagtttttctaa

connect to HCV 5′ TLS

Hepatitis C virus-5′ TLS (bases 36-372 of GenBank accession No. AJ242651)

(SEQ ID NO: 22)
atcactcccctgtgaggaactactgtcttcacgcagaaagcgtctagcca
tggcgttagtatgagtgtcgtgcagcctccaggaccccccctcccgggag
agccatagtggtctgcggaaccggtgagtacaccggaattgccaggacga
ccgggtcctttcttggatcaacccgctcaatgcctggagatttgggcgtg
cccccgcgagactgctagccgagtagtgttgggtcgcgaaaggccttgtg
gtactgcctgatagggtgcttgcgagtgccccgggaggtctcgtagaccg
tgcaccatgagcacgaatcctaaacctcaaagaaaaa

connect to RE site

RE site (AscI and HpaI)

| | |
|---|---|
| ggcgcgccgttaac | (SEQ ID NO: 23) |

connect to Kozak

Kozak sequence

| | |
|---|---|
| gccgccaccatgggc | (SEQ ID NO: 35) |

go to luciferase

Luciferase (from gWIZ-luciferase, Genlantis, cat #P030200)

(SEQ ID NO: 36)
ATGGAAGACGCCAAAAACATAAAGAAAGGCC1951CGGCGCCATTCTATC
CGCTGGAAGATGGAACCGCTGGAGAGCAACTGCAT2001AAGGCTATGAA
GAGATACGCCCTGGTTCCTGGAACAATTGCTTTTACAGA2051TGCACAT
ATCGAGGTGGACATCACTTACGCTGAGTACTTCGAAATGTCCG2101TTC
GGTTGGCAGAAGCTATGAAACGATATGGGCTGAATACAAATCACAGA215
1ATCGTCGTATGCAGTGAAAACTCTCTTCAATTCTTTATGCCGGTGTTGG
G2201CGCGTTATTTATCGGAGTTGCAGTTGCGCCCGCGAACGACATTTA
TAATG2251AACGTGAATTGCTCAACAGTATGGGCATTTCGCAGCCTACC
GTGGTGTTC2301GTTTCCAAAAAGGGGTTGCAAAAAATTTTGAACGTGC
AAAAAAAGCTCCC2351AATCATCCAAAAAATTATTATCATGGATTCTAA
AACGGATTACCAGGGAT2401TTCAGTCGATGTACACGTTCGTCACATCT
CATCTACCTCCCGGTTTTAAT2451GAATACGATTTTGTGCCAGAGTCCT
TCGATAGGGACAAGACAATTGCACT2501GATCATGAACTCCTCTGGATC
TACTGGTCTGCCTAAAGGTGTCGCTCTGC2551CTCATAGAACTGCCTGC
GTGAGATTCTCGCATGCCAGAGATCCTATTTTT2601GGCAATCAAATCA
TTCCGGATACTGCGATTTTAAGTGTTGTTCCATTCCA2651TCACGGTTT
TGGAATGTTTACTACACTCGGATATTTGATATGTGGATTTC2701GAGTC
GTCTTAATGTATAGATTTGAAGAAGAGCTGTTTCTGAGGAGCCTT2751C
AGGATTACAAGATTCAAAGTGCGCTGCTGGTGCCAACCCTATTCTCCTT2
801CTTCGCCAAAAGCACTCTGATTGACAAATACGATTTATCTAATTTAC
ACG2851AAATTGCTTCTGGTGGCGCTCCCCTCTCTAAGGAAGTCGGGGA
AGCGGTT2901GCCAAGAGGTTCCATCTGCCAGGTATCAGGCAAGGATAT
GGGCTCACTGA2951GACTACATCAGCTATTCTGATTACACCCGAGGGGG
ATGATAAACCGGGCG3001CGGTCGGTAAAGTTGTTCCATTTTTTGAAGC
GAAGGTTGTGGATCTGGAT3051ACCGGGAAAACGCTGGGCGTTAATCAA
AGAGGCGAACTGTGTGTGAGAGG3101TCCTATGATTATGTCCGGTTATG
TAAACAATCCGGAAGCGACCAACGCCT3151TGATTGACAAGGATGGATG
GCTACATTCTGGAGACATAGCTTACTGGGAC3201GAAGACGAACACTTC
TTCATCGTTGACCGCCTGAAGTCTCTGATTAAGTA3251CAAAGGCTATC
AGGTGGCTCCCGCTGAATTGGAATCCATCTTGCTCCAAC3301ACCCCAA
CATCTTCGACGCAGGTGTCGCAGGTCTTCCCGACGATGACGCC3351GGT
GAACTTCCCGCCGCCGTTGTTGTTTTGGAGCACGGAAAGACGATGAC340
1GGAAAAAGAGATCGTGGATTACGTCGCCAGTCAAGTAACAACCGCGAAA
A3451AGTTGCGCGGAGGAGTTGTGTTTGTGGACGAAGTACCGAAAGGTC
TTACC3501GGAAAACTCGACGCAAGAAAAATCAGAGAGATCCTCATAAA
GGCCAAGAA3551GGGCGGAAAGATCGCCGTGTAA

connect to RE

RE sites (NotI and Pact)

| | |
|---|---|
| gcggccgcttaattaa | (SEQ ID NO: 26) |

connect to HCV 3′ TLS

Hepatitis C virus-3′ TLS (bases 8539-8637 of GenBank accession No. AJ242651)

(SEQ ID NO: 27)
ggtggctccatcttagccctagtcacggctagctgtgaaaggtccgtgag
ccgcttgactgcagagagtgctgatactggcctctctgcagatcaagt

connect to phi-8 segment M 3-prime end

Phi-8 segment M 3-prime Polymerase binding site (bases 4677-4741 of GenBank accession No. AF226852) with 3′ extension shown in upper case:

(SEQ ID NO: 37)
actgttgataaacaggacccggaagggtaacccgagagggggagtgaggc
ttcggcctccacttcGAAAATTTC

### Example 9

### Construction of Recombinant Segments that Express Adjuvant Activity

Recombinant segments for use in generating replication proficient rdsRN were designed such that each segment carries a prokaryotic and eukaryotic cassette. The objective was to construct ready-to-use capsids that express adjuvants of interest such that they can simply be mixed with other capsids that express immunogen and employed in vaccination. The recombinant segments were designed to express adjuvants such as IL-2, West Nile virus NS3 protease, and CTLA-4 antagonist. IL-2 functions in vivo to maintain self tolerance and is particularly useful in cancer immunotherapy. NS3 promotes caspase-8 activation thus resulting in apoptosis. Apoptosis promotes cross presentation of antigen by dendritic cells, which, in turn, results in improved immunostimulation.

Genes encoding IL-2, NS3, and CTLA-4 were generated by synthetic DNA procedures and inserted into plasmid pJ53 (DNA 2.0). As shown in the schematic below, the genes were configured such that IL-2 and NS3 are expressed on recombinant segment S, whereas CTLA-4 antagonist is expressed from recombinant segment M. Recombinant segment S encodes, starting at the 5′-end: SP6 promoter→phi-8 pac S→phi-8 gene 8→lacZα→HCV 5′TLS→mouse IL-2→ATPase 3′ UTR→West Nile virus NS3 protease→HCV 3′TLS→phi-8 segment S 3′UTR. Recombinant segment M encodes, starting at the 5′-end: SP6 promoter→phi-8 pacM→phi-8 gene 12→bla (Amp^{R})→agh (Kan^{g})→HCV 5′TLS→CTLA-4 antagonist→HCV 3′TLS→phi-8 segment M 3′UTR.

The complete sequences of recombinant segments S and M are shown below.

Adjuvant capsid recombinant segment S

SP6 promoter

| | |
|---|---|
| ATTTAGGTGAC ACTATA | (SEQ ID NO: 17) |

connect to phi-8 S pac

Bacteriophage phi-8 segment-S pac (1-187 by of GenBank accession No. AF226853):

(SEQ ID NO: 18)
gaaattttcaaatcttttgactatttcgctggcatagctcttcggagtga
agccttccctgaaaggcgcgaaggtccccaccagctcggggtgattcgtg
acatttcctgggatctcggagtcagctttgtctctaggagactgagcgtt
cggtctcaggtttaaactgagattgaggataaagaca

connect to gene8

Bacteriophage phi-8 gene 8 (nucleotides 188-1288 of GenBank accession No. AF226853)

(SEQ ID NO: 19)
atgggtagaatctttcaactgttgatgcgcttaggcgttaaacagggtgc
agcaagtgttggtaaagccgggatcgatgctggtagcaagcgattgctcc
agcagatcatgtccaaagacggtgctattcagctgtctaaggcactcggt
ttcaccgctgtggagcagatgtcgagtgaagtgctcgaagcgtatctcta
tgagatcgttgagcatcttctgctcgtcgacgaggccacgttggccgatg
cgcttatggcgtgtatcaccgatgcaggtgatatcgccattgagcgtctg
cttccttccgtagaggatgtcgacaaaggcgaggcgcttgccgccacgct
gactgtcgtcttggctctcttctcgatgaacaaagaacaagctgaagagc
ttaaacgttcgatggcatcgaaaggcttgagtccggaccgggttaccctc
ggaggacagaccctgttgaccgtcaagtccactggtactggcctgacaga
gtatgacgctcaaggcaagaatggcgtccctcgcgggatgtctgctaaca
agcgtactgcattgttcttcgtgctgtacacagtgatcagtacttcctgg
tccgtatacgatcactatggtgaggttaaagctggtctcgcacgaggcga
gctacctcccagtgctgatcgtgttgaattgcgggcccccggttcctccg
taagtgcgatcgagcgtgagacacaacgcgcactgcaagaagaacagccg
cgtgcattgccttcgggcagccgcaccgcggaacgggttgctgggccgac
gcagggtgatgtccccgtgctcacacctccgccaggtcgattcaccttca
ccggtgagggcgaccatcgtcccgatttcgcacaactcgctcgccagaac
gacactgatggcgttgtgcggatcattgaactggatcgcattccagatgc
aaggaaaatattagtcgatggtgaccatgactacttgctggacgccgctc
aacagcgcgtcgctgccgatatcggggtatcgcccgagtcagtaggtcga
ttcgctgctctggtagccagtatcatcaacgcgaaggagaagcgttcgtg
a

connect to FseI

FseI site

| | |
|---|---|
| ggccggcc | (SEQ ID NO: 30) |

connect to Shine-Dalgarno

Shine-Dalgarno sequence

| | |
|---|---|
| aggaggacagct | (SEQ ID NO: 20) |

connect to lacZα

lacZα (from pCR1000, Invitrogen)

(SEQ ID NO: 21)
ATGACCATGATTACGCCAAGCTCAATACGACTCACTATAGGGCCCGGTAC
CGAGCTCACTAGTTTAATTAAAAGCTTATCGGCCGAGGTGAGAAGGGTTT
CGATATCGAGAGAAACGGTTCTCACCGCGGCCGCGAATTCACTGGCCGTC
GTTTTACAACGTCGTGACTGGGAAAACCCTGGCGTTACCCAACTTAATCG
CCTTGCAGCACATCCCCCTTTCGCCAGCTGGCGTAATAGCGAAGAGGCCC
GCACCGATCGCCCTTCCCAACAGTTGCGCAGCCTGAATGGCGAATGGCGG
CCGC

connect to FseI and SbfI

FseI and SbfI sites

| | |
|---|---|
| ggccggcccctgcagg | (SEQ ID NOS: 79) |

connect to HCV 5′ TLS

Hepatitis C virus-5′ TLS (bases 36-372 of GenBank accession No. AJ242651)

(SEQ ID NO: 22)
atcactcccctgtgaggaactactgtcttcacgcagaaagcgtctagcca
tggcgttagtatgagtgtcgtgcagcctccaggaccccccctcccgggag
agccatagtggtctgcggaaccggtgagtacaccggaattgccaggacga
ccgggtcctttcttggatcaacccgctcaatgcctggagatttgggcgtg
cccccgcgagactgctagccgagtagtgttgggtcgcgaaaggccttgtg
gtactgcctgatagggtgcttgcgagtgccccgggaggtctcgtagaccg
tgcaccatgagcacgaatcctaaacctcaaagaaaaa

connect to RE

AscI and HpaI RE site:

| | |
|---|---|
| ggcgcgccgttaac | (SEQ ID NO: 23) |

connect to Kozak

Kozak sequence

| | |
|---|---|
| CGCCACCATG | (SEQ ID NO: 24) |

connect to mouse IL-2

Mouse IL-2

(SEQ ID NO: 38)
ATGTACTCTATGCAGCTGGCATCTTGTGTTACTTTGACACTGGTGCTTCT
GGTCAACTCAGCCCCAACTTCATCTTCCACCAGTAGCAGTACCGCCGAGG
CTCAACAGCAGCAGCAACAGCAGCAACAACAACAGCAGCACCTGGAGCAA
CTGCTCATGGACCTGCAAGAGTTGCTGTCAAGGATGGAAAACTATAGAAA
CCTTAAACTGCCACGGATGCTGACCTTCAAGTTCTACCTGCCCAAGCAAG
CAACCGAACTCAAGGATCTGCAATGCCTTGAGGATGAACTGGGCCCCCTG
CGGCATGTGCTGGATCTGACGCAGTCTAAATCTTTCCAGCTCGAAGACGC
AGAAAATTTCATTAGCAACATTCGGGTGACTGTGGTAAAATTGAAAGGGT
CTGACAACACTTTTGAGTGCCAGTTCGACGACGAGTCAGCCACCGTCGTG
GACTTTTTGAGACGCTGGATCGCCTTTTGCCAGTCAATCATATCCACTAG
TCCCCAG
(SEQ ID NO: 39)
MYSMQLASCVTLTLVLLVNSAPTSSSTSSSTAEAQQQQQQQQQQQQHLEQ
LLMDLQELLSRMENYRNLKLPRMLTFKFYLPKQATELKDLQCLEDELGPL
RHVLDLTQSKSFQLEDAENFISNIRVTVVKLKGSDNTFECQFDDESATVV
DFLRRWIAFCQSIISTSPQ

Stop

TAG

connect to ATPase 3′UTR

Rat F1-ATPase 13 subunit mRNA, 3′ UTR (accession No. M57634)

(SEQ ID NO: 40)
gggcccttcagccaaacacaacagcactctgcactgacctccatgctgag
agctcagtttgccatgtaggccacacaagagccttgattgaagatgtgat
gttctctctgaagagtatttaaagttttcaataaagtatataccctc

connect to Kozak

Kozak sequence

| | |
|---|---|
| CGCCACCATG | (SEQ ID NO: 24) |

connect to NS3

West Nile virus NS3 protease (GenBank accession No. DQ176637)

(SEQ ID NO: 41)
ATGGGCGGAGTGCTCTGGGATACTCCGAGCCCTAAGGAGTACAAAAAGGG
CGACACTACAACTGGAGTGTATAGGATCATGACAAGAGGATTGCTCGGGA
GTTATCAAGCGGGCGCAGGGGTGATGGTCGAGGGGGTGTTTCATACTCTC
TGGCACACCACCAAAGGGGCTGCCCTCATGAGCGGTGAGGGGCGGCTGGA
TCCCTACTGGGGGAGCGTCAAAGAGGACAGATTGTGCTATGGCGGGCCGT
GGAAGCTGCAGCATAAGTGGAACGGCCAAGACGAAGTGCAGATGATCGTG
GTGGAGCCTGGCAAAAACGTGAAAAACGTTCAGACCAAACCAGGCGTCTT
CAAAACACCAGAAGGGGAAATCGGCGCTGTCACACTGGATTTTCCTACGG
GCACCTCAGGTTCCCCCATAGTCGACAAAAATGGAGACGTCATCGGCCTG
TATGGAAACGGGGTCATTATGCCGAACGGCTCCTATATCTCAGCGATCGT
CCAGGGGGAGCGGATGGACGAGCCAATCCCGGCAGGGTTCGAGCCGGAAA
TGCTTCGGAAGAAACAGATAACC
(SEQ ID NO: 42)
MGGVLWDTPSPKEYKKGDTTTGVYRIMTRGLLGSYQAGAGVMVEGVFHTL
WHTTKGAALMSGEGRLDPYWGSVKEDRLCYGGPWKLQHKWNGQDEVQMIV
VEPGKNVKNVQTKPGVFKTPEGEIGAVTLDFPTGTSGSPIVDKNGDVIGL
YGNGVIMPNGSYISAIVQGERMDEPIPAGFEPEMLRKKQIT

Linker

| | |
|---|---|
| GGATCCTCCTCATCCGGG | (SEQ ID NO: 43) |
| GSSSSG | (SEQ ID NO: 44) |

HIS tag

| | |
|---|---|
| CACCACCACCATCACCAC | (SEQ ID NO: 45) |
| HHHHHH | (SEQ ID NO: 46) |

Stop

TAA

connect to restriction sites

NotI and Pact Restriction sites

| | |
|---|---|
| gcggccgcttaattaa | (SEQ ID NO: 26) |

connect to HCV 3′ TLS

Hepatitis C virus-3′ TLS (bases 8539-8637 of GenBank accession No. AJ242651)

(SEQ ID NO: 27)
ggtggctccatcttagccctagtcacggctagctgtgaaaggtccgtgag
ccgcttgactgcagagagtgctgatactggcctctctgcagatcaagt

connect to segment S 3-prime end

phi-8 segment-S 3-prime polymerase binding site (bases 3081-3192 of GenBank accession No. AF226853) with 3′ extension shown in upper case

(SEQ ID NO: 28)
gcttagcggcaatcgaaccctccgataaggaggtttagcaaatccgcggc
tcttatgagctgtccgaaaggacaacccgaaagggggagtgaggacttcg
gtcctccgcttcGAAAATTTC

Adjuvant capsid recombinant segment M

SP6 Promoter

| | |
|---|---|
| ATTTAGGTGACACTATA | (SEQ ID NO: 17) |

Segment M pac sequence and ribosomal binding site of gene 10 (bases 1-262 of GenBank accession No. AF226852)

(SEQ ID NO: 29)
gaaattttcaaagtctttcggcaataagggtggaaatttcaaagagggtc
gagccgacgaacctctgtagaaccgggaagtgcctgtctttacttgcgag
agcaattgaactagggcagcaccgggggtcgataagcgcagaagtgaggc
gcggggattgaagcaaatcacctaagcgtaaacgacggacctcgagggtg
gcggagtctacataggatcccctagctactagacagaaaccattcctaac
aaggagatgcac

connect to FseI site

FseI site

| | |
|---|---|
| ggccggcc | (SEQ ID NO: 30) |

connect to Shine-Dalgarno

Shine Dalgarno sequence

| | |
|---|---|
| gagaggacagct | (SEQ ID NO: 47) |

connect to gene 12

Phi-8 gene 12 (GenBank accession No. AF226853)

(SE ID NO: 31)
atgcttaagatgctgctcgctacgcaaggtcttacgaccgcatccatcat
ggaaattctcactggttttgtggtgagacgggttcgactcgaggctcagc
ctttggtcgctagtatgatgccctccgttcgtgaggaggttgctcgagcg
ctcaacgacaaggcgtacaggcaagtactcgcaggggctggccaagttac
actccgtgtattcaacggggttggtcagctggagacaccacttgtcgaga
ttatcaaggatatcgcccgactgtcatcccctaccttcaagagcatgctc
gagaaggcggagagaggcgagaatcttggtagtatgaccgacgaactcgc
agagtccatagtggcagagttggttgcgctgatctcagccgatgcaaccg
acgtgacgacagcactctctgtcccaggcgctgacgtggagcgctaccgt
ttgatcgttgattggctcaggggtcacatcaagtcgattgagcaaaaaga
tctgttcccggacatcatcgatttcctggagtag

connect to FseI site

FseI site

| | |
|---|---|
| ggccggcc | (SEQ ID NO: 30) |

connect to SbfI site

SbfI

| | |
|---|---|
| cctgcagg | (SEQ ID NO: 32) |

connect to Shine-Dalgarno

Shine Dalgarno sequence

| | |
|---|---|
| aggaggacagct | (SEQ ID NO: 20) |

bla gene (Ampicillin resistance)

(SEQ ID NO: 33)
ATGAGTATTCAACATTTCCGTGTCGCCCTTATTCCCTTTTTTGCGGCATT
TTGCCTTCCTGTTTTTGCTCACCCAGAAACGCTGGTGAAAGTAAAAGATG
CTGAAGATCAGTTGGGTGCACGAGTGGGTTACATCGAACTGGATCTCAAC
AGCGGTAAGATCCTTGAGAGTTTTCGCCCCGAAGAACGTTTTCCAATGAT
GAGCACTTTTAAAGTTCTGCTATGTGGCGCGGTATTATCCCGTATTGACG
CCGGGCAAGAGCAACTCGGTCGCCGCATACACTATTCTCAGAATGACTTG
GTTGAGTACTCACCAGTCACAGAAAAGCATCTTACGGATGGCATGACAGT
AAGAGAATTATGCAGTGCTGCCATAACCATGAGTGATAACACTGCGGCCA
ACTTACTTCTGACAACGATCGGAGGACCGAAGGAGCTAACCGCTTTTTTG
CACAACATGGGGGATCATGTAACTCGCCTTGATCGTTGGGAACCGGAGCT
GAATGAAGCCATACCAAACGACGAGCGTGACACCACGATGCCTGTAGCAA
TGGCAACAACGTTGCGCAAACTATTAACTGGCGAACTACTTACTCTAGCT
TCCCGGCAACAATTAATAGACTGGATGGAGGCGGATAAAGTTGCAGGACC
ACTTCTGCGCTCGGCCCTTCCGGCTGGCTGGTTTATTGCTGATAAATCTG
GAGCCGGTGAGCGTGGGTCTCGCGGTATCATTGCAGCACTGGGGCCAGAT
GGTAAGCCCTCCCGTATCGTAGTTATCTACACGACGGGGAGTCAGGCAAC
TATGGATGAACGAAATAGACAGATCGCTGAGATAGGTGCCTCACTGATTA
AGCATTGGTAA

connect to SbfI site

SbfI

| | |
|---|---|
| cctgcagg | (SEQ ID NO: 32) |

connect to Shine-Dalgarno

Shine Dalgarno sequence

| | |
|---|---|
| aggaggacagct | (SEQ ID NO: 20) |

connect to Kan

Aminoglycoside phosphotransferase Kanamycin resistance protein (DQ851853)

(SEQ ID NO: 34)
atgagccatattcaacgggaaacgtcttgctctaggccgcgattaaattc
caacatggatgctgatttatatgggtataaatgggctcgcgataatgtcg
ggcaatcaggtgcgacaatctatcgattgtatgggaagcccgatgcgcca
gagttgtttctgaaacatggcaaaggtagcgttgccaatgatgttacaga
tgagatggtcagactaaactggctgacggaatttatgcctcttccgacca
tcaagcattttatccgtactcctgatgatgcatggttactcaccactgcg
atccccgggaaaacagcattccaggtattagaagaatatcctgattcagg
tgaaaatattgttgatgcgctggcagtgttcctgcgccggttgcattcga
ttcctgtttgtaattgtccttttaacagcgatcgcgtatttcgtctcgct
caggcgcaatcacgaatgaataacggtttggttgatgcgagtgattttga
tgacgagcgtaatggctggcctgttgaacaagtctggaaagaaatgcata
aacttttgccattctcaccggattcagtcgtcactcatggtgatttctca
cttgataaccttatttttgacgaggggaaattaataggttgtattgatgt
tggacgagtcggaatcgcagaccgataccaggatcttgccatcctatgga
actgcctcggtgagttttctccttcattacagaaacggctttttcaaaaa
tatggtattgataatcctgatatgaataaattgcagtttcatttgatgct
cgatgagtttttctaa

connect to HCV 5′ TLS

Hepatitis C virus-5′ TLS (bases 36-372 of GenBank accession No. AJ242651)

(SEQ ID NO: 22)
atcactcccctgtgaggaactactgtcttcacgcagaaagcgtctagcca
tggcgttagtatgagtgtcgtgcagcctccaggaccccccctcccgggag
agccatagtggtctgcggaaccggtgagtacaccggaattgccaggacga
ccgggtcctttcttggatcaacccgctcaatgcctggagatttgggcgtg
cccccgcgagactgctagccgagtagtgttgggtcgcgaaaggccttgtg
gtactgcctgatagggtgcttgcgagtgccccgggaggtctcgtagaccg
tgcaccatgagcacgaatcctaaacctcaaagaaaaa

connect to RE site

RE site (AscI and HpaI)

| | |
|---|---|
| ggcgcgccgttaac | (SEQ ID NO: 23) |

connect to Kozak

Kozak sequence

| | |
|---|---|
| gccgccacc | (nucleotides 1-9 of SEQ ID NO: 35) |

go to CTLA-4 antagonist

CTLA-4 antagonist

(SEQ ID NO: 48)
ATGGCCTGTAATTGTCAACTGATGCAGGATACCCCCTTGCTTAAATTTCC
CTGCCCCCGACTGATCCTCCTCTTTGTTCTCCTCATACGCCTCTCTCAAG
TGTCTAGCGATGTAGACGAACAGCTGTCAAAAAGCGTGAAGGATAAAGTC
TTGCTGCCCTGTCGATACAACAGCCCGCACGAGGACGAATCCGAAGATCG
CATTTACTGGCAGAAGCACGACAAGGTGGTGCTCTCCGTGATCGCTGGCA
AGTTGAAAGTGGCTCCCGAGTATAAGAATCGAACTCTGTACGATAATACC
ACATACTCTTTGATTATTTTGGGCCTGGTTCTGTCTGATAGGGGGACCTA
TAGCTGCGTTGTGCAGAAGAAAGAGCGCGGGACATACGAAGTTAAGCACC
TTGCCCTCGTTAAGCTGTCTATAAAGGCTGACTTCAGCACACCGAATATT
ACTGAATCTGGGAACCCTAGCGCAGATACAAAAAGGATCACCTGCTTCGC
TTCCGGCGGGTTCCCTAAACCCCGGTTCTCATGGCTTGAGAATGGCCGGG
AGCTGCCAGGAATCAACACCACCATTTCACAGGACCCCGAGTCTGAACTC
TACACAATCAGCAGCCAACTGGATTTTAATACGACCCGAAATCACACAAT
TAAGTGTCTCATTAAATATGGAGATGCCCATGTTAGCGAAGACTTTACTT
GGGGCAGTTCCTCCTCAGGCTGCAAGCCCTGTATATGTACGGTGCCAGAG
GTCAGCTCTGTCTTTATATTCCCTCCTAAGCCTAAGGACGTTCTGACCAT
TACCCTCACTCCGAAGGTAACATGTGTTGTGGTGGATATCTCAAAGGACG
ACCCTGAGGTACAATTCTCCTGGTTTGTGGATGACGTGGAGGTTCACACG
GCTCAGACAAAACCACGGGAAGAACAGTTTAATTCAACGTTTCGCTCAGT
GAGTGAGCTCCCTATCATGCACCAGGACTGGTTGAATGGGAAGGAGTTCA
AATGTCGCGTTAACAGCGCAGCATTTCCCGCCCCCATCGAGAAAACGATT
TCTAAGACGAAAGGGCGCCCGAAAGCCCCACAGGTCTACACCATCCCGCC
CCCCAAAGAGCAGATGGCGAAAGACAAGGTGTCATTGACCTGCATGATTA
CCGACTTCTTTCCCGAGGATATCACCGTGGAATGGCAATGGAATGGCCAG
CCTGCTGAAAACTATAAGAACACCCAGCCCATCATGGATACTGATGGTTC
ATACTTTGTCTACAGTAAGCTTAATGTGCAGAAATCCAATTGGGAGGCTG
GGAACACTTTTACCTGTAGTGTGCTTCATGAAGGCCTGCACAACCACCAT
ACAGAAAAAAGTCTCTCACATAGCCCCGGA
(SEQ ID NO: 49)
MACNCQLMQDTPLLKFPCPRLILLFVLLIRLSQVSSDVDEQLSKSVKDKV
LLPCRYNSPHEDESEDRIYWQKHDKVVLSVIAGKLKVAPEYKNRTLYDNT
TYSLIILGLVLSDRGTYSCVVQKKERGTYEVKHLALVKLSIKADFSTPNI
TESGNPSADTKRITCFASGGFPKPRFSWLENGRELPGINTTISQDPESEL
YTISSQLDFNTTRNHTIKCLIKYGDAHVSEDFTWGSSSSGCKPCICTVPE
VSSVFIFPPKPKDVLTITLTPKVTCVVVDISKDDPEVQFSWFVDDVEVHT
AQTKPREEQFNSTFRSVSELPIMHQDWLNGKEFKCRVNSAAFPAPIEKTI
SKTKGRPKAPQVYTIPPPKEQMAKDKVSLTCMITDFFPEDITVEWQWNGQ
PAENYKNTQPIMDTDGSYFVYSKLNVQKSNWEAGNTFTCSVLHEGLHNHH
TEKSLSHSPG

Stop

TGA

connect to RE

RE sites (NotI and PacI)

| | |
|---|---|
| gcggccgcttaattaa | (SEQ ID NO: 26) |

connect to HCV 3′ TLS

Hepatitis C virus-3′ TLS (bases 8539-8637 of GenBank accession No. AJ242651)

(SEQ ID NO: 27)
ggtggctccatcttagccctagtcacggctagctgtgaaaggtccgtgag
ccgcttgactgcagagagtgctgatactggcctctctgcagatcaagt

connect to phi-8 segment M 3-prime end

Phi-8 segment M 3-prime Polymerase binding site (bases 4677-4741 of GenBank accession No. AF226852) with 3′ extension shown in upper case:

(SEQ ID NO: 37)
actgttgataaacaggacccggaagggtaacccgagagggggagtgaggc
ttcggcctccacttcGAAAATTTC

### REFERENCES

1. Sinclair, J. F., A. Tzagoloff, D. Levine, and L. Mindich. 1975. Proteins of bacteriophage phi6. J Virol 16:685-695.
2. McGraw, T., L. Mindich, and B. Frangione. 1986. Nucleotide sequence of the small double-stranded RNA segment of bacteriophage phi 6: novel mechanism of natural translational control. J Virol 58:142-151.
3. Gottlieb, P., S. Metzger, M. Romantschuk, J. Carton, J. Strassman, D. H. Bamford, N. Kalkkinen, and L. Mindich. 1988. Nucleotide sequence of the middle dsRNA segment of bacteriophage phi 6: placement of the genes of membrane-associated proteins. Virology 163:183-190.
4. Mindich, L., I. Nemhauser, P. Gottlieb, M. Romantschuk, J. Carton, S. Frucht, J. Strassman, D H. Bamford, and N. Kalkkinen. 1988. Nucleotide sequence of the large double-stranded RNA segment of bacteriophage phi 6: genes specifying the viral replicase and transcriptase. J Virol 62:1180-1185.
5. Mindich, L. 1999. Precise packaging of the three genomic segments of the double-stranded-RNA bacteriophage phi6. Microbiol. Mol. Biol. Rev. 63:149-160.
6. Mindich, L. 1988. Bacteriophage phi 6: a unique virus having a lipid-containing membrane and a genome composed of three dsRNA segments. Adv Virus Res 35:137-176.
7. Van Etten, J. L., A. K. Vidaver, R. K. Koski, and J. P. Burnett. 1974. Base composition and hybridization studies of the three double-stranded RNA segments of bacteriophage phi 6. J Virol 13:1254-1262.
8. Sands, J. A., and R. A. Lowlicht. 1976. Temporal origin of viral phospholipids of the enveloped bacteriophage phi 6. Can J Microbiol 22:154-158.
9. Bamford, D. H., and E. T. Palva. 1980. Structure of the lipid-containing bacteriophage phi 6. Disruption by Triton X-100 treatment. Biochim Biophys Acta 601:245-259.
10. Berger, H., and K. Kennedy. 1980. Physical measurements on the lipid-containing bacteriophage phi 6. Biochim Biophys Acta 633:68-76.
11. Olkkonen, V. M., and D. H. Bamford. 1987. The nucleocapsid of the lipid-containing double-stranded RNA bacteriophage phi 6 contains a protein skeleton consisting of a single polypeptide species. J Virol 61:2362-2367.
12. Johnson, M. D., 3rd, and L. Mindich. 1994. Plasmid-directed assembly of the lipid-containing membrane of bacteriophage phi 6. J Bacteriol 176:4124-4132.
13. Van Etten, J. L., A. K. Vidaver, R. K. Koski, and J. S. Semancik. 1973. RNA polymerase activity associated with bacteriophage phi 6. J Virol 12:464-471.
14. Huiskonen, J. T., F. de Haas, D. Bubeck, D. H. Bamford, S. D. Fuller, and S. J. Butcher. 2006. Structure of the bacteriophage phi6 nucleocapsid suggests a mechanism for sequential RNA packaging. Structure 14:1039-1048.
15. Pirttimaa, M. J., A. O. Paatero, M. J. Frilander, and D. H. Bamford. 2002. Nonspecific nucleoside triphosphatase P4 of double-stranded RNA bacteriophage phi6 is required for single-stranded RNA packaging and transcription. J Virol 76:10122-10127.
16. Poranen, M. M., R. Daugelavicius, P. M. Ojala, M. W. Hess, and D. H. Bamford. 1999. A novel virus-host cell membrane interaction. Membrane voltage-dependent endocytic-like entry of bacteriophage straight phi6 nucleocapsid. J Cell Biol 147:671-682.
17. Tuma, R., J. K. Bamford, D. H. Bamford, and G. J. Thomas, Jr. 1999. Assembly dynamics of the nucleocapsid shell subunit (P8) of bacteriophage phi6. Biochemistry 38:15025-15033.
18. Mindich, L., X. Qiao, J. Qiao, S. Onodera, M. Romantschuk, and D. Hoogstraten. 1999. Isolation of additional bacteriophages with genomes of segmented double-stranded RNA. J Bacteriol 181:4505-4508.
19. Hoogstraten, D., X. Qiao, Y. Sun, A. Hu, S. Onodera, and L. Mindich. 2000. Characterization of phi8, a bacteriophage containing three double-stranded RNA genomic segments and distantly related to Phi6. Virology 272:218-224.
20. Qiao, X., J. Qiao, S. Onodera, and L. Mindich. 2000. Characterization of phi 13, a bacteriophage related to phi 6 and containing three dsRNA genomic segments. Virology 275:218-224.
21. Onodera, S., V. M. Olkkonen, P. Gottlieb, J. Strassman, X. Y. Qiao, D. H. Bamford, and L. Mindich. 1992. Construction of a transducing virus from double-stranded RNA bacteriophage phi6: establishment of carrier states in host cells. J Virol 66:190-196.
22. Onodera, S., X. Qiao, J. Qiao, and L. Mindich. 1998. Directed changes in the number of double-stranded RNA genomic segments in bacteriophage phi6. Proc Natl Acad Sci USA 95:3920-3924.
23. Praszkier, J., and A. J. Pittard. 2005. Control of replication in I-complex plasmids. Plasmid 53:97-112.
24. Butcher, S. J., J. M. Grimes, E. V. Makeyev, D. H. Bamford, and D. I. Stuart. 2001. A mechanism for initiating RNA-dependent RNA polymerization. Nature 410:235-240.
25. Laurila, M. R., P. S. Salgado, D. I. Stuart, J. M. Grimes, and D. H. Bamford. 2005. Back-priming mode of phi6 RNA-dependent RNA polymerase. J Gen Virol 86:521-526.
26. Giron, J. A., J. G. Xu, C. R. Gonzalez, D. Hone, J. B. Kaper, and M. M. Levine. 1995. Simultaneous expression of CFA/I and CS3 colonization factor antigens of enterotoxigenic Escherichia coli by delta aroC, delta aroD Salmonella typhi vaccine strain CVD 908. Vaccine 13:939-946.
27. Hone, D. M., A. M. Harris, V. Lim, and M. M. Levine. 1994. Construction and characterization of isogenic O-antigen variants of Salmonella typhi. Mol. Microbiol. 13:525-530.
28. Hone, D., S. Attridge, L. van den Bosch, and J. Hackett. 1988. A chromosomal integration system for stabilization of heterologous genes in Salmonella-based vaccine strains. Microbial. Path. 5:407-418.

All references cited herein, are herein incorporated by reference in entirety.

An artisan will recognize that various changes and modifications can be made to the invention described herein, without departing from the spirit and the scope of the invention.

The instant application is a 371 of PCT Ser. No. US07/89209 which was filed 31 Dec. 2007, which claims benefit to US Ser. No. 60882679 filed 29 Dec. 2006 and to U.S. Ser. No. 60/972,097 filed 13 Sep. 2007, the contents of which are herein incorporated by reference in entirety.

1. A replication proficient double-stranded RNA (dsRNA) capsid (RPC).

2. The RPC of claim 1, further comprising an expressed transgene.

3. The RPC of claim 2, wherein said expressed transgene encodes a polypeptide.

4. The RPC of claim 3, wherein said polypeptide comprises an immunoregulatory activity.

5. The RPC of claim 3, wherein said polypeptide comprises an immunogen.

6. The RPC of claim 3, wherein said polypeptide specifically binds an antigen.

7. The RPC of claim 2, wherein said expressed transgene encodes a trait modifying RNA.

8. The RPC of claim 7, wherein said modifying RNA comprises an siRNA.

9. The RPC of claim 1, comprising a translation loop structure (TLS), a 3′ untranslated region (UTR) or both.

10. The RPC of claim 9, further comprising an expressed transgene.

11. The RPC of claim 1, further comprising an internal ribosome entry site (IRES), a cap independent translation enhancer (CITE) or both.

12. A composition comprising the RPC of claim 1.

13. The composition of claim 12, further comprising a cell.

14. A dsRNA capsid further comprising a TLS.

15. The capsid of claim 14, wherein an RNA comprising said TLS further comprises a 3′ UTR.

16. The capsid of claim 14, wherein said capsid is an rdsRN.

17. The capsid of claim 14, wherein said capsid is an rdsRP.

18. The capsid of claim 14, further comprising an expressed transgene.

19. The capsid of claim 18, wherein said expressed transgene encodes a polypeptide.

20. The capsid of claim 19, wherein said expressed transgene encodes a trait modifying RNA.

21. A composition comprising the capsid of claim 14.

22. The composition of claim 21, further comprising a cell.

23. The composition of claim 18, further comprising the product of said expressed transgene.


SEQUENCE LISTING (84 sequences)

SEQ ID NO: 1 - RNA, 10 nt, Pseudomonas phage phi-6
guuuuuuuac

SEQ ID NO: 2 - RNA, 10 nt, Artificial Sequence
  Description of Artificial Sequence Synthetic oligonucleotide
guuuuuuucc

SEQ ID NO: 3 - RNA, 10 nt, Pseudomonas phage phi-6
cucucucucu

SEQ ID NO: 4 - RNA, 10 nt, Artificial Sequence
  Description of Artificial Sequence Synthetic oligonucleotide
cucucuuccc

SEQ ID NO: 5 - DNA, 20 bp, Artificial Sequence
  Description of Artificial Sequence Synthetic oligonucleotide
ttctatagtgtcacctaaat

SEQ ID NO: 6 - RNA, 17 nt, Artificial Sequence
  Description of Artificial Sequence Synthetic oligonucleotide
ggauccugggaaacagg

SEQ ID NO: 7 - protein, 190 aa, West Nile Virus
SQIGLIEVLGKMPEHFMGKTWEALDTMYVVATAEKGGRAHRMALEELPDALQTIALIALLSVMTMGVFFLLMQRKGIGKIGLGGAVLGVATFFCWMAEVPGTKIAGMLLLSLLLMIVLIPEPEKQRSQTDNQLAVFLICVMTLVSAVAANEMGWLDKTKSDISSLFGQRIEVKENFSMGEFLLDLRPATA

SEQ ID NO: 8 - protein, 6 aa, Artificial Sequence
  Description of Artificial Sequence Synthetic peptide
SGGGGS

SEQ ID NO: 9 - protein, 8 aa, Artificial Sequence
  Description of Artificial Sequence Synthetic peptide
SPPPPPPS

SEQ ID NO: 10 - protein, 14 aa, Artificial Sequence
  Description of Artificial Sequence Synthetic peptide
SGGGGRTKRGGGGS

SEQ ID NO: 11 - RNA, 10 nt, Artificial Sequence
  Description of Artificial Sequence Synthetic oligonucleotide
ggaaaaaaag

SEQ ID NO: 12 - DNA, 30 bp, Artificial Sequence
  Description of Artificial Sequence Synthetic primer
gattacagatctatttaggtgacactatag

SEQ ID NO: 13 - DNA, 34 bp, Artificial Sequence
  Description of Artificial Sequence Synthetic primer
gaaattttcgaagtggaggccgaagcctcactcc

SEQ ID NO: 14 - DNA, 25 bp, Artificial Sequence
  Description of Artificial Sequence Synthetic primer
gaagtggaggccgaagcctcactcc

SEQ ID NO: 15 - DNA, 36 bp, Artificial Sequence
  Description of Artificial Sequence Synthetic primer
gaaattttcgaagcggaggaccgaagtcctcactcc

SEQ ID NO: 16 - DNA, 27 bp, Artificial Sequence
  Description of Artificial Sequence Synthetic primer
gaagcggaggaccgaagtcctcactcc

SEQ ID NO: 17 - DNA, 17 bp, Artificial Sequence
  Description of Artificial Sequence Synthetic oligonucleotide
atttaggtgacactata

SEQ ID NO: 18 - DNA, 187 bp, Pseudomonas phage phi-8
gaaattttcaaatcttttgactatttcgctggcatagctcttcggagtgaagccttccctgaaaggcgcgaaggtccccaccagctcggggtgattcgtgacatttcctgggatctcggagtcagctttgtctctaggagactgagcgttcggtctcaggtttaaactgagattgaggataaagaca

SEQ ID NO: 19 - DNA, 1101 bp, Pseudomonas phage phi-8
atgggtagaatctttcaactgttgatgcgcttaggcgttaaacagggtgcagcaagtgttggtaaagccgggatcgatgctggtagcaagcgattgctccagcagatcatgtccaaagacggtgctattcagctgtctaaggcactcggtttcaccgctgtggagcagatgtcgagtgaagtgctcgaagcgtatctctatgagatcgttgagcatcttctgctcgtcgacgaggccacgttggccgatgcgcttatggcgtgtatcaccgatgcaggtgatatcgccattgagcgtctgcttccttccgtagaggatgtcgacaaaggcgaggcgcttgccgccacgctgactgtcgtcttggctctcttctcgatgaacaaagaacaagctgaagagcttaaacgttcgatggcatcgaaaggcttgagtccggaccgggttaccctcggaggacagaccctgttgaccgtcaagtccactggtactggcctgacagagtatgacgctcaaggcaagaatggcgtccctcgcgggatgtctgctaacaagcgtactgcattgttcttcgtgctgtacacagtgatcagtacttcctggtccgtatacgatcactatggtgaggttaaagctggtctcgcacgaggcgagctacctcccagtgctgatcgtgttgaattgcgggcccccggttcctccgtaagtgcgatcgagcgtgagacacaacgcgcactgcaagaagaacagccgcgtgcattgccttcgggcagccgcaccgcggaacgggttgctgggccgacgcagggtgatgtccccgtgctcacacctccgccaggtcgattcaccttcaccggtgagggcgaccatcgtcccgatttcgcacaactcgctcgccagaacgacactgatggcgttgtgcggatcattgaactggatcgcattccagatgcaaggaaaatattagtcgatggtgaccatgactacttgctggacgccgctcaacagcgcgtcgctgccgatatcggggtatcgcccgagtcagtaggtcgattcgctgctctggtagccagtatcatcaacgcgaaggagaagcgttcgtga

SEQ ID NO: 20 - DNA, 12 bp, Artificial Sequence
  Description of Artificial Sequence Synthetic oligonucleotide
aggaggacagct

SEQ ID NO: 21 - DNA, 304 bp, Artificial Sequence
  Description of Artificial Sequence Synthetic polynucleotide
atgaccatgattacgccaagctcaatacgactcactatagggcccggtaccgagctcactagtttaattaaaagcttatcggccgaggtgagaagggtttcgatatcgagagaaacggttctcaccgcggccgcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcggccgc

SEQ ID NO: 22 - DNA, 337 bp, Hepatitis C Virus
atcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaa

SEQ ID NO: 23 - DNA, 14 bp, Artificial Sequence
  Description of Artificial Sequence Synthetic oligonucleotide
ggcgcgccgttaac

SEQ ID NO: 24 - DNA, 10 bp, Artificial Sequence
  Description of Artificial Sequence Synthetic oligonucleotide
cgccaccatg

SEQ ID NO: 25 - DNA, 684 bp, Artificial Sequence
  Description of Artificial Sequence Synthetic polynucleotide
gtgagcggcctgctgaaggagagtatgcgcatcaagatgtacatggagggcaccgtgaacggccactacttcaagtgcgagggcgagggcgacggcaaccccttcgccggcacccagagcatgagaatccacgtgaccgagggcgcccccctgcccttcgccttcgacatcctggccccctgctgcgagtacggcagcaggaccttcgtgcaccacaccgccgagatccccgacttcttcaagcagagcttccccgagggcttcacctgggagagaaccaccacctacgaggacggcggcatcctgaccgcccaccaggacaccagcctggagggcaactgcctgatctacaaggtgaaggtgcacggcaccaacttccccgccgacggccccgtgatgaagaacaagagcggcggctgggagcccagcaccgaggtggtgtaccccgagaacggcgtgctgtgcggccggaacgtgatggccctgaaggtgggcgaccggcacctgatctgccaccactacaccagctaccggagcaagaaggccgtgcgcgccctgaccatgcccggcttccacttcaccgacatccggctccagatgctgcggaagaagaaggacgagtacttcgagctgtacgaggccagcgtggcccggtacagcgacctgcccgagaaggccaactga

SEQ ID NO: 26 - DNA, 16 bp, Artificial Sequence
  Description of Artificial Sequence Synthetic oligonucleotide
gcggccgcttaattaa

SEQ ID NO: 27 - DNA, 98 bp, Hepatitis C Virus
ggtggctccatcttagccctagtcacggctagctgtgaaaggtccgtgagccgcttgactgcagagagtgctgatactggcctctctgcagatcaagt

SEQ ID NO: 28 - DNA, 121 bp, Pseudomonas phage phi-8
gcttagcggcaatcgaaccctccgataaggaggtttagcaaatccgcggctcttatgagctgtccgaaaggacaacccgaaagggggagtgaggacttcggtcctccgcttcgaaaatttc

SEQ ID NO: 29 - DNA, 262 bp, Pseudomonas phage phi-8
gaaattttcaaagtctttcggcaataagggtggaaatttcaaagagggtcgagccgacgaacctctgtagaaccgggaagtgcctgtctttacttgcgagagcaattgaactagggcagcaccgggggtcgataagcgcagaagtgaggcgcggggattgaagcaaatcacctaagcgtaaacgacggacctcgagggtggcggagtctacataggatcccctagctactagacagaaaccattcctaacaaggagatgcac

SEQ ID NO: 30 - DNA, 8 bp, Artificial Sequence
  Description of Artificial Sequence Synthetic oligonucleotide
ggccggcc

SEQ ID NO: 31 - DNA, 534 bp, Pseudomonas phage phi-8
atgcttaagatgctgctcgctacgcaaggtcttacgaccgcatccatcatggaaattctcactggttttgtggtgagacgggttcgactcgaggctcagcctttggtcgctagtatgatgccctccgttcgtgaggaggttgctcgagcgctcaacgacaaggcgtacaggcaagtactcgcaggggctggccaagttacactccgtgtattcaacggggttggtcagctggagacaccacttgtcgagattatcaaggatatcgcccgactgtcatcccctaccttcaagagcatgctcgagaaggcggagagaggcgagaatcttggtagtatgaccgacgaactcgcagagtccatagtggcagagttggttgcgctgatctcagccgatgcaaccgacgtgacgacagcactctctgtcccaggcgctgacgtggagcgctaccgtttgatcgttgattggctcaggggtcacatcaagtcgattgagcaaaaagatctgttcccggacatcatcgatttcctggagtag

SEQ ID NO: 32 - DNA, 8 bp, Artificial Sequence
  Description of Artificial Sequence Synthetic oligonucleotide
cctgcagg

SEQ ID NO: 33 - DNA, 861 bp, Artificial Sequence
  Description of Artificial Sequence Synthetic polynucleotide
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 34 - DNA, 816 bp, Artificial Sequence
  Description of Artificial Sequence Synthetic polynucleotide
atgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 35 - DNA, 15 bp, Artificial Sequence
  Description of Artificial Sequence Synthetic oligonucleotide
gccgccaccatgggc

SEQ ID NO: 36 - DNA, 1653 bp, Artificial Sequence
  Description of Artificial Sequence Synthetic polynucleotide
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 37 - DNA, 74 bp, Pseudomonas phage phi-8
actgttgataaacaggacccggaagggtaacccgagagggggagtgaggcttcggcctccacttcgaaaatttc

SEQ ID NO: 38 - DNA, 507 bp, Mus sp.
atgtactctatgcagctggcatcttgtgttactttgacactggtgcttctggtcaactcagccccaacttcatcttccaccagtagcagtaccgccgaggctcaacagcagcagcaacagcagcaacaacaacagcagcacctggagcaactgctcatggacctgcaagagttgctgtcaaggatggaaaactatagaaaccttaaactgccacggatgctgaccttcaagttctacctgcccaagcaagcaaccgaactcaaggatctgcaatgccttgaggatgaactgggccccctgcggcatgtgctggatctgacgcagtctaaatctttccagctcgaagacgcagaaaatttcattagcaacattcgggtgactgtggtaaaattgaaagggtctgacaacacttttgagtgccagttcgacgacgagtcagccaccgtcgtggactttttgagacgctggatcgccttttgccagtcaatcatatccactagtccccag

SEQ ID NO: 39 - protein, 169 aa, Mus sp.
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 40 - DNA, 147 bp, Rattus norvegicus
gggcccttcagccaaacacaacagcactctgcactgacctccatgctgagagctcagtttgccatgtaggccacacaagagccttgattgaagatgtgatgttctctctgaagagtatttaaagttttcaataaagtatataccctc

SEQ ID NO: 41 - DNA, 573 bp, West Nile Virus
atgggcggagtgctctgggatactccgagccctaaggagtacaaaaagggcgacactacaactggagtgtataggatcatgacaagaggattgctcgggagttatcaagcgggcgcaggggtgatggtcgagggggtgtttcatactctctggcacaccaccaaaggggctgccctcatgagcggtgaggggcggctggatccctactgggggagcgtcaaagaggacagattgtgctatggcgggccgtggaagctgcagcataagtggaacggccaagacgaagtgcagatgatcgtggtggagcctggcaaaaacgtgaaaaacgttcagaccaaaccaggcgtcttcaaaacaccagaaggggaaatcggcgctgtcacactggattttcctacgggcacctcaggttcccccatagtcgacaaaaatggagacgtcatcggcctgtatggaaacggggtcattatgccgaacggctcctatatctcagcgatcgtccagggggagcggatggacgagccaatcccggcagggttcgagccggaaatgcttcggaagaaacagataacc

SEQ ID NO: 42 - protein, 191 aa, West Nile Virus
MGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGQDEVQMIVVEPGKNVKNVQTKPGVFKTPEGEIGAVTLDFPTGTSGSPIVDKNGDVIGLYGNGVIMPNGSYISAIVQGERMDEPIPAGFEPEMLRKKQIT

SEQ ID NO: 43 - DNA, 18 bp, Artificial Sequence
  Description of Artificial Sequence Synthetic oligonucleotide
ggatcctcctcatccggg

SEQ ID NO: 44 - protein, 6 aa, Artificial Sequence
  Description of Artificial Sequence Synthetic peptide
GSSSSG

SEQ ID NO: 45 - DNA, 18 bp, Artificial Sequence
  Description of Artificial Sequence Synthetic oligonucleotide
caccaccaccatcaccac

SEQ ID NO: 46 - protein, 6 aa, Artificial Sequence
  Description of Artificial Sequence Synthetic 6xHis tag
HHHHHH

SEQ ID NO: 47 - DNA, 12 bp, Artificial Sequence
  Description of Artificial Sequence Synthetic oligonucleotide
gagaggacagct

SEQ ID NO: 48 - DNA, 1380 bp, Artificial Sequence
  Description of Artificial Sequence Synthetic polynucleotide
atggcctgtaattgtcaactgatgcaggatacccccttgcttaaatttccctgcccccgactgatcctcctctttgttctcctcatacgcctctctcaagtgtctagcgatgtagacgaacagctgtcaaaaagcgtgaaggataaagtcttgctgccctgtcgatacaacagcccgcacgaggacgaatccgaagatcgcatttactggcagaagcacgacaaggtggtgctctccgtgatcgctggcaagttgaaagtggctcccgagtataagaatcgaactctgtacgataataccacatactctttgattattttgggcctggttctgtctgatagggggacctatagctgcgttgtgcagaagaaagagcgcgggacatacgaagttaagcaccttgccctcgttaagctgtctataaaggctgacttcagcacaccgaatattactgaatctgggaaccctagcgcagatacaaaaaggatcacctgcttcgcttccggcgggttccctaaaccccggttctcatggcttgagaatggccgggagctgccaggaatcaacaccaccatttcacaggaccccgagtctgaactctacacaatcagcagccaactggattttaatacgacccgaaatcacacaattaagtgtctcattaaatatggagatgcccatgttagcgaagactttacttggggcagttcctcctcaggctgcaagccctgtatatgtacggtgccagaggtcagctctgtctttatattccctcctaagcctaaggacgttctgaccattaccctcactccgaaggtaacatgtgttgtggtggatatctcaaaggacgaccctgaggtacaattctcctggtttgtggatgacgtggaggttcacacggctcagacaaaaccacgggaagaacagtttaattcaacgtttcgctcagtgagtgagctccctatcatgcaccaggactggttgaatgggaaggagttcaaatgtcgcgttaacagcgcagcatttcccgcccccatcgagaaaacgatttctaagacgaaagggcgcccgaaagccccacaggtctacaccatcccgccccccaaagagcagatggcgaaagacaaggtgtcattgacctgcatgattaccgacttctttcccgaggatatcaccgtggaatggcaatggaatggccagcctgctgaaaactataagaacacccagcccatcatggatactgatggttcatactttgtctacagtaagcttaatgtgcagaaatccaattgggaggctgggaacacttttacctgtagtgtgcttcatgaaggcctgcacaaccaccatacagaaaaaagtctctcacatagccccgga

SEQ ID NO: 49 - protein, 460 aa, Artificial Sequence
  Description of Artificial Sequence Synthetic polypeptide
MACNCQLMQDTPLLKFPCPRLILLFVLLIRLSQVSSDVDEQLSKSVKDKVLLPCRYNSPHEDESEDRIYWQKHDKVVLSVIAGKLKVAPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWGSSSSGCKPCICTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 50 - RNA, 10 nt, Pseudomonas phage phi-6
guaaaaaaac

SEQ ID NO: 51 - RNA, 10 nt, Pseudomonas phage phi-6
agagagagag

SEQ ID NO: 52 - RNA, 10 nt, Pseudomonas phage phi-6
guuuuuuuac

SEQ ID NO: 53 - RNA, 10 nt, Pseudomonas phage phi-6
guuuuuuucc

SEQ ID NO: 54 - RNA, 10 nt, Pseudomonas phage phi-6
ggaaaaaaac

SEQ ID NO: 55 - RNA, 10 nt, Pseudomonas phage phi-6
gggaagagag

SEQ ID NO: 56 - RNA, 10 nt, Pseudomonas phage phi-8
gaaauuuauu

SEQ ID NO: 57 - RNA, 10 nt, Pseudomonas phage phi-8
uccucacuuc

SEQ ID NO: 58 - RNA, 10 nt, Pseudomonas phage phi-8
gaagugagga

SEQ ID NO: 59 - RNA, 10 nt, Pseudomonas phage phi-8
gaaguggagg

SEQ ID NO: 60 - RNA, 10 nt, Pseudomonas phage phi-8
aauaaauuuc

SEQ ID NO: 61 - RNA, 10 nt, Pseudomonas phage phi-8
gaaauuuuca

SEQ ID NO: 62 - RNA, 10 nt, Pseudomonas phage phi-8
gaaauuuucu

SEQ ID NO: 63 - RNA, 10 nt, Pseudomonas phage phi-8
ccuccacuuc

SEQ ID NO: 64 - RNA, 10 nt, Pseudomonas phage phi-8
ccuccgcuuc

SEQ ID NO: 65 - RNA, 10 nt, Pseudomonas phage phi-8
ugaaaauuuc

SEQ ID NO: 66 - RNA, 10 nt, Artificial Sequence
  Description of Artificial Sequence Synthetic oligonucleotide
agaaaauuuc

SEQ ID NO: 67 - RNA, 10 nt, Pseudomonas phage phi-13
gauaaaaacu

SEQ ID NO: 68 - RNA, 10 nt, Pseudomonas phage phi-13
gggucccccc

SEQ ID NO: 69 - RNA, 10 nt, Pseudomonas phage phi-13
ggggggaccc

SEQ ID NO: 70 - RNA, 10 nt, Pseudomonas phage phi-13
aguuuuuauc

SEQ ID NO: 71 - RNA, 10 nt, Pseudomonas phage phi-13
aguuuuuucc

SEQ ID NO: 72 - RNA, 10 nt, Pseudomonas phage phi-13
ggaaaaaacu

SEQ ID NO: 73 - RNA, 10 nt, Pseudomonas phage phi-13
cucucuucuc

SEQ ID NO: 74 - RNA, 10 nt, Pseudomonas phage phi-13
gagaagagag

SEQ ID NO: 75 - RNA, 10 nt, Pseudomonas phage phi-13
gggcuccucc

SEQ ID NO: 76 - RNA, 10 nt, Pseudomonas phage phi-13
ggaggagccc

SEQ ID NO: 77 - RNA, 10 nt, Artificial Sequence
  Description of Artificial Sequence Synthetic oligonucleotide
gccccgaccc

SEQ ID NO: 78 - RNA, 10 nt, Pseudomonas phage phi-13
gggucggggc

SEQ ID NO: 79 - DNA, 16 bp, Artificial Sequence
  Description of Artificial Sequence Synthetic oligonucleotide
ggccggcccctgcagg

SEQ ID NO: 80 - RNA, 10 nt, Artificial Sequence
  Description of Artificial Sequence Synthetic oligonucleotide
guuuuuuucc

SEQ ID NO: 81 - RNA, 10 nt, Pseudomonas phage phi-8
gaagcggagg

SEQ ID NO: 82 - RNA, 10 nt, Artificial Sequence
  Description of Artificial Sequence Synthetic oligonucleotide
ugaaaauuuc

SEQ ID NO: 83 - RNA, 10 nt, Pseudomonas phage phi-13
ggaaaaaaac

SEQ ID NO: 84 - RNA, 10 nt, Pseudomonas phage phi-13
guuuuuuucc